(19) United States
(12) Patent Application Publication
(10) Pub. No.: US 2010/0092992 A1
(43) Pub. Date: Apr. 15, 2010

(54) LYSINE ACETYLATION SITES

(76) Inventors: Peter Hornbeck, Magnolia, MA (US); Ting-Lei Gu, Woburn, MA (US); Ailan Guo, Burlington, MA (US); Charles Farnsworth, Concord, MA (US); Yu Li, Andover, MA (US); Jeffrey Mitchell, Nashua, NH (US)

Correspondence Address: Nancy Chiu Wilker, Ph.D.;Chief Intellectual Property Counsel, CELL SIGNALING TECHNOLOGY, INC., 3 Trask Lane, Danvers, MA, 01923, US

(21) Appl. No.: 12/310,292

(22) PCT Filed: Aug. 17, 2007
(86) PCT No.: PCT/US07/18367

§ 371 Date: Feb. 17, 2009

Related U.S. Application Data
(60) Provisional application No. 60/838,235, filed on Aug. 17, 2006.

(51) Int. Cl. G01N 33/53; C07K 16/00

(52) U.S. Cl. 435/7.1; 530/387.9

(57) ABSTRACT

The invention discloses 322 novel acetylation sites identified in various cancers, peptides (including AQUA peptides) comprising a acetylation site of the invention, antibodies specifically bind to a novel acetylation site of the invention, and diagnostic and therapeutic uses of the above.

### RELATED APPLICATIONS

Pursuant to 35 U.S.C. §119(e) this application claims the benefit of, and priority to, provisional application U.S. Ser. No. 60/838,235, filed Aug. 17, 2006, the disclosure of which is incorporated herein, in its entirety, by reference.

### FIELD OF THE INVENTION

The invention relates generally to novel lysine acetylation sites, methods and compositions for detecting, quantitating and modulating same.

### BACKGROUND OF THE INVENTION

The activation of proteins by post-translational modification is an important cellular mechanism for regulating most aspects of biological organization and control, including growth, development, homeostasis, and cellular communication. Protein acetylation, for example, plays a critical role in the etiology of many pathological conditions and diseases, including to mention but a few: cancer, developmental disorders, autoimmune diseases, and diabetes. Yet, in spite of the importance of protein modification, it is not yet well understood at the molecular level, due to the extraordinary complexity of signaling pathways, and the slow development of technology necessary to unravel it.

Protein acetylation plays a complex and critical role in the regulation of biological processes and may prove to be important to diagnostic or therapeutic targets for molecular medicine. Protein acetylation on lysine residues is a dynamic, reversible and highly regulated chemical modification. Historically, histones were perceived as the most important substrate of acetylation, if not the sole substrate. It was proposed 40 years ago that structural modification of histones by acetylation plays an important role in chromatin remodeling and gene expression. Two groups of enzymes, histone deacetylases (HDACs) and histone acetyltransferases (HATs), are responsible for deacetylating and acetylating the histones.

Recent studies have revealed that HDACs are involved in a much broader assay of biological processes. For example, HDAC6 has been implicated in the regulation of microtubules, growth factor-induced chemotaxis and misfolded protein stress response. See Cohen et al., Science, vol 245:42 (2004). Consistant with these non-histone functions, HDAC6 is mainly located to the cytoplasm.

A growing list of acetylated proteins is currently available. It shows that both cytoplasmic and nuclear proteins can undergo reversible acetylation, and protein acetylation can have the following effects on its function: 1) Protein stability. Both acetylation and ubiquitylation often occur on the same lysine, competition between these two modifications affects the protein stability. It has been shown that HDACs can decrease the half-life of some proteins by exposing the lysine for ubiquitylation. 2) Protein-protein interactions. It has been shown that acetylation induces STAT3 dimerization and subsequently nuclear translocation. In the case of nuclear DNA-damage-response protein Ku70, the deacetylated form of Ku70 sequesters BAX, the pro-apoptotic protein, in the cytoplasm and protects cells from apoptosis. In response to apoptotic stimuli, Ku70 becomes acetylated and subsequently releases Bax from its sequestration, leading to translocation of BAX to the mitochondria and activation of apoptotic cascade. 3) Protein translocation. As described for STAT3 and BAX, reversible acetylation affects the subcellular localization. In the case of STAT3, its nuclear localization signal contains lysine residues that favor nuclear retension when acetylated. 4) DNA binding. It have been shown that acetylation of p53 regulates its stability, its DNA binding and its transcriptional activity. Similarly, the DNA binding affinity of NF-kB and its transcriptional activation are also regulated by HATs and HDACs. See Minucci et al., Nature Cancer Reviews, 6: 38-51 (2005).

HATs and HDACs have been linked to pathogenesis of cancer. Specific HATs (p300 and CBP) are targets of viral oncoproteins (adenoviral E1A, human papilloma virus E6 and SV40 T antigen). See Eckner, R. et al., Cold Spring Harb. Symp. Quant. Biol., 59: 85-95 (1994). Structural alterations in HATs, including translocation, amplifications, deletions and point mutations have been found in various human cancers. See Iyer, N G. et al., Oncogene, 23: 4225-4231 (2004). For HDACs, increased expression of HDAC1 has been detected in gastric cancers, oesophageal squamous cell carcinoma, and prostate cancer. See Halkidou, K. et al., Prostate 59: 177-189 (2004). Increased expression of HDAC2 has been detected in colon cancer and has been shown to interact functionally with Wnt pathway. Knockdown of HDAC2 by siRNA in colon cancer cells resulted in cell death. See Zhu, P. et al., Cancer Cell, 5: 455-463 (2004). Increased expression of HDAC6 has been linked to better survival in breast cancer, See Zhang, Z. et al., Clin. Cancer Res., 10: 6962-6968 (2004), while reduced expression of HDAC5 and 10 have been associated with poor prognosis in lung cancer patients. See Osada, H. et al., Cancer, 112: 26-32 (2004).

HDAC inhibitors (HDACi) are promising new targeted anti-cancer agents, and first-generation HDACi in several clinical trials show significant activity against a spectrum of both hematologic and solid tumors at doses that are well tolerated by the patients. See Drummond, D C. et al., Annu. Rev. Pharmacol. Toxicol., 45: 495-528 (2005). However, the relationship between the toxicity of HDACi and their pharmacokinetic properties is still largely unknown, which makes it difficult to optimize HDACi treatment. More importantly the key targets for HDACi action are unknown. This makes it difficult to select patients who are most likely to respond to HDACi. Proposed surrogate markers, like measuring the level of acetylated histone from blood cells before and after treatment, should be serve as indicators of effectiveness, but these need to be validated clinically yet and do not always correlated with pharmacokinetic profile. Therefore, to identify the entire spectrum of acetylated proteins deserves a much more systematic experimental strategy which would optimally involve a dynamic map of the acetylated proteins and their functions.

Despite the identification of a few key molecules involved in protein acetylation signaling pathways, the vast majority of signaling protein changes underlying these pathways remains unknown. There is, therefore, relatively scarce information about acetylation-driven signaling pathways and acetylation sites relevant to the pathogenisis of cancer and other human diseases. This has hampered a complete and accurate understanding of how protein activation within signaling pathways may be driving different human diseases, including cancer.

Presently, diagnosis of carcinoma and other types of cancer is made by tissue biopsy and detection of different cell surface markers. However, misdiagnosis can occur since some carcinoma cases can be negative for certain markers and because these markers may not indicate which genes or protein kinases may be deregulated. Although the genetic translocations and/or mutations characteristic of a particular form of carcinoma can be sometimes detected, it is clear that other downstream effectors of constitutively active kinases having potential diagnostic, predictive, or therapeutic value, remain to be elucidated.

Accordingly, identification of downstream signaling molecules and acetylation sites involved in different types of diseases including for example, cancer and development of new reagents to detect and quantify these sites and proteins may lead to improved diagnostic/prognostic markers, as well as novel drug targets, for the detection and treatment of many diseases.

### SUMMARY OF THE INVENTION

The present invention provides in one aspect novel lysine acetylation sites (Table 1) identified in signal transduction proteins and pathways relevant to protein acetylation signaling. The novel sites occur in proteins such as: adaptor/scaffold proteins, apoptosis proteins, calcium-binding proteins, cell cycle regulation proteins, cell surface proteins, chormatin or DNA binding/repair/replication proteins, cytoskeletal proteins, enzyme proteins, g proteins or regulator proteins, proteases, phosphatases, receptor/channel/transporter/cell surface proteins, RNA binding proteins, transcriptional regulators, translational regulators, ubiquitan conjugating system, vesicle proteins and proteins of unknown function.

In another aspect, the invention provides peptides comprising the novel acetylation sites of the invention, and proteins and peptides that are mutated to eliminate the novel acetylation sites.

In another aspect, the invention provides modulators that modulate lysine acetylation at a novel acetylation site of the invention, including small molecules, peptides comprising a novel acetylation site, and binding molecules that specifically bind at a novel acetylation site, including but not limited to antibodies or antigen-binding fragments thereof.

In another aspect, the invention provides compositions for detecting, quantitating or modulating a novel acetylation site of the invention, including peptides comprising a novel acetylation site and antibodies or antigen-binding fragments thereof that specifically bind at a novel acetylation site. In certain embodiments, the compositions for detecting, quantitating or modulating a novel acetylation site of the invention are Heavy-Isotype Labeled Peptides (AQUA peptides) comprising a novel acetylation site.

In another aspect, the invention discloses acetylation site specific antibodies or antigen-binding fragments thereof. In one embodiment, the antibodies specifically bind to an amino acid sequence comprising a acetylation site identified in Table 1 when the lysine identified in Column D is acetylated, and do not significantly bind when the lysine is not acetylated. In another embodiment, the antibodies specifically bind to an amino acid sequence comprising an acetylation site when the lysine is not acetylated, and do not significantly bind when the lysine is acetylated.

In another aspect, the invention provides a method for making acetylation site-specific antibodies.

In another aspect, the invention provides compositions comprising a peptide, protein, or antibody of the invention, including pharmaceutical compositions.

In a further aspect, the invention provides methods of treating or preventing cancer in a subject, wherein the cancer is associated with the acetylation state of a novel acetylation site in Table 1, whether acetylated or deacetylated. In certain embodiments, the methods comprise administering to a subject a therapeutically effective amount of a peptide comprising a novel acetylation site of the invention. In certain embodiments, the methods comprise administering to a subject a therapeutically effective amount of an antibody or antigen-binding fragment thereof that specifically binds at a novel acetylation site of the invention.

In a further aspect, the invention provides methods for detecting and quantitating acetylation at a novel lysine acetylation site of the invention.

In another aspect, the invention provides a method for identifying an agent that modulates lysine acetylation at a novel acetylation site of the invention, comprising: contacting a peptide or protein comprising a novel acetylation site of the invention with a candidate agent, and determining the acetylation state or level at the novel acetylation site. A change in the acetylation state or level at the specified lysine in the presence of the test agent, as compared to a control; indicates that the candidate agent potentially modulates lysine acetylation at a novel acetylation site of the invention.

In another aspect, the invention discloses immunoassays for binding, purifying, quantifying and otherwise generally detecting the acetylation of a protein or peptide at a novel acetylation site of the invention.

Also provided are pharmaceutical compositions and kits comprising one or more antibodies or peptides of the invention and methods of using them.

### BRIEF DESCRIPTION OF THE DRAWINGS

FIG. 1 is a diagram depicting the immuno-affinity isolation and mass-spectrometric characterization methodology (IAP) used in the Examples to identify the novel acetylation sites disclosed herein.

FIG. 2 is a table (corresponding to Table 1) summarizing the 322 novel acetylation sites of the invention: Column A=the parent proteins from which the acetylation sites are derived; Column B=the SwissProt accession number for the human homologue of the identified parent proteins; Column C=the protein type/classification; Column D=the lysine residues at which acetylation occurs (each number refers to the amino acid residue position of the lysine in the parent human protein, according to the published sequence retrieved by the SwissProt accession number); Column E=flanking sequences of the phosphorylatable lysine residues; sequences (SEQ ID NOs) were identified using Trypsin digestion of the parent proteins; in each sequence, the lysine (see corresponding rows in Column D) appears in lowercase; Column F=the type of disease in which each of the acetylation site was discovered; Column G=the cell type(s)/Tissue/Patient Sample in which each of the acetylation site was discovered; and Column H=the SEQ ID NOs of the trypsin-digested peptides identified in Column E.

FIG. 3 is an exemplary mass spectrograph depicting the detection of the acetylation of lysine 179 in eEF1A-1, as further described in Example 1 (red and blue indicate ions detected in MS/MS spectrum); K* indicates the acetylated lysine (corresponds to lowercase “k” in Column E of Table 1; SEQ ID NO: 143).

FIG. 4 is an exemplary mass spectrograph depicting the detection of the acetylation of lysine 163 in SKP1A, as further described in Example 1 (red and blue indicate ions detected in MS/MS spectrum); K* indicates the acetylated lysine (corresponds to lowercase “k” in Column E of Table 1; SEQ ID NO: 163).

FIG. 5 is an exemplary mass spectrograph depicting the detection of the acetylation of lysine 52 in RPL11, as further described in Example 1 (red and blue indicate ions detected in MS/MS spectrum); K* indicates the acetylated lysine (corresponds to lowercase “k” in Column E of Table 1; SEQ ID NO: 157).

FIG. 6 is an exemplary mass spectrograph depicting the detection of the acetylation of lysine 25 in RPL13A, as further described in Example 1 (red and blue indicate ions detected in MS/MS spectrum); K* indicates the acetylated lysine (corresponds to lowercase “k” in Column E of Table 1; SEQ ID NO: 159).

FIG. 7 is an exemplary mass spectrograph depicting the detection of the acetylation of lysine 21 in RPLP2, as further described in Example 1 (red and blue indicate ions detected in MS/MS spectrum); K* indicates the acetylated lysine (corresponds to lowercase “k” in Column E of Table 1; SEQ ID NO: 166).

FIG. 8 is an exemplary mass spectrograph depicting the detection of the acetylation of lysine 482 in PPIL2, as further described in Example 1 (red and blue indicate ions detected in MS/MS spectrum); K* indicates the acetylated lysine (corresponds to lowercase “k” in Column E of Table 1; SEQ ID NO: 57).

### DETAILED DESCRIPTION OF THE INVENTION

The inventors have discovered and disclosed herein novel lysine acetylation sites in signaling proteins extracted from cancer cells, including carcinoma cells. The newly discovered acetylation sites significantly extend our knowledge of kinase substrates and of the proteins in which the novel sites occur. The disclosure herein of the novel acetylation sites and reagents including peptides and antibodies specific for the sites add important new tools for the elucidation of signaling pathways that are associate with a host of biological processes including cell division, growth, differentiation, develomental changes and disease. Their discovery in cancer cells (including carcinoma cells) provides and focuses further elucidation of the disease process. And, the novel sites provide additional diagnostic and therapeutic targets.

### 1. Novel Acetylation Sites in Cancer Cell Lines Including Carcinoma

In one aspect, the invention provides 322 novel lysine acetylation sites in signaling proteins from cellular extracts from a variety of human cancer-derived cell lines and tissue samples (such as H3255, sw480, etc., as further described below in Examples), identified using the techniques described in “Immunoaffinity Isolation of Modified Peptides From Complex Mixtures,” U.S. Patent Publication No. 20030044848, Rush et al., using Table 1 summarizes the identified novel acetylation sites.

These acetylation sites thus occur in proteins found in cancer. The sequences of the human homologues are publicly available in SwissProt database and their Accession numbers listed in Column B of Table 1. The novel sites occur in proteins such as: adaptor/scaffold proteins, apoptosis proteins, calcium-binding proteins, cell cycle regulation proteins, cell surface proteins, chormatin or DNA binding/repair/replication proteins, cytoskeletal proteins, enzyme proteins, g proteins or regulator proteins, proteases, phosphatases, receptor/channel/transporter/cell surface proteins, RNA binding proteins, transcriptional regulators, translational regulators, ubiquitan conjugating system, vesicle proteins and proteins of unknown function. (see Column C of Table 1).

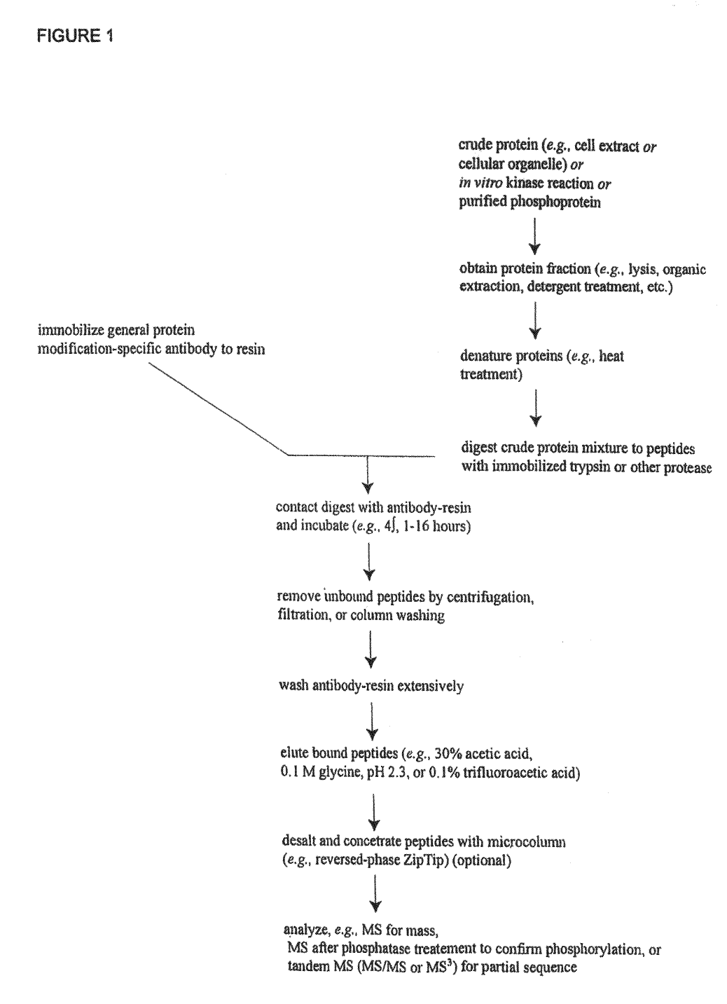

The novel acetylation sites of the invention were identified according to the methods described by Rush et al., U.S. Patent Publication No. 20030044848, which are herein incorporated by reference in its entirety. Briefly, acetylation sites were isolated and characterized by immunoaffinity isolation and mass-spectrometric characterization (IAP) (FIG. 1), using the following human carcinoma-derived cell lines and/or tissue samples: H23, H3255, H520, HCC78, HCC827, HCT116, HCT15, HCT8, HEP-G2, HeLa, Jurkat (pervanadate/calyculin), MV4;11, MV4;11 (SAHA), NB-4, NCI-H716, Nomo-1, OCI/AML2, OCI/AML3, RC-K8, RC-K8 (SAHA), SEM, SEM (SAHA), SIL-ALL, SNU-C2B, SW48, SW620, sw48 and sw480. In addition to the newly discovered acetylation sites (all having an acetylatable lysine), many known acetylation sites were also identified.

The immunoaffinity/mass spectrometric technique described in Rush et al, i.e., the “IAP” method, is described in detail in the Examples and briefly summarized below.

The IAP method generally comprises the following steps: (a) a proteinaceous preparation (e.g., a digested cell extract) comprising acetylpeptides from two or more different proteins is obtained from an organism; (b) the preparation is contacted with, at least one immobilized general acetylated-lysine-specific antibody; (c) at least one acetylpeptide specifically bound by the immobilized antibody in step (b) is isolated; and (d) the modified peptide isolated in step (c) is characterized by mass spectrometry (MS) and/or tandem mass spectrometry (MS-MS). Subsequently, (e) a search program (e.g., Sequest) may be utilized to substantially match the spectra obtained for the isolated, modified peptide during the characterization of step (d) with the spectra for a known peptide sequence. A quantification step, e.g., using SILAC or AQUA, may also be used to quantify isolated peptides in order to compare peptide levels in a sample to a baseline.

In the IAP method as disclosed herein, a general acetylated lysine-specific monoclonal antibody (commercially available from Cell Signaling Technology, Inc., Beverly, Mass., Cat #9681) may be used in the immunoaffinity step to isolate the widest possible number of acetyl-lysine containing peptides from the cell extracts.

As described in more detail in the Examples, lysates may be prepared from various cancer cell lines or tissue samples and digested with trypsin after treatment with DTT and iodoacetamide to alkylate cysteine residues. Before the immunoaffinity step, peptides may be pre-fractionated (e.g., by reversed-phase solid phase extraction using Sep-Pak C₁₈ columns) to separate peptides from other cellular components. The solid phase extraction cartridges may then be eluted (e.g., with acetonitrile). Each lyophilized peptide fraction can be redissolved and treated with acetyl-lysine specific antibody (e.g., CST Catalogue #8691) immobilized on protein Agarose. Immunoaffinity-purified peptides can be eluted and a portion of this fraction may be concentrated (e.g., with Stage or Zip tips) and analyzed by LC-MS/MS (e.g., using a ThermoFinnigan LCQ Deca XP Plus ion trap mass spectrometer or LTQ). MS/MS spectra can be evaluated using, e.g., the program Sequest with the NCBI human protein database.

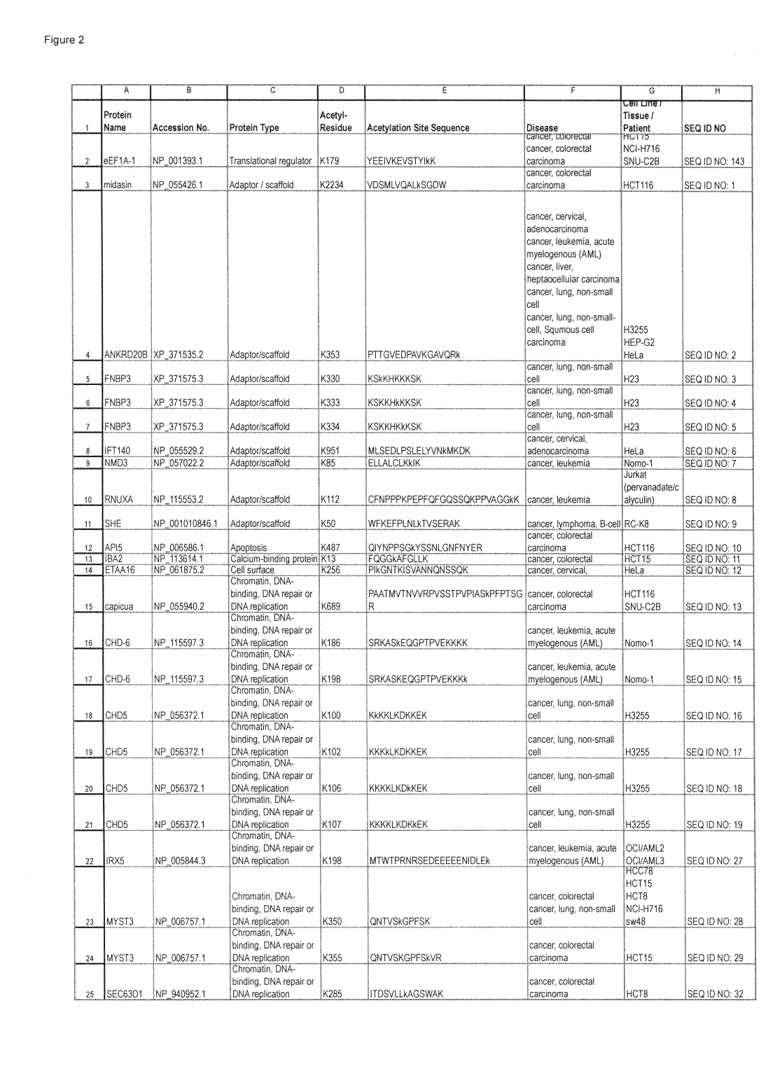

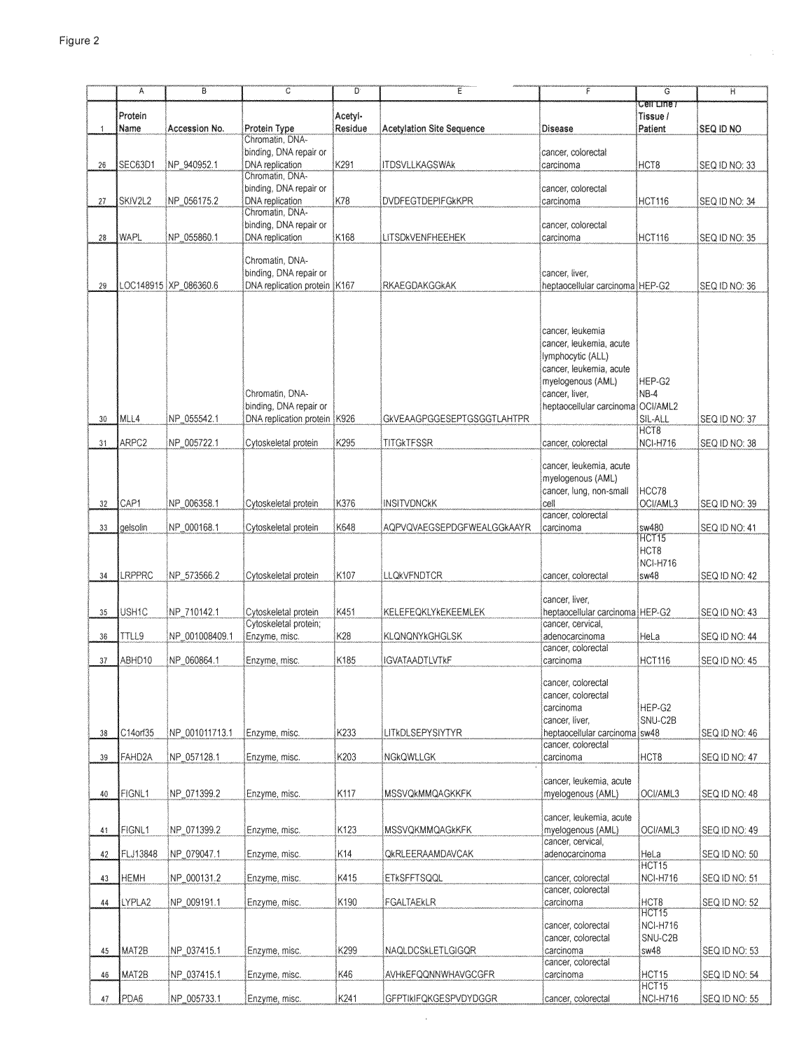

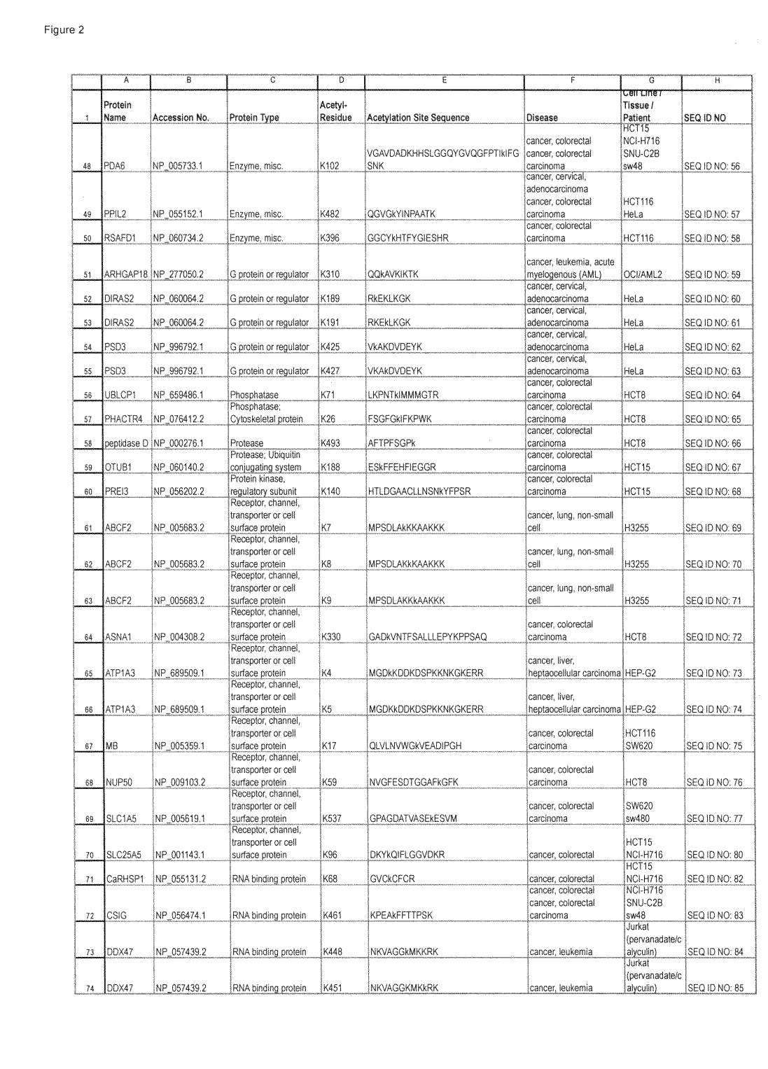

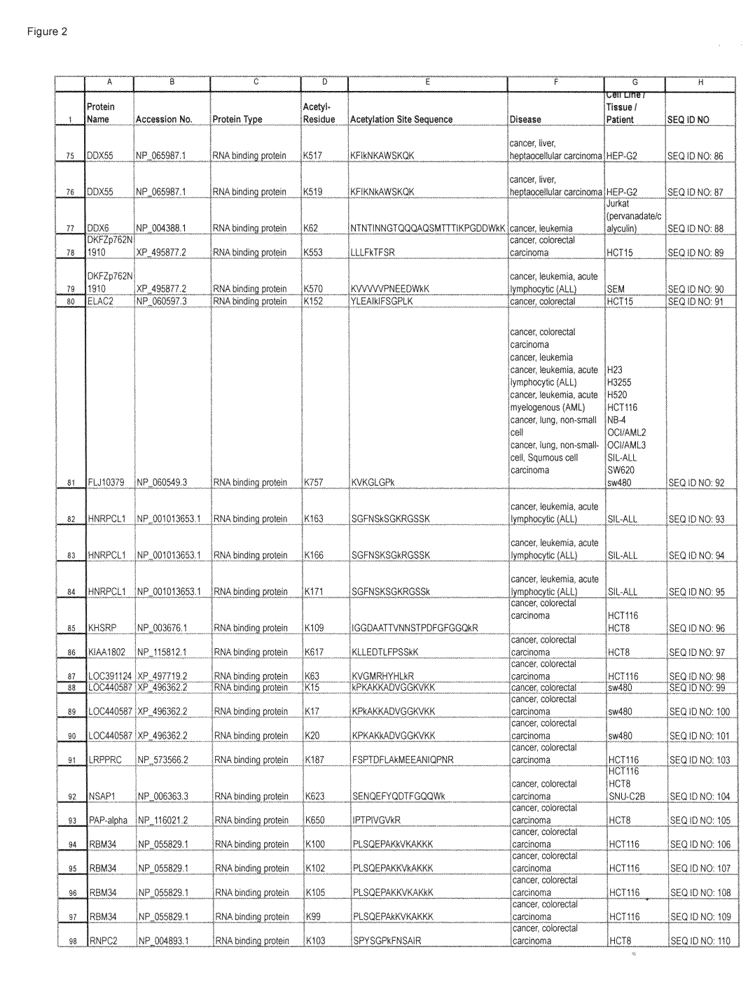

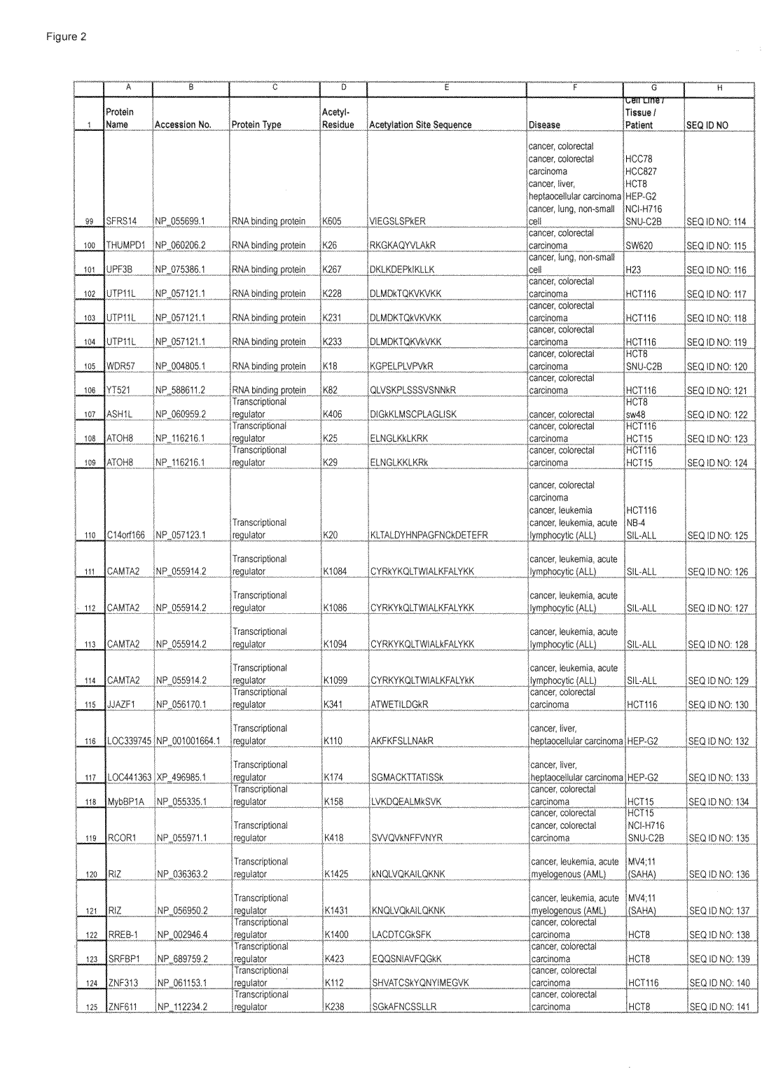

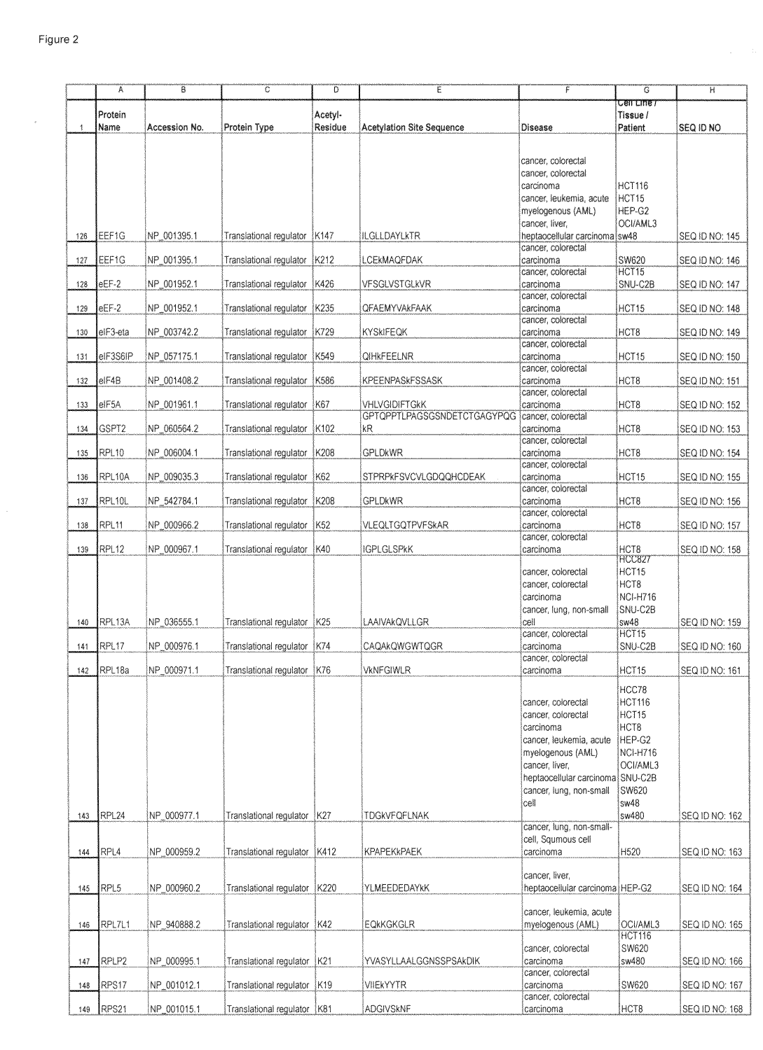

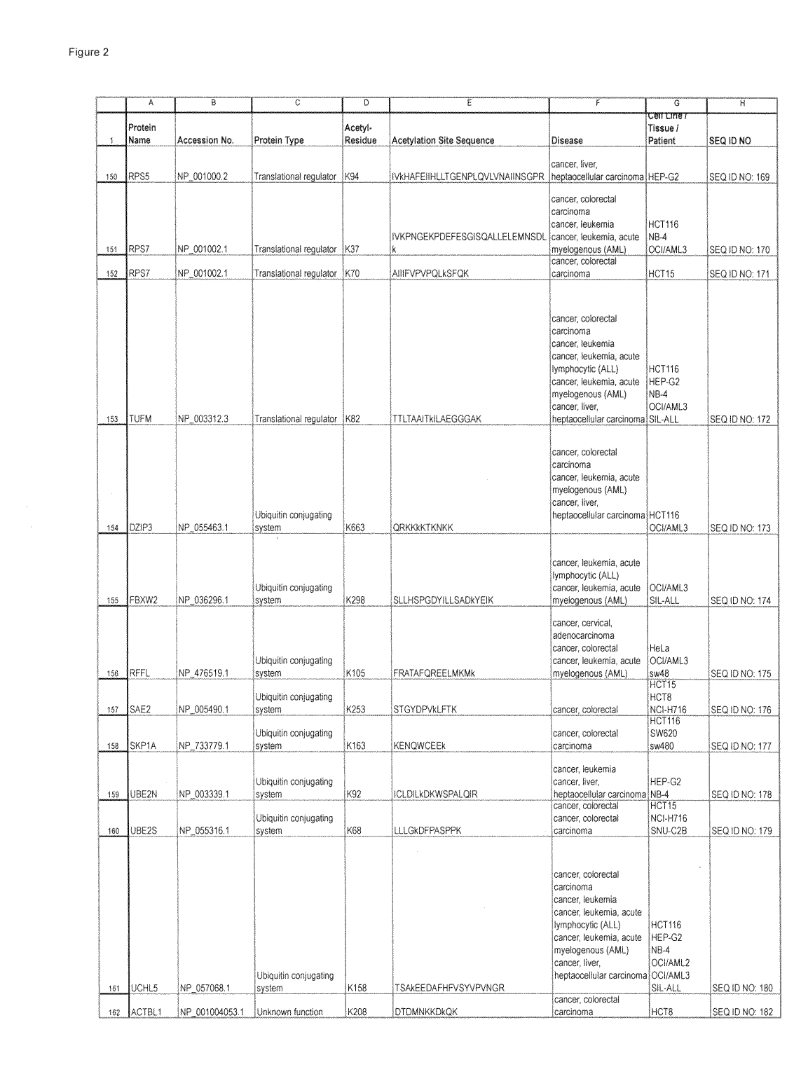

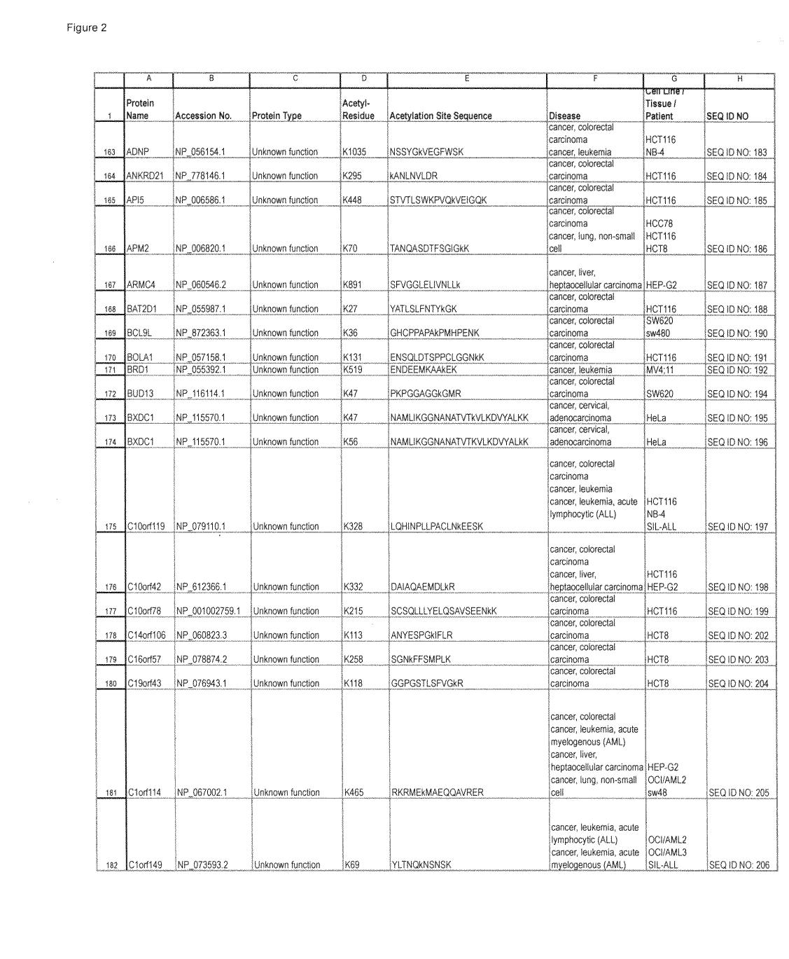

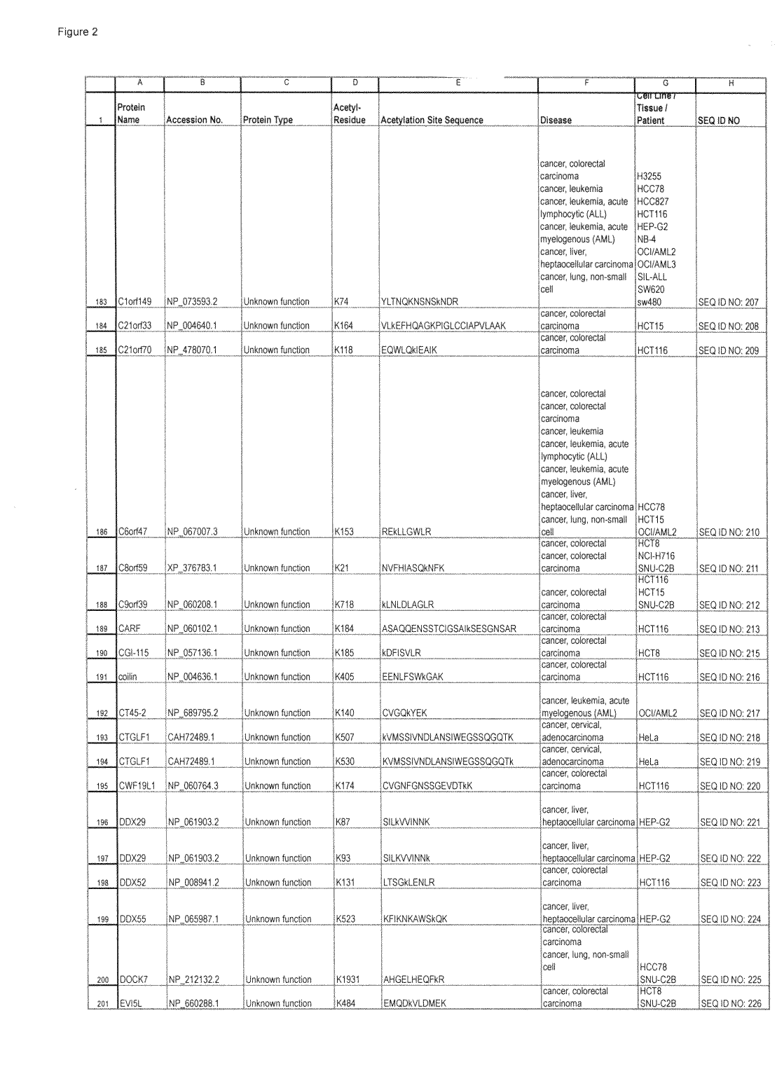

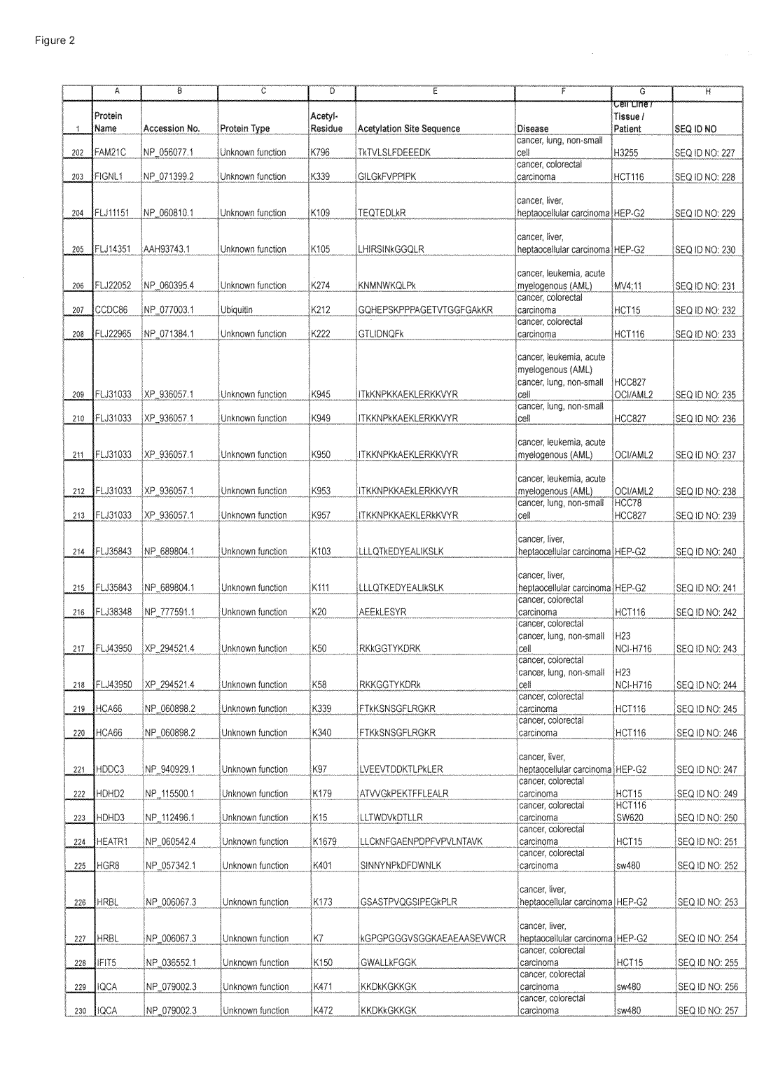

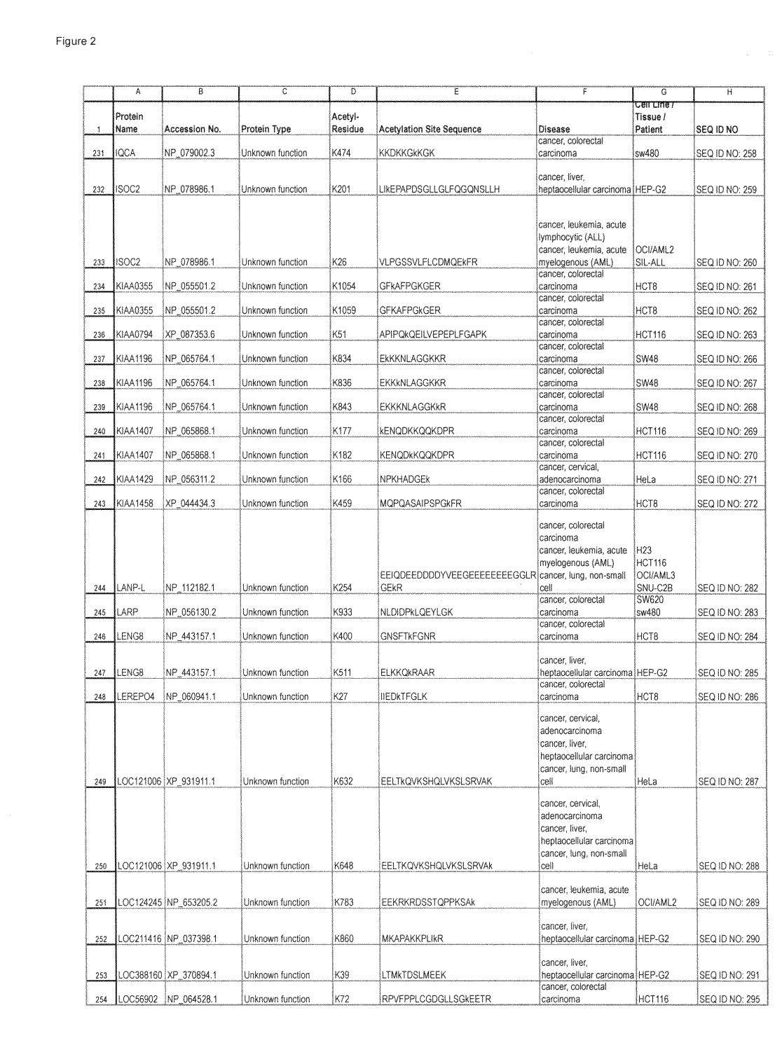

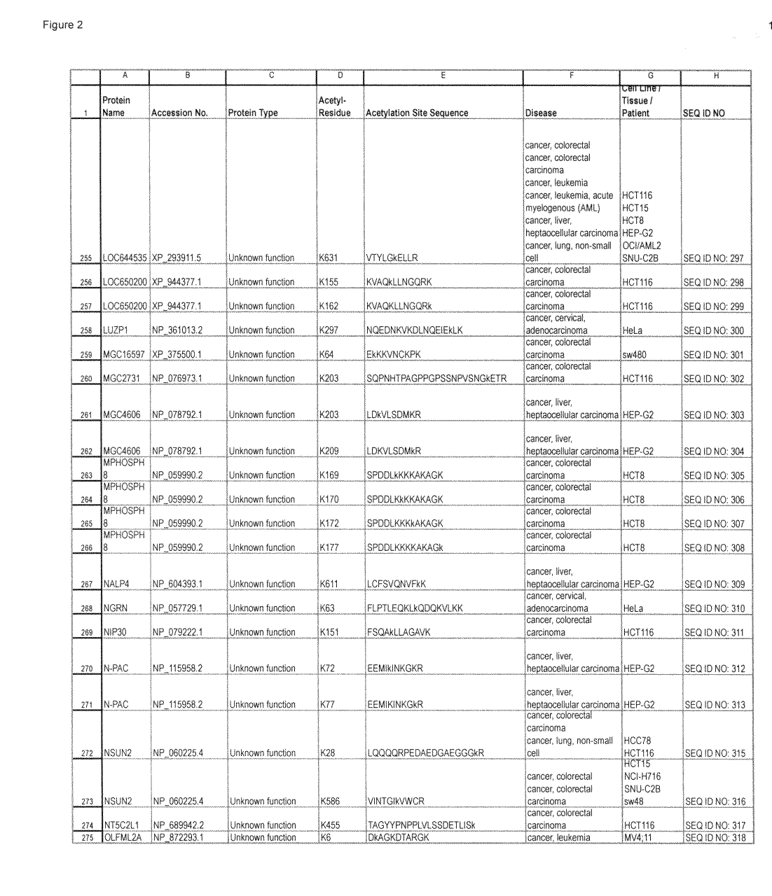

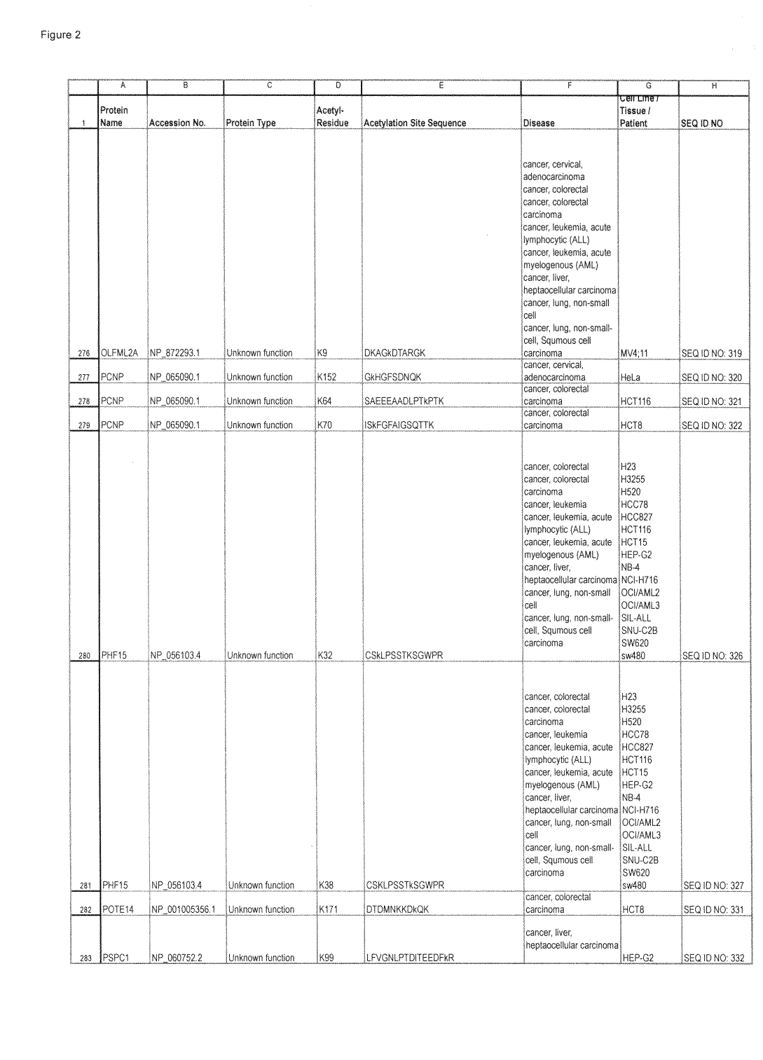

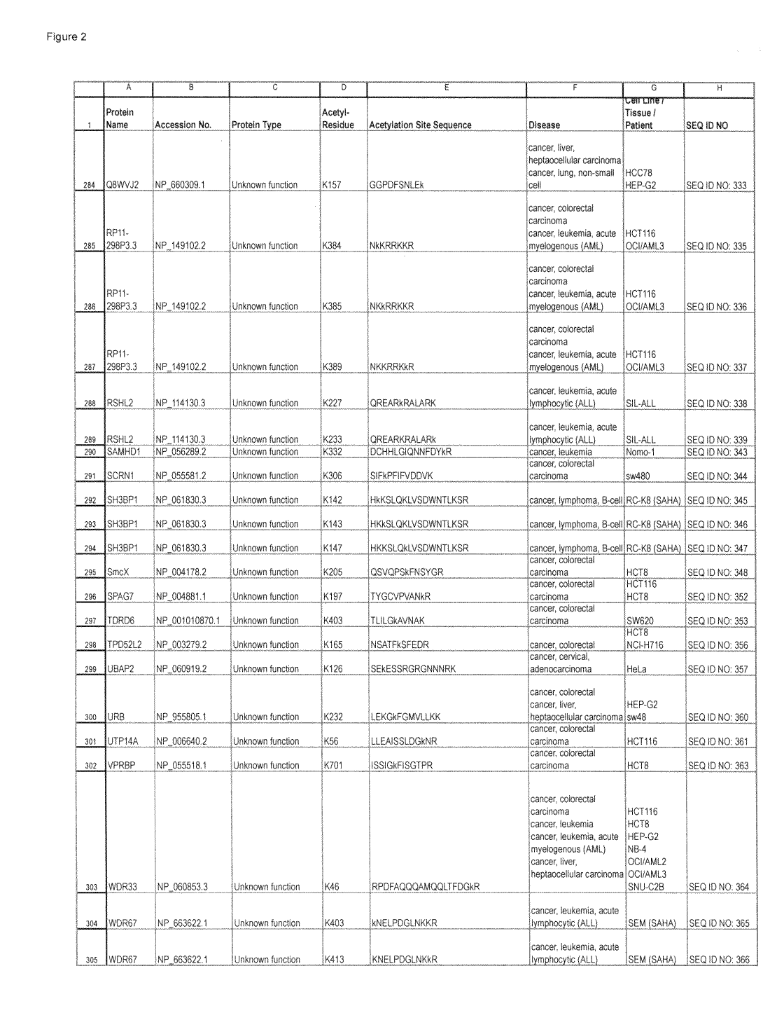

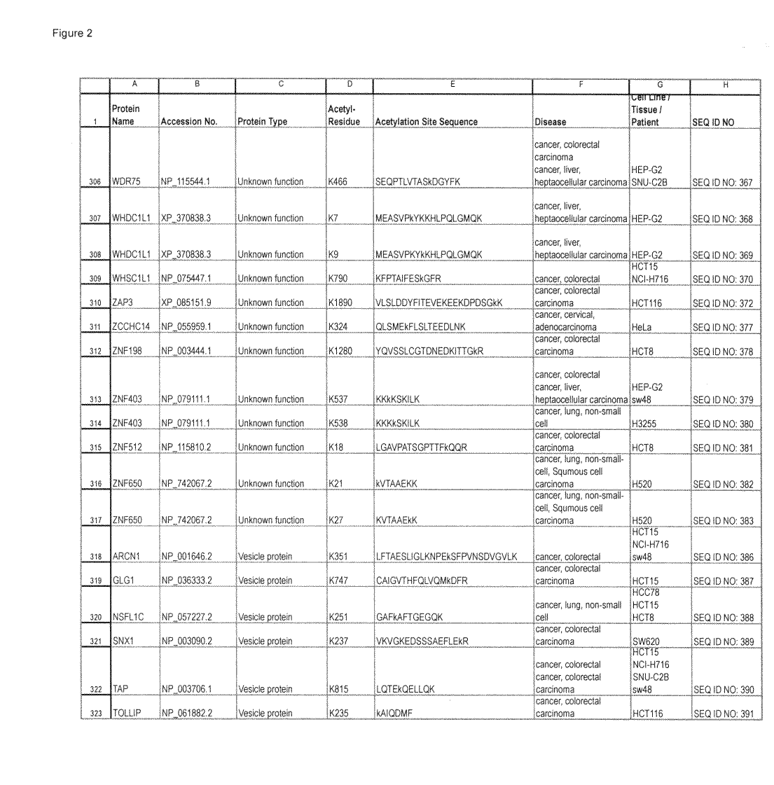

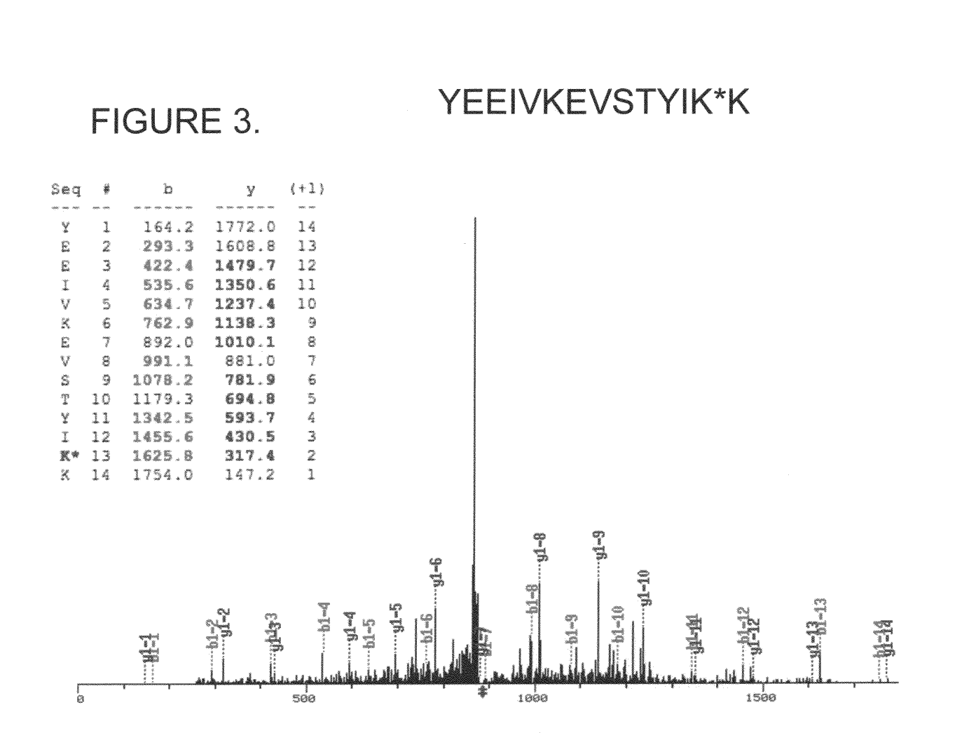

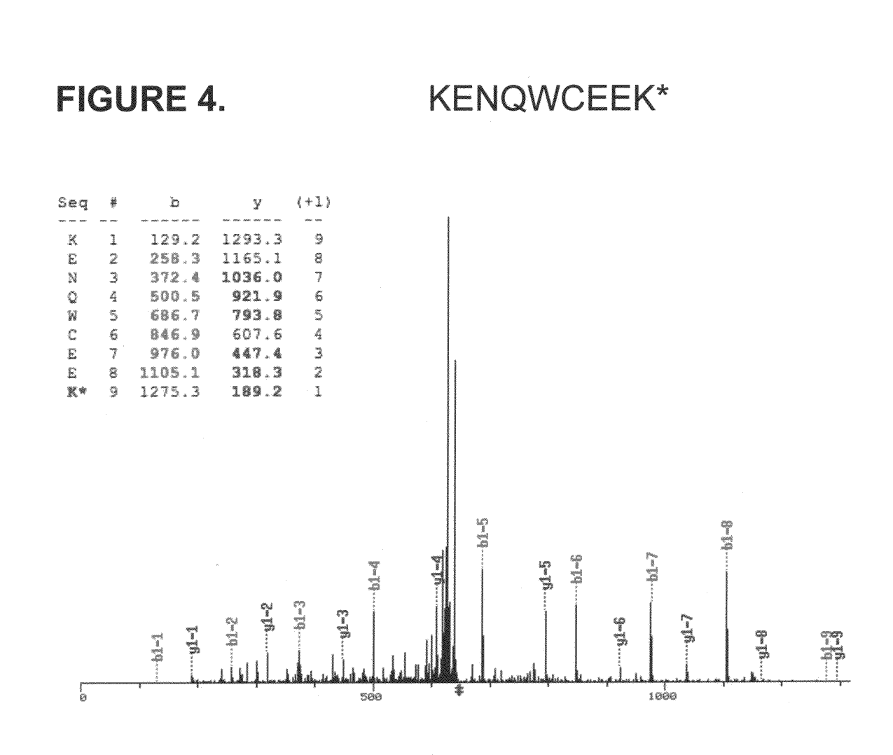

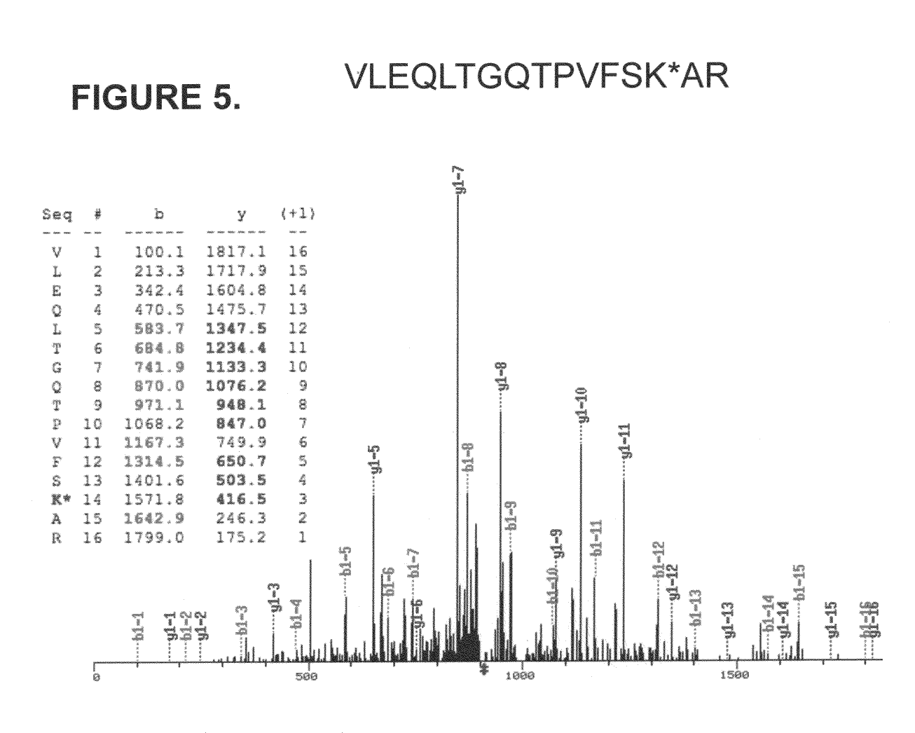

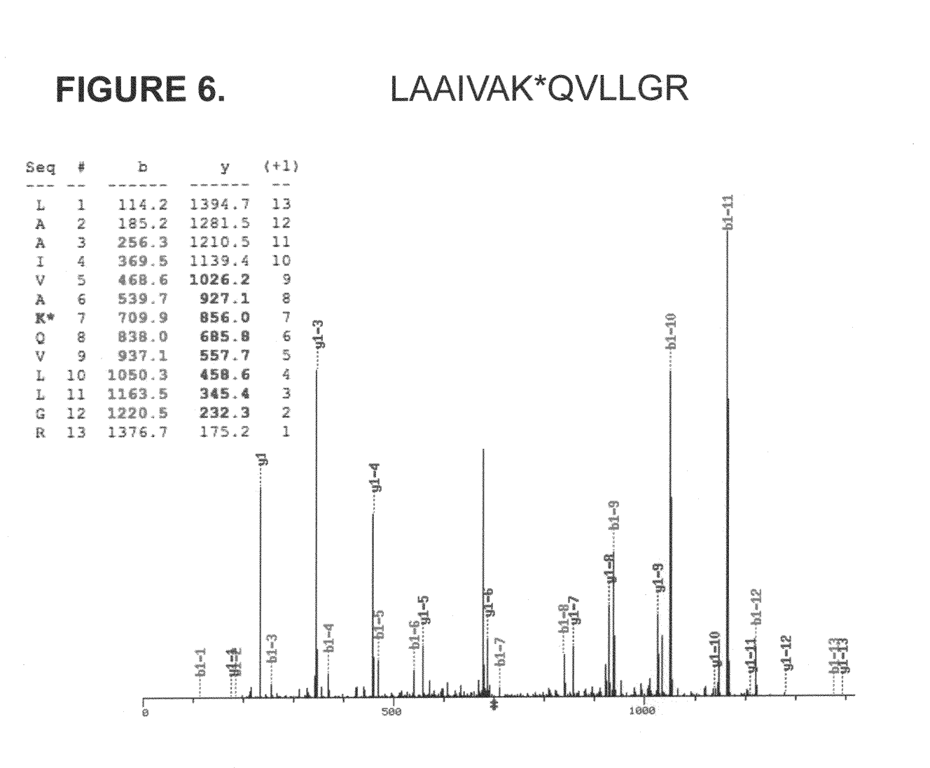

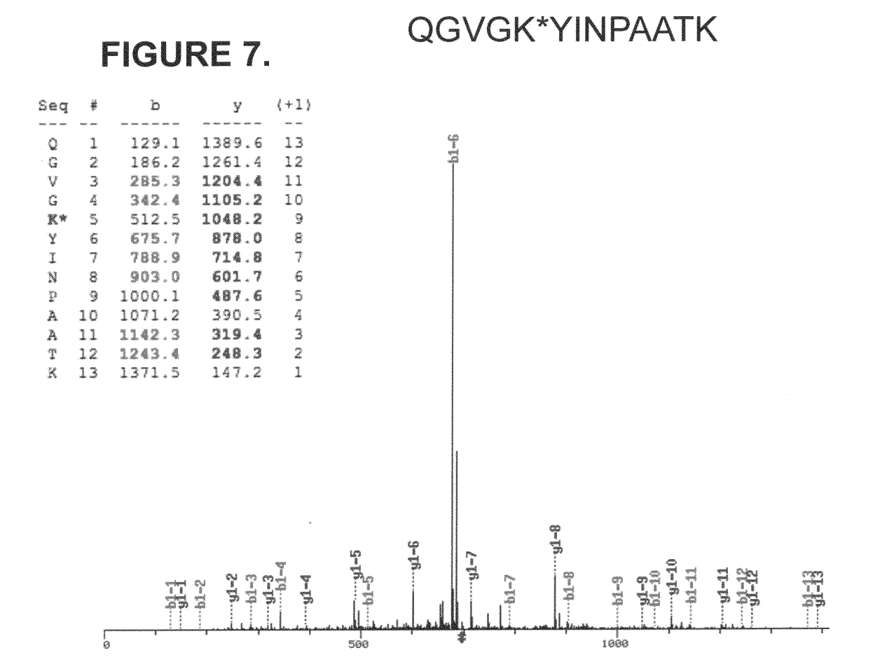

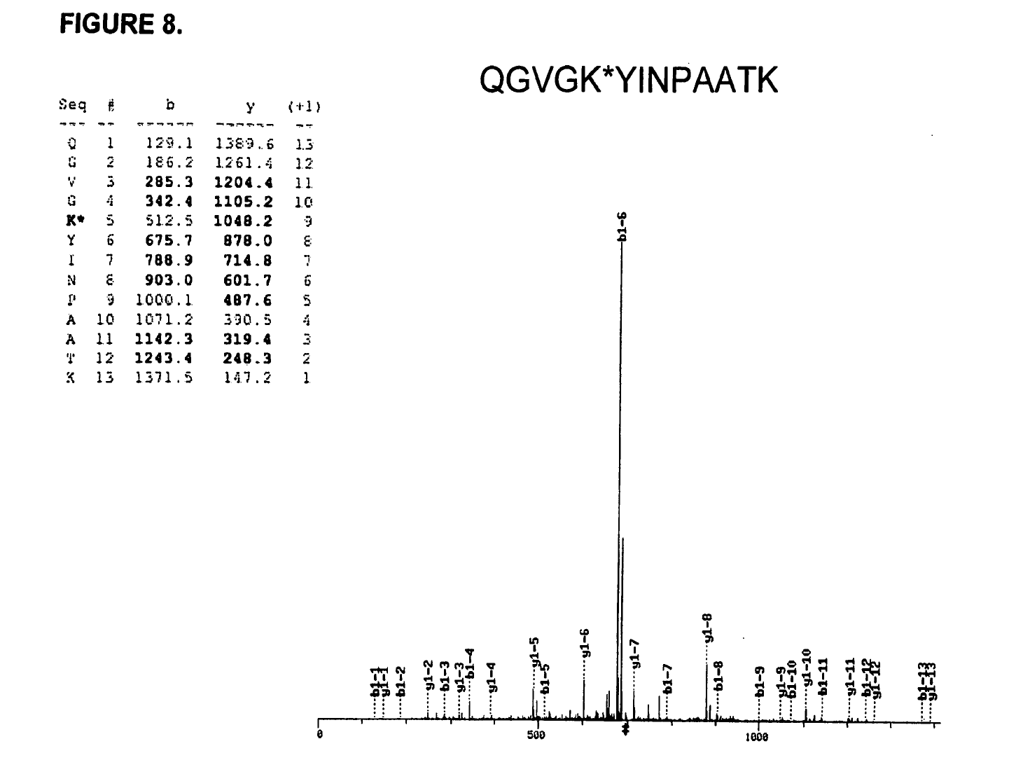

The novel acetylation sites identified are summarized in Table 1/FIG. 2. Column A lists the parent (signaling) protein in which the acetylation site occurs. Column D identifies the lysine residue at which acetylation occurs (each number refers to the amino acid residue position of the lysine in the parent human protein, according to the published sequence retrieved by the SwissProt accession number). Column E shows flanking sequences of the identified lysine residues (which are the sequences of trypsin-digested peptides). FIG. 2 also shows the particular type of cancer (see Column G) and cell line(s) (see Column F) in which a particular acetylation site was discovered.

TABLE 1
Novel Acetylation Sites in Cancer, Including Carcinoma.

| | A | | | D | | |
|---|---|---|---|---|---|---|
| | Protein | B | C | Phospho- | E | H |
| 1 | Name | Accession No. | Protein Type | Residue | Acetylation Site Sequence | SEQ ID NO |
| 2 | eEF1A-1 | NP_001393.1 | Translational | K179 | YEEIVKEVSTYIkK | SEQ ID NO: 143 |
| | | | regulator | | | |
| 3 | midasin | NP_055426.1 | Adaptor/scaffold | K2234 | VDSMLVQALkSGDW | SEQ ID NO: 1 |
| 4 | ANKRD20B | XP_371535.2 | Adaptor/scaffold | K353 | PTTGVEDPAVKGAVQRk | SEQ ID NO: 2 |
| 5 | FNBP3 | XP_371575.3 | Adaptor/scaffold | K330 | KSkKHKKKSK | SEQ ID NO: 3 |
| 6 | FNBP3 | XP_371575.3 | Adaptor/scaffold | K333 | KSKKHkKKSK | SEQ ID NO: 4 |
| 7 | FNBP3 | XP_371575.3 | Adaptor/scaffold | K334 | KSKKHKkKSK | SEQ ID NO: 5 |
| 8 | IFT140 | NP_055529.2 | Adaptor/scaffold | K951 | MLSEDLPSLELYVNkMKDK | SEQ ID NO: 6 |
| 9 | NMD3 | NP_057022.2 | Adaptor/scaffold | K85 | ELLALCLKkIK | SEQ ID NO: 7 |
| 10 | RNUXA | NP_115553.2 | Adaptor/scaffold | K112 | CFNPPPKPEPFQFGQSSQKPPVAGGkK | SEQ ID NO: 8 |
| 11 | SHE | NP_001010846.1 | Adaptor/scaffold | K50 | WFKEFPLNLkTVSERAK | SEQ ID NO: 9 |
| 12 | API5 | NP_006586.1 | Apoptosis | K487 | QIYNPPSGkYSSNLGNFNYER | SEQ ID NO: 10 |
| 13 | IBA2 | NP_113614.1 | Calcium-binding | K13 | FQGGkAFGLLK | SEQ ID NO: 11 |
| | | | protein | | | |
| 14 | ETAA16 | NP_061875.2 | Cell surface | K256 | RIkGNTKISVANNQNSSQK | SEQ ID NO: 12 |
| 15 | capicua | NP_055940.2 | Chromatin, DNA- | K689 | PAATMVTNVVRPVSSTPVPIASkPFPTSGR | SEQ ID NO: 13 |
| | | | binding, DNA | | | |
| | | | repair or DNA | | | |
| | | | replication | | | |
| 16 | CHD-6 | NP_115597.3 | Chromatin, DNA- | K186 | SRKASkEQGPTPVEKKKK | SEQ ID NO: 14 |
| | | | binding, DNA | | | |
| | | | repair or DNA | | | |
| | | | replication | | | |
| 17 | CHD-6 | NP_115597.3 | Chromatin, DNA- | K198 | SRKASKEQGPTPVEKKKk | SEQ ID NO: 15 |
| | | | binding, DNA | | | |
| | | | repair or DNA | | | |
| | | | replication | | | |
| 18 | CHD5 | NP_057372.1 | Chromatin, DNA- | K100 | KkKKLKDKKEK | SEQ ID NO: 16 |
| | | | binding, DNA | | | |
| | | | repair or DNA | | | |
| | | | replication | | | |
| 19 | CHD5 | NP_056372.1 | Chromatin, DNA- | K102 | KKKkLKDKKEK | SEQ ID NO: 17 |
| | | | binding, DNA | | | |
| | | | repair or DNA | | | |
| | | | replication | | | |
| 20 | CHD5 | NP_056372.1 | Chromatin, DNA- | K106 | KKKKLKDkKEK | SEQ ID NO: 18 |
| | | | binding, DNA | | | |
| | | | repair or DNA | | | |
| | | | replication | | | |
| 21 | CHD5 | NP_056372.1 | Chromatin, DNA- | K107 | KKKKLKDKkEK | SEQ ID NO: 19 |
| | | | binding, DNA | | | |
| | | | repair or DNA | | | |
| | | | replication | | | |
| 22 | IRX5 | NP_005844.3 | Chromatin, DNA- | K198 | MTWTPRNRSEDEEEEENIDLEk | SEQ ID NO: 27 |
| | | | binding, DNA | | | |
| | | | repair or DNA | | | |
| | | | replication | | | |
| 23 | MYST3 | NP_006757.1 | Chromatin, DNA- | K350 | QNTVSkGPFSK | SEQ ID NO: 28 |
| | | | binding, DNA | | | |
| | | | repair or DNA | | | |
| | | | replication | | | |
| 24 | MYST3 | NP_006757.1 | Chromatin, DNA- | K355 | QNTVSKGPFSkVR | SEQ ID NO: 29 |
| | | | binding, DNA | | | |
| | | | repair or DNA | | | |
| | | | replication | | | |
| 25 | SEC63D1 | NP_040952.1 | Chromatin, DNA- | K285 | ITDSVLLkAGSWAK | SEQ ID NO: 32 |
| | | | binding, DNA | | | |
| | | | repair or DNA | | | |
| | | | replication | | | |
| 26 | SEC63D1 | NP_040952.1 | Chromatin, DNA- | K291 | ITDSVLLKAGSWAk | SEQ ID NO: 33 |
| | | | binding, DNA | | | |
| | | | repair or DNA | | | |
| | | | replication | | | |
| 27 | SKIV2L2 | NP_056175.2 | Chromatin, DNA- | K78 | DVDFEGTDEPIFGkKPR | SEQ ID NO: 34 |
| | | | binding, DNA | | | |
| | | | repair or DNA | | | |
| | | | replication | | | |
| 28 | WAPL | NP_055860.1 | Chromatin, DNA- | K168 | LITSDkVENFHEEHEK | SEQ ID NO: 35 |
| | | | binding, DNA | | | |
| | | | repair or DNA | | | |
| | | | replication | | | |
| 29 | LDC148915 | XP_086360.6 | Chromatin, DNA- | K167 | RKAEGDAKGGkAK | SEQ ID NO: 36 |
| | | | binding, DNA | | | |
| | | | repair or DNA | | | |
| | | | replication | | | |
| | | | protein | | | |
| 30 | MLL4 | NP_055542.1 | Chromatin, DNA- | K926 | GkVEAAGPGGESEPTGSGGTLAHTPR | SEQ ID NO: 37 |
| | | | binding, DNA | | | |
| | | | repair or DNA | | | |
| | | | replication | | | |
| | | | protein | | | |
| 31 | ARPC2 | NP_005722.1 | Cytoskeletal | K295 | TITGkTFSSR | SEQ ID NO: 38 |
| | | | protein | | | |
| 32 | CAP1 | NP_006358.1 | Cytoskeletal | K376 | INSITVDNCkK | SEQ ID NO: 39 |
| | | | protein | | | |
| 33 | getsolin | NP_000168.1 | Cytoskeletal | K648 | AQPVQVAEGSEPDGFWEALGGkAAYR | SEQ ID NO: 41 |
| | | | protein | | | |
| 34 | LRPPRC | NP_573566.2 | Cytoskeletal | K107 | LLQkVFNDTCR | SEQ ID NO: 42 |
| | | | protein | | | |
| 35 | USH1C | NP_710142.1 | Cytoskeletal | K451 | KELEFEQKLYkEKEEMLEK | SEQ ID NO: 43 |
| | | | protein | | | |
| 36 | TTLL9 | NP_001008409.1 | Cytoskeletal | K28 | KLQNQNYkGHGLSK | SEQ ID NO: 44 |
| | | | protein; Enzyme | | | |
| | | | misc. | | | |
| 37 | ABHD10 | NP_060864.1 | Enzyme, misc. | K185 | IGVATAADTLVTkF | SEQ ID NO: 45 |
| 38 | C14orf35 | NP_001011713.1 | Enzyme, misc. | K233 | LITkDLSEPYSIYTYR | SEQ ID NO: 46 |
| 39 | FAHD2A | NP_057128.1 | Enzyme, misc. | K203 | NGkQWLLGK | SEQ ID NO: 47 |
| 40 | FIGNL1 | NP_071399.2 | Enzyme, misc. | K117 | MSSVQkMMQAGKKFK | SEQ ID NO: 48 |
| 41 | FIGNL1 | NP_071399.2 | Enzyme, misc. | K123 | MSSVQKMMQAGkKFK | SEQ ID NO: 49 |
| 42 | FLJ13848 | NP_079047.1 | Enzyme, misc. | K14 | QkRLEERAAMDAVCAK | SEQ ID NO: 50 |
| 43 | HEMH | NP_000131.2 | Enzyme, misc. | K415 | ETkSFFTSQQL | SEQ ID NO: 51 |
| 44 | LYPLA2 | NP_009191.1 | Enzyme, misc. | K190 | FGALTAEkLR | SEQ ID NO: 52 |
| 45 | MAT2B | NP_037415.1 | Enzyme, misc. | K299 | MAQLDCSkLETLGIGQR | SEQ ID NO: 53 |
| 46 | MAT2B | NP_037415.1 | Enzyme, misc. | K46 | AVHkEFQQNNWHAVGCGFR | SEQ ID NO: 54 |
| 47 | PDA6 | NP_005733.1 | Enzyme, misc. | K241 | GFPTIkIFQKGESPVDYDGGR | SEQ ID NO: 55 |
| 48 | PDA6 | NP_005733.1 | Enzyme, misc. | K102 | VGAVDADKHHSLGGQYGVQGFPTIkIFGSNK | SEQ ID NO: 56 |
| 49 | PPIL2 | NP_055152.1 | Enzyme, misc. | K482 | QGVGkYINPAATK | SEQ ID NO: 57 |
| 50 | RSAFD1 | NP_060734.2 | Enzyme, misc. | K396 | GGCYkHTFYGIESHR | SEQ ID NO: 58 |
| 51 | ARHGAP18 | NP_277052.2 | G protein or | K310 | QQkAVKIKTK | SEQ ID NO: 59 |
| | | regulator | | | | |
| 52 | DIRAS2 | NP_060064.2 | G protein or | K189 | RkEKLKGK | SEQ ID NO: 60 |
| | | regulator | | | | |
| 53 | DIRAS2 | NP_060064.2 | G protein or | K191 | RKEkLKGK | SEQ ID NO: 61 |
| | | regulator | | | | |
| 54 | PSD3 | NP_996792.1 | G protein or | K425 | VkAKDVDEYK | SEQ ID NO: 62 |
| | | regulator | | | | |
| 55 | PSD3 | NP_996792.1 | G protein or | K427 | KLPNTkIMMMGTR | SEQ ID NO: 63 |
| | | regulator | | | | |
| 56 | UBLCP1 | NP_659486.1 | Phosphatase | K71 | KLPNTkIMMMGTR | SEQ ID NO: 64 |
| 57 | PHACTR4 | NP_076412.2 | Phosphatase; | K26 | FSGFGkIFKPWK | SEQ ID NO: 65 |
| | | | Cytoskeletal | | | |
| | | | protein | | | |
| 58 | peptidase D | NP_000276.1 | Protease | K493 | AFTPFSGPk | SEQ ID NO: 66 |
| 59 | OTUB1 | NP_060140.2 | Protease, | K188 | ESkFFEHFIEGGR | SEQ ID NO: 67 |
| | | | Ubiquitin | | | |
| | | | conjugating | | | |
| | | | system | | | |
| 60 | PREI3 | NP_056202.2 | Protein kinase; | K140 | HTLDGAACLLNSNkYFPSR | SEQ ID NO: 68 |
| | | | regulatory subunit | | | |
| 61 | ABCF2 | NP_005683.2 | Receptor, | K7 | MPSDLAkKKAAKKK | SEQ ID NO: 69 |
| | | | channel, | | | |
| | | | transporter or cell | | | |
| | | | surface protein | | | |
| 62 | ABCF2 | NP_005683.2 | Receptor, | K8 | MPSDLAKkKAAKKK | SEQ ID NO: 70 |
| | | | channel, | | | |
| | | | transporter or cell | | | |
| | | | surface protein | | | |
| 63 | ABCF2 | NP_005683.2 | Receptor, | K9 | MPSDLAKKkAAKKK | SEQ ID NO: 71 |
| | | | channel, | | | |
| | | | transporter or cell | | | |
| | | | surface protein | | | |
| 64 | ASNA1 | NP_005408.2 | Receptor, | K330 | GADkVNTFSALLLEPYKPPSAQ | SEQ ID NO: 72 |
| | | | channel, | | | |
| | | | transporter or cell | | | |
| | | | surface protein | | | |
| 65 | ATP1A3 | NP_689509.1 | Receptor, | K4 | MGDkKDDKDSPKKNKGKERR | SEQ ID NO: 73 |
| | | | channel, | | | |
| | | | transporter or cell | | | |
| | | | surface protein | | | |
| 66 | ATP1A3 | NP_689509.1 | Receptor, | K5 | MGDKkDDKDSPKKNKGKERR | SEQ ID NO: 74 |
| | | | channel, | | | |
| | | | transporter or cell | | | |
| | | | surface protein | | | |
| 67 | MB | NP_005359.1 | Receptor, | K17 | QLVLNVWGkVEADIPGH | SEQ ID NO: 75 |
| | | | channel, | | | |
| | | | transporter or cell | | | |
| | | | surface protein | | | |
| 68 | NUP50 | NP_009103.2 | Receptor, | K59 | MVGFESDTGGAFkGFK | SEQ ID NO: 76 |
| | | | channel, | | | |
| | | | transporter or cell | | | |
| | | | surface protein | | | |
| 69 | SLC1A5 | NP_005619.1 | Receptor, | K537 | GPAGDATVASEkESVM | SEQ ID NO: 77 |
| | | | channel, | | | |
| | | | transporter or cell | | | |
| | | | surface protein | | | |
| 70 | SLC25A5 | NP_001143.1 | Receptor, | K96 | DKYkQIFLGGVDKR | SEQ ID NO: 80 |
| | | | channel, | | | |
| | | | transporter or cell | | | |
| | | | surface protein | | | |
| 71 | CaRHSP1 | NP_055131.2 | RNA binding | K68 | GVCkCFCR | SEQ ID NO: 82 |
| | | | protein | | | |
| 72 | CSIG | NP_056474.1 | RNA binding | K461 | KPEAkFFTTPSK | SEQ ID NO: 83 |
| | | | protein | | | |
| 73 | DDX47 | NP_057439.2 | RNA binding | K448 | NKVAGGkMKKRK | SEQ ID NO: 84 |
| | | | protein | | | |
| 74 | DDX47 | NP_057439.2 | RNA binding | K451 | NKVAGGKMKkRK | SEQ ID NO: 85 |
| | | | protein | | | |
| 75 | DDX55 | NP_065987.1 | RNA binding | K517 | KFIkNKAWSKQK | SEQ ID NO: 86 |
| | | | protein | | | |
| 76 | DDX55 | NP_065987.1 | RNA binding | K519 | KFIKNkAWSKQK | SEQ ID NO: 87 |
| | | | protein | | | |
| 77 | DDX6 | NP_004388.1 | RNA binding | K62 | NTNTINNGTQQQAQSMTTTIKPGKKWkK | SEQ ID NO: 88 |
| | | | protein | | | |
| 78 | DKFZp762 | XP_495877.2 | RNA binding | K553 | LLLFkTFSR | SEQ ID NO: 89 |
| | N1910 | | protein | | | |
| 79 | DKFZp762 | XP_495877.2 | RNA binding | K570 | KVVVVVPNEEDWkK | SEQ ID NO: 90 |
| | N1910 | | protein | | | |
| 80 | ELAC2 | NP_060597.3 | RNA binding | K152 | YLEAIkIFSGPLK | SEQ ID NO: 91 |
| | | | protein | | | |
| 81 | FLJ10379 | NP_060549.3 | RNA binding | K757 | KVKGLGPk | SEQ ID NO: 92 |
| | | | protein | | | |
| 82 | HNRPCL1 | NP_001013653.1 | RNA binding | K163 | SGFNSkSGKRGSSK | SEQ ID NO: 93 |
| | | | protein | | | |
| 83 | HNRPCL1 | NP_001013653.1 | RNA binding | K166 | SGFNSKSGkRGSSK | SEQ ID NO: 94 |
| | | | protein | | | |
| 84 | HNRPCL1 | NP_001013653.1 | RNA binding | K171 | SGFNSKSGKRGSSk | SEQ ID NO: 95 |
| | | | protein | | | |
| 85 | KHSRP | NP_003676.1 | RNA binding | K109 | IGGDAATTVNNSTPDFGFGGQkR | SEQ ID NO: 96 |
| | | | protein | | | |
| 86 | KIAA1802 | NP_115612.1 | RNA binding | K617 | KLLEDTLFPSSkK | SEQ ID NO: 97 |
| | | | protein | | | |
| 87 | LOC391124 | XP_497719.2 | RNA binding | K63 | KVGMRHYHLkR | SEQ ID NO: 98 |
| | | | protein | | | |
| 88 | LOC440587 | XP_496362.2 | RNA binding | K15 | kPKAKKADVGGKVKK | SEQ ID NO: 99 |
| | | | protein | | | |
| 89 | LOC440587 | XP_496362.2 | RNA binding | K17 | KPkAKKADVGGKVKK | SEQ ID NO: 100 |
| | | | protein | | | |
| 90 | LOC440587 | XP_496362.2 | RNA binding | K20 | KPKAKkADVGGKVKK | SEQ ID NO: 101 |
| | | | protein | | | |
| 91 | LRPPRC | NP_573566.2 | RNA binding | K187 | FSPTDFLAkMEEANIQPNR | SEQ ID NO: 103 |
| | | | protein | | | |
| 92 | NSAP1 | NP_006363.3 | RNA binding | K623 | SENQEFYQDTFGQQWk | SEQ ID NO: 104 |
| | | | protein | | | |
| 93 | PAP-alpha | NP_116021.2 | RNA binding | K650 | IPTPIVGVkR | SEQ ID NO: 105 |
| | | | protein | | | |
| 94 | RBM34 | NP_055829.1 | RNA binding | K100 | PLSQEPAKkVKAKKK | SEQ ID NO: 106 |
| | | | protein | | | |
| 95 | RBM34 | NP_055829.1 | RNA binding | K102 | PLSQEPAKKVkAKKK | SEQ ID NO: 107 |
| | | | protein | | | |
| 96 | RMB34 | NP_055829.1 | RNA binding | K105 | PLSQEPAKKVKAKkK | SEQ ID NO: 108 |
| | | | protein | | | |
| 97 | RBM34 | NP_055829.1 | RNA binding | K99 | PLSQEPAKkKVKAKKK | SEQ ID NO: 109 |
| | | | protein | | | |
| 98 | RNPC2 | NP_004839.1 | RNA binding | K103 | SPYSGPkFNSAIR | SEQ ID NO: 110 |
| | | | protein | | | |
| 99 | SFRS14 | NP_055699.1 | RNA binding | K605 | VIEGSLSPkER | SEQ ID NO: 114 |
| | | | protein | | | |
| 100 | THUMPD1 | NP_060206.2 | RNA binding | K26 | RKGKAQYVLAkR | SEQ ID NO: 115 |
| | | | protein | | | |
| 101 | UPF3B | NP_075386.1 | RNA binding | K267 | DKLKDEPkIKLLK | SEQ ID NO: 116 |
| | | | protein | | | |
| 102 | UTP11L | NP_057121.1 | RNA binding | K228 | DLMDkTQKVKVKK | SEQ ID NO: 117 |
| | | | protein | | | |
| 103 | UTP11L | NP_057121.1 | RNA binding | K231 | DLMDKTQkVKVKK | SEQ ID NO: 118 |
| | | | protein | | | |
| 104 | UTP11L | NP_057121.1 | RNA binding | K233 | DLMDKTQKVkVKK | SEQ ID NO: 119 |
| | | | protein | | | |
| 105 | WDR57 | NP_004805.1 | RNA binding | K18 | KGPELPLVPVkR | SEQ ID NO: 120 |
| | | | protein | | | |
| 106 | YT521 | NP_588611.2 | RNA binding | K62 | QLVSKPLSSSVSNNkR | SEQ ID NO: 121 |
| | | | protein | | | |
| 107 | ASH1L | NP_060959.2 | Transcriptional | K406 | DIGkKLMSCPLAGLISK | SEQ ID NO: 122 |
| | | | regulator | | | |
| 108 | ATOH8 | NP_116126.1 | Transcriptional | K25 | ELNGLKkLKRK | SEQ ID NO: 123 |
| | | | regulator | | | |
| 109 | ATOH8 | NP_116216.1 | Transcriptional | K29 | ELNGLKKLKRk | SEQ ID NO: 124 |
| | | | regulator | | | |
| 110 | C14orf166 | NP_057123.1 | Transcriptional | K20 | KLTALDYHNPAGFNCkDETEFR | SEQ ID NO: 125 |
| | | | regulator | | | |
| 111 | CAMTA2 | NP_055914.2 | Transcriptional | K1084 | CYRkYKQLTWIALKFALYKK | SEQ ID NO: 126 |
| | | | regulator | | | |
| 112 | CAMTA2 | NP_055914.2 | Transcriptional | K1086 | CYRKYkQLTWIALKFALYKK | SEQ ID NO: 127 |
| | | | regulator | | | |
| 113 | CAMTA2 | NP_055914.2 | Transcriptional | K1094 | CYRKYKQLTWIALkFALYKK | SEQ ID NO: 128 |
| | | | regulator | | | |
| 114 | CAMTA2 | NP_055914.2 | Transcriptional | K1099 | CYRKYKQLTWIALKFALYkK | SEQ ID NO: 129 |
| | | | regulator | | | |
| 115 | JJAZF1 | NP_056170.1 | Transcriptional | K341 | ATWETILDGkR | SEQ ID NO: 130 |
| | | | regulator | | | |
| 116 | LOC339745 | NP_001001664.1 | Transcriptional | K110 | AKFKFSLLNAkR | SEQ ID NO: 132 |
| | | | regulator | | | |
| 117 | LOC441363 | XP_496985.1 | Transcriptional | K174 | SGMACKTTATISSk | SEQ ID NO: 133 |
| | | | regulator | | | |
| 118 | MbyBP1A | NP_055335.1 | Transcriptional | K158 | LVKDQEALMkSVK | SEQ ID NO: 134 |
| | | | regulator | | | |
| 119 | RCOR1 | NP_055971.1 | Transcriptional | K418 | SVVQVkNFFVNYR | SEQ ID NO: 135 |
| | | | regulator | | | |
| 120 | RIZ | NP_036363.2 | Transcriptional | K1425 | kNQLVQKAILQKNK | SEQ ID NO: 136 |
| | | | regulator | | | |
| 121 | RIZ | NP_056950.2 | Transcriptional | K1431 | KNQLVQkAILQKNK | SEQ ID NO: 137 |
| | | | regulator | | | |
| 122 | RREB-1 | NP_002946.4 | Transcriptional | K1400 | LACDTCGkSFK | SEQ ID NO: 138 |
| | | | regulator | | | |
| 123 | SRFBP1 | NP_689759.2 | Transcriptional | K423 | EQQSNIAVFQGkK | SEQ ID NO: 139 |
| | | | regulator | | | |
| 124 | ZNF313 | NP_061153.1 | Transcriptional | K112 | SHVATCSkYQNYIMEGVK | SEQ ID NO: 140 |
| | | | regulator | | | |
| 125 | ZNF611 | NP_112234.2 | Transcriptional | K238 | SGkAFNCSSLLR | SEQ ID NO: 141 |
| | | | regulator | | | |
| 126 | EEF1G | NP_001395.1 | Transcriptional | K147 | ILGLLDAYLkTR | SEQ ID NO: 145 |
| | | | regulator | | | |
| 127 | EEF1G | NP_001395.1 | Transcriptional | K212 | LCEkMAQFDAK | SEQ ID NO: 146 |
| | | | regulator | | | |
| 128 | eEF-2 | NP_001952.1 | Transcriptional | K426 | VFSGLVSTGLkVR | SEQ ID NO: 147 |
| | | | regulator | | | |
| 129 | eEF-2 | NP_001952.1 | Transcriptional | K235 | QFAEMYVAkFAAK | SEQ ID NO: 148 |
| | | | regulator | | | |
| 130 | eIF3-eta | NP_003742.2 | Transcriptional | K729 | KYSkIFEQK | SEQ ID NO: 149 |
| | | | regulator | | | |
| 131 | eIF3S6IP | NP_057175.1 | Transcriptional | K549 | QIHkFEELNR | SEQ ID NO: 150 |
| | | | regulator | | | |
| 132 | eIF4B | NP_001408.2 | Transcriptional | K586 | KPEENPASkFSSASK | SEQ ID NO: 151 |
| | | | regulator | | | |
| 133 | eIF5A | NP_001961.1 | Transcriptional | K67 | VHLVGIDIFTGkK | SEQ ID NO: 152 |
| | | | regulator | | | |
| 134 | GSPT2 | NP_060564.2 | Transcriptional | K102 | GPTQPPTLPAGSGSNDETCTGAGYPQGkR | SEQ ID NO: 153 |
| | | | regulator | | | |
| 135 | RPL10 | NP_006004.1 | Transcriptional | K208 | GPLDkWR | SEQ ID NO: 154 |
| | | | regulator | | | |
| 136 | RPL10A | NP_009035.3 | Transcriptional | K62 | STPRPkFSVCVLGDQQHCDEAK | SEQ ID NO: 155 |
| | | | regulator | | | |
| 137 | RPL10L | NP_542784.1 | Transcriptional | K208 | GPLDkWR | SEQ ID NO: 156 |
| | | | regulator | | | |
| 138 | RPL11 | NP_000966.2 | Transcriptional | K52 | VLEQLTGQTPVFSkAR | SEQ ID NO: 157 |
| | | | regulator | | | |
| 139 | RPL12 | NP_000967.1 | Transcriptional | K40 | IGPLGLSPkK | SEQ ID NO: 158 |
| | | | regulator | | | |
| 140 | RLP13A | NP_036555.1 | Transcriptional | K25 | LAAIVAkQVLLGR | SEQ ID NO: 159 |
| | | | regulator | | | |
| 141 | RLL17 | NP_000976.1 | Transcriptional | K74 | CAQAkQWGWTQGR | SEQ ID NO: 160 |
| | | | regulator | | | |
| 142 | RPL18a | NP_000971.1 | Transcriptional | K76 | VkNFGIWLR | SEQ ID NO: 161 |
| | | | regulator | | | |
| 143 | RLP24 | NP_000977.1 | Transcriptional | K27 | TDGkVFQFLNAK | SEQ ID NO: 162 |
| | | | regulator | | | |
| 144 | RPL4 | NP_000959.2 | Transcriptional | K412 | KPAPEKkPAEK | SEQ ID NO: 163 |
| | | | regulator | | | |
| 145 | RPL5 | NP_000960.2 | Transcriptional | K220 | YLMEEDEDAYkK | SEQ ID NO: 164 |
| | | | regulator | | | |
| 146 | RPL7L1 | NP_940888.2 | Transcriptional | K42 | EQkKGKGLR | SEQ ID NO: 165 |
| | | | regulator | | | |
| 147 | RPLP2 | NP_000995.1 | Transcriptional | K21 | YVASYLLAALGGNSSPSAkDIK | SEQ ID NO: 166 |
| | | | regulator | | | |
| 148 | RPS17 | NP_001012.1 | Transcriptional | K19 | VIIEkYYTR | SEQ ID NO: 167 |
| | | | regulator | | | |
| 149 | RPS21 | NP_001015.1 | Transcriptional | K81 | ADGIVSkNF | SEQ ID NO: 168 |
| | | | regulator | | | |
| 150 | RPS5 | NP_001000.2 | Transcriptional | K94 | IVkHAFEIIHLLTGENPLQVLVNAIINSGPR | SEQ ID NO: 169 |
| | | | regulator | | | |
| 151 | RPS7 | NP_001002.1 | Transcriptional | K37 | IVKPNGEKPDEFESGISQALLELEMNSDLk | SEQ ID NO: 170 |
| | | | regulator | | | |
| 152 | RPS7 | NP_001002.1 | Transcriptional | K70 | AIIFVPVPQLkSFQK | SEQ ID NO: 171 |
| | | | regulator | | | |
| 153 | TUFM | NP_003312.3 | Transcriptional | K82 | TTLTAAITkILAEGGGAK | SEQ ID NO: 172 |
| | | | regulator | | | |
| 154 | DZIP3 | NP_055463.1 | Ubiquitin | K663 | QRKKkKTKNKK | SEQ ID NO: 173 |
| | | | conjugating | | | |
| | | | system | | | |
| 155 | FBXW2 | NP_036296.1 | Ubiquitin | K298 | SLLHSPGDYILLSADkYEIK | SEQ ID NO: 174 |
| | | | conjugating | | | |
| | | | system | | | |
| 156 | RFFL | NP_476519.1 | Ubiquitin | K105 | FRATAFQREELMKMk | SEQ ID NO: 175 |
| | | | conjugating | | | |
| | | | system | | | |
| 157 | SAE2 | NP_005490.1 | Ubiquitin | K253 | SYGYDPVkLFTK | SEQ ID NO: 176 |
| | | | conjugating | | | |
| | | | system | | | |
| 158 | SKP1A | NP_733779.1 | Ubiquitin | K163 | KENQWCEEk | SEQ ID NO: 177 |
| | | | conjugating | | | |
| | | | system | | | |
| 159 | UBE2N | NP_003339.1 | Ubiquitin | K92 | ICLDILkDKWSPALQIR | SEQ ID NO: 178 |
| | | | conjugating | | | |
| | | | system | | | |
| 160 | UBE2S | NP_055316.1 | Ubiquitin | K68 | LLLGkDFPASPPK | SEQ ID NO: 179 |
| | | | conjugating | | | |
| | | | system | | | |
| 161 | UCHL5 | NP_057068.1 | Ubiquitin | K158 | TSAkEEDAFHFVSYVPVNGR | SEQ ID NO: 180 |
| | | | conjugating | | | |
| | | | system | | | |
| 162 | ACTBL1 | NP_001004053.1 | Unknown function | K208 | DTDMNKKDkQK | SEQ ID NO: 182 |
| 163 | ADNP | NP_056154.1 | Unknown function | K1035 | NSSYGkVEGFWSK | SEQ ID NO: 183 |
| 164 | ANKRD21 | NP_778146.1 | Unknown function | K295 | kANLNVLDR | SEQ ID NO: 184 |
| 165 | API5 | NP_006586.1 | Unknown function | K448 | STVTLSWKPVQkVEIGQK | SEQ ID NO: 185 |
| 166 | APM2 | NP_006820.1 | Unknown function | K70 | TANQASDTFSGIGkK | SEQ ID NO: 186 |
| 167 | ARMC4 | NP_060546.2 | Unknown function | K891 | SFVGGLELIVNLLk | SEQ ID NO: 187 |
| 168 | BAT2D1 | NP_055987.1 | Unknown function | K27 | YATLSLFNTYkGK | SEQ ID NO: 188 |
| 169 | BCL9L | NP_872383.3 | Unknown function | K36 | GHCPPAPAkPMHPENK | SEQ ID NO: 190 |
| 170 | BOLA1 | NP_057158.1 | Unknown function | K131 | ENSQLDTSPPCLGGNkK | SEQ ID NO: 191 |
| 171 | BRD1 | NP_055392.1 | Unknown function | K519 | ENDEEMKAAkEK | SEQ ID NO: 192 |
| 172 | BUD13 | NP_116114.1 | Unknown function | K47 | PKPGGAGGkGMR | SEQ ID NO: 194 |
| 173 | BXDC1 | NP_115570.1 | Unknown function | K47 | NAMLIKGGNANATVTkVLKDVYALKK | SEQ ID NO: 195 |
| 174 | BXDC1 | NP_115570.1 | Unknown function | K56 | NAMLIKGGNANATVTKVLKDVYALkK | SEQ ID NO: 196 |
| 175 | C10orf119 | NP_079110.1 | Unknown function | K328 | LQHINPLLPACLNkEESK | SEQ ID NO: 197 |
| 176 | C10orf42 | NP_612366.1 | Unknown function | K332 | DAIAQAEMDLkR | SEQ ID NO: 198 |
| 177 | C10orf76 | NP_001002759.1 | Unknown function | K215 | SCSQLLLYELQSAVSEENkK | SEQ ID NO: 199 |
| 178 | C14orf106 | NP_060823.3 | Unknown function | K113 | ANYESPGkIFLR | SEQ ID NO: 202 |
| 179 | C16orf57 | NP_078874.2 | Unknown function | K258 | SGNkFFSMPLK | SEQ ID NO: 203 |
| 180 | C19orf43 | NP_076943.1 | Unknown function | K118 | GGPGSTLSFVGkR | SEQ ID NO: 204 |
| 181 | C1orf114 | NP_067002.1 | Unknown function | K465 | RKRMEkMARQQAVRER | SEQ ID NO: 205 |
| 182 | C1orf14 | NP_073593.2 | Unknown function | K69 | YLTNQkNSNSK | SEQ ID NO: 206 |
| 183 | C1orf49 | NP_073593.2 | Unknown function | K74 | YLTNQKNSNSkNDR | SEQ ID NO: 207 |
| 184 | C21orf33 | NP_004640.1 | Unknown function | K164 | VLkEFHQAGKPIGLCCIAPVLAAK | SEQ ID NO: 208 |
| 185 | C21orf70 | NP_478070.1 | Unknown function | K118 | EQWLQkIEAIK | SEQ ID NO: 209 |
| 186 | C6orf47 | NP_067007.3 | Unknown function | K153 | REkLLGWLR | SEQ ID NO: 210 |
| 187 | C8orf59 | XP_376783.1 | Unknown function | K21 | NVFHIASQkNFK | SEQ ID NO: 211 |
| 188 | C9orf39 | NP_060208.1 | Unknown function | K718 | kLNLDLAGLR | SEQ ID NO: 212 |
| 189 | CARF | NP_060102.1 | Unknown function | K184 | ASAQQENSSTCIGSAIkSESGNSAR | SEQ ID NO: 213 |
| 190 | CGI-115 | NP_057136.1 | Unknown function | K185 | kDFISVLR | SEQ ID NO: 215 |
| 191 | coitlin | NP_004636.1 | Unknown function | K405 | EENLFSWkGAK | SEQ ID NO: 216 |
| 192 | CT45-2 | NP_689795.2 | Unknown function | K140 | CVGQkYEK | SEQ ID NO: 217 |
| 193 | CTGLF1 | CAH72489.1 | Unknown function | K507 | kVMSSIVNDLANSIWEGSSQGQTK | SEQ ID NO: 218 |
| 194 | CTGLF1 | CAH72489.1 | Unknown function | K530 | KVMSSIVNDLANSIWEGSSQGQTk | SEQ ID NO: 219 |
| 195 | CWF19L1 | NP_060764.3 | Unknown function | K174 | CVGNFFNSSGEVDTkK | SEQ ID NO: 220 |
| 196 | DDX29 | NP_061903.2 | Unknown function | K87 | SILkVVINNK | SEQ ID NO: 221 |
| 197 | DDX29 | NP_061903.2 | Unknown function | K93 | SILKNNINNk | SEQ ID NO: 222 |
| 198 | DDX52 | NP_008941.2 | Unknown function | K131 | KTSGkLENLR | SEQ ID NO: 223 |
| 199 | DDX55 | NP_065987.1 | Unknown function | K523 | KFIKNKAWSkQK | SEQ ID NO: 224 |
| 200 | DOCK7 | NP_212132.2 | Unknown function | K1931 | AHGELHEQFkR | SEQ ID NO: 225 |
| 201 | EVI5L | NP_660288.1 | Unknown function | K484 | EMQDkVLDMEK | SEQ ID NO: 226 |
| 202 | FAM21C | NP_056077.1 | Unknown function | K796 | TkTVLSLFDEEEDK | SEQ ID NO: 227 |
| 203 | FIGNL1 | NP_071399.2 | Unknown function | K339 | GILGkFVPPIPK | SEQ ID NO: 228 |
| 204 | FLJ11151 | NP_060810.1 | Unknown function | K109 | TEQTEDLkR | SEQ ID NO: 229 |
| 205 | FLJ14351 | AAH93743.1 | Unknown function | K105 | LHIRSINkGGQLR | SEQ ID NO: 230 |
| 206 | FLJ22052 | NP_060395.4 | Unknown function | K274 | KNMNWKQLPk | SEQ ID NO: 231 |
| 207 | CCDC86 | NP_077003.1 | Ubiquitin | K212 | GQHEPSKPPPAGETVTGGFGAkKR | SEQ ID NO: 232 |
| 208 | FLJ22965 | NP_071384.1 | Unknown function | K222 | GTLIDNQFk | SEQ ID NO: 233 |
| 209 | FLJ31033 | XP_936057.1 | Unknown function | K945 | ITkKNPKKAKELERKKVYR | SEQ ID NO: 235 |
| 210 | FLJ31033 | XP_936057.1 | Unknown function | K949 | ITKKNPkKAEKLERKKVYR | SEQ ID NO: 236 |
| 211 | FLJ31033 | XP_936057.1 | Unknown function | K950 | ITKKNPKkAEKLERKKVYR | SEQ ID NO: 237 |
| 212 | FLJ31033 | XP_936057.1 | Unknown function | K953 | ITKKNPKKAEkLERKKVYR | SEQ ID NO: 238 |
| 213 | FLJ31033 | XP_936057.1 | Unknown function | K957 | ITKKNPKKAEKLERkKVYR | SEQ ID NO: 239 |
| 214 | FLJ35843 | NP_689804.1 | Unknown function | K103 | LLLQTkEDYEALIKSLK | SEQ ID NO: 240 |
| 215 | FLJ35843 | NP_669804.1 | Unknown function | K111 | LLLQTKEDYEALIkSLK | SEQ ID NO: 241 |
| 216 | FLJ38343 | NP_777591.1 | Unknown function | K20 | AEEkLESYR | SEQ ID NO: 242 |
| 217 | FLJ43950 | XP_294521.4 | Unknown function | K50 | RKkGGTYKDRK | SEQ ID NO: 243 |
| 218 | FLJ43950 | XP_294521.4 | Unknown function | K58 | RKKGGTYKDRk | SEQ ID NO: 244 |
| 219 | HCA66 | NP_060898.2 | Unknown function | K339 | FTkKSNSGFLRGKR | SEQ ID NO: 245 |
| 220 | HCA66 | NP_060898.2 | Unknown function | K340 | FTKkSNSGFLRGKR | SEQ ID NO: 246 |
| 221 | HDDC3 | NP_940929.1 | Unknown function | K97 | LVEEVTDDKTLPkLER | SEQ ID NO: 247 |
| 222 | HDHD2 | NP_115500.1 | Unknown function | K179 | ATVVGkPEKTFFLEALR | SEQ ID NO: 249 |
| 223 | HDHD3 | NP_113496.1 | Unknown function | K15 | LLTWDVkDTLLR | SEQ ID NO: 250 |
| 224 | HEATR1 | NP_060542.4 | Unknown function | K1679 | LLCkNFGAENPDPFVPVLNTAVK | SEQ ID NO: 251 |
| 225 | HGR8 | NP_057342.1 | Unknown function | K401 | SINNYNPkDFDWNLK | SEQ ID NO: 252 |
| 226 | HRBL | NP_006067.3 | Unknown function | K173 | GSASTPVQGSIPEGkPLR | SEQ ID NO: 253 |
| 227 | HRBL | NP_006067.3 | Unknown function | K7 | kGPGPGGGVSGGKAEAEAASEVWCR | SEQ ID NO: 254 |
| 228 | IFIT5 | NP_036552.1 | Unknown function | K150 | GWALLkFGGK | SEQ ID NO: 255 |
| 229 | IQCA | NP_079002.3 | Unknown function | K471 | KKDkKGKKGK | SEQ ID NO: 256 |
| 230 | IQCA | NP_079002.3 | Unknown function | K472 | KKDKkGKKGK | SEQ ID NO: 257 |
| 231 | IQCA | NP_079002.3 | Unknown function | K474 | KKDKKGkKGK | SEQ ID NO: 258 |
| 232 | ISOC2 | NP_078986.1 | Unknown function | K201 | LIkEPAPDSGLLGLFQGQNSLLH | SEQ ID NO: 259 |
| 233 | ISOC2 | NP_078986.1 | Unknown function | K26 | VLPGSSVLFLCDMQEkFR | SEQ ID NO: 260 |
| 234 | KIAA0355 | NP_055501.2 | Unknown function | K1054 | GFkAFPGKGER | SEQ ID NO: 261 |
| 235 | KIAA0355 | NP_055501.2 | Unknown function | K1059 | GFKAFPGkGER | SEQ ID NO: 262 |
| 236 | KIAA0794 | XP_087353.6 | Unknown function | K51 | APIPQkQEILVEPEPLFGAPK | SEQ ID NO: 263 |
| 237 | KIAA1196 | NP_065764.1 | Unknown function | K834 | EkKKNLAGGKKR | SEQ ID NO: 266 |
| 238 | KIAA1196 | NP_065764.1 | Unknown function | K836 | EKKkNLAGGKKR | SEQ ID NO: 267 |
| 239 | KIAA1196 | NP_065764.1 | Unknown function | K843 | EKKKNLAGGKkR | SEQ ID NO: 268 |
| 240 | KIAA1407 | NP_065868.1 | Unknown function | K177 | kENQDKKQQKDPR | SEQ ID NO: 269 |
| 241 | KIAA1407 | NP_065868.1 | Unknown function | K182 | KENQDkKQQKDPR | SEQ ID NO: 270 |
| 242 | KIAA1429 | NP_056311.2 | Unknown function | K166 | NPKHADGEk | SEQ ID NO: 271 |
| 243 | KIAA1458 | XP_044434.3 | Unknown function | K459 | MQPQASAIPSPGkFR | SEQ ID NO: 272 |
| 244 | LANP-L | NP_112182.1 | Unknown function | K254 | EEIQDEEDDDDYVEEGEEEEEEEEGGLRGE | SEQ ID NO: 282 |
| | | | | | kR | |
| 245 | LARP | NP_056130.2 | Unknown function | K933 | NLDIDPkLQEYLGK | SEQ ID NO: 283 |
| 246 | LENG8 | NP_443157.1 | Unknown function | K400 | GNSFTkFGNR | SEQ ID NO: 284 |
| 247 | LENG8 | NP_443157.1 | Unknown function | K511 | ELKKQkRAAR | SEQ ID NO: 285 |
| 248 | LEREPO4 | NP_060941.1 | Unknown function | K27 | IIEDkTFGLK | SEQ ID NO: 286 |
| 249 | LOC121006 | XP_931911.1 | Unknown function | K632 | EELTkQVKSHQLVKSLSRVAK | SEQ ID NO: 287 |
| 250 | LOC121006 | XP_931911.1 | Unknown function | K648 | EELTKQVKSHQLVKSLSRVAk | SEQ ID NO: 288 |
| 251 | LOC124245 | NP_653205.2 | Unknown function | K783 | EEKRKRDSSTQPPKSAk | SEQ ID NO: 289 |
| 252 | LOC211416 | NP_037398.1 | Unknown function | K860 | MKAPAKKPLIkR | SEQ ID NO: 290 |
| 253 | LOC388160 | XP_370894.1 | Unknown function | K39 | LTMkTDSLMEEK | SEQ ID NO: 291 |
| 254 | LOC56902 | NP_064528.1 | Unknown function | K72 | RPVFPPLCGDGLLSGkEETR | SEQ ID NO: 295 |
| 255 | LOC644535 | XP_293911.5 | Unknown function | K631 | VTYLGkELLR | SEQ ID NO: 297 |
| 256 | LOC650200 | XP_944377.1 | Unknown function | K155 | KVAQkLLNGQRK | SEQ ID NO: 298 |
| 257 | LOC650200 | XP_944377.1 | Unknown function | K162 | KVAQKLLNGQRk | SEQ ID NO: 299 |
| 258 | LUZP1 | NP_361013.2 | Unknown function | K297 | NQEDNKVKDLNQEIEkLK | SEQ ID NO: 300 |
| 259 | MGC16597 | XP_375500.1 | Unknown function | K64 | EkKKVNCKPK | SEQ ID NO: 301 |
| 260 | MGC2731 | NP_076973.1 | Unknown function | K203 | SQPNHTPAGPPGPSSNPVSNGkETR | SEQ ID NO: 302 |
| 261 | MGC4606 | NP_078792.1 | Unknown function | K203 | LDkVLSDMKR | SEQ ID NO: 303 |
| 262 | MGC4606 | NP_078792.1 | Unknown function | K209 | LDKVLSDMkR | SEQ ID NO: 304 |
| 263 | MPHOSPH8 | NP_059990.2 | Unknown function | K169 | SPDDLkKKKAKAGK | SEQ ID NO: 305 |
| 264 | MPHOSPH8 | NP_059990.2 | Unknown function | K170 | SPDDLKkKKAKAGK | SEQ ID NO: 306 |
| 265 | MPHOSPH8 | NP_059990.2 | Unknown function | K172 | SPDDLKKKkAKAGK | SEQ ID NO: 307 |
| 266 | MPHOSPH8 | NP_059990.2 | Unknown function | K177 | SPDDLKKKKAKAGk | SEQ ID NO: 308 |
| 267 | NALP4 | NP_604393.1 | Unknown function | K611 | LCFSVQNVFkK | SEQ ID NO: 309 |
| 268 | NGRN | NP_057729.1 | Unknown function | K63 | FLPTLEQKLkQDQKVLKK | SEQ ID NO: 310 |
| 269 | NIP30 | NP_079222.1 | Unknown function | K151 | FSQAkLLAGAVK | SEQ ID NO: 311 |
| 270 | N-PAC | NP_115958.2 | Unknown function | K72 | EEMIkINKGKR | SEQ ID NO: 312 |
| 271 | N-PAC | NP_115958.2 | Unknown function | K77 | EEMIKINKGkR | SEQ ID NO: 313 |
| 272 | NSUN2 | NP_060225.4 | Unknown function | K28 | LQQQQRPEDAEDGAEGGGkR | SEQ ID NO: 315 |
| 273 | NSUN2 | NP_060225.4 | Unknown function | K586 | VINTGIkVWCR | SEQ ID NO: 316 |
| 274 | NT5C2L1 | NP_689942.2 | Unknown function | K455 | TAGYYPNPPLVLSSDETLISk | SEQ ID NO: 317 |
| 275 | OLFML2A | NP_872293.1 | Unknown function | K6 | DkAGKDTARGK | SEQ ID NO: 318 |
| 276 | OLFML2A | NP_872293.1 | Unknown function | K9 | DKAGkDTARGK | SEQ ID NO: 319 |
| 277 | PCNP | NP_065090.1 | Unknown function | K152 | GkHGFSDNQK | SEQ ID NO: 320 |
| 278 | PCNP | NP_065090.1 | Unknown function | K64 | SAEEEAADLPTkPTK | SEQ ID NO: 321 |
| 279 | PCNP | NP_065090.1 | Unknown function | K70 | ISkFGFAIGSQTTK | SEQ ID NO: 322 |
| 280 | PHF15 | NP_056103.4 | Unknown function | K32 | CSkLPSSTKSGWPR | SEQ ID NO: 326 |
| 281 | PHF15 | NP_056103.4 | Unknown function | K38 | CSKLPSSTkSGWPR | SEQ ID NO: 327 |
| 282 | POTE14 | NP_001005356.1 | Unknown function | K171 | DTDMNKKDkQK | SEQ ID NO: 331 |
| 283 | PSPC1 | NP_060752.2 | Unknown function | K99 | LFVGNLPTDITEEDFkR | SEQ ID NO: 332 |
| 284 | Q8WVJ2 | NP_860309.1 | Unknown function | K157 | GGPDFSNLEk | SEQ ID NO: 333 |
| 285 | RP11- | NP_149102.2 | Unknown function | K384 | NkKRRKKR | SEQ ID NO: 335 |
| | 298P3.3 | | | | | |
| 286 | RP11- | NP_149102.2 | Unknown function | K385 | NKkRRKKR | SEQ ID NO: 336 |
| | 298P3.3 | | | | | |
| 287 | RP11- | NP_149102.2 | Unknown function | K389 | NKKRRKkR | SEQ ID NO: 337 |
| | 298P3.3 | | | | | |
| 288 | RSHL2 | NP_114130.3 | Unknown function | K227 | QREARkRALARK | SEQ ID NO: 338 |
| 289 | RSHL2 | NP_114130.3 | Unknown function | K233 | QREARKRALARk | SEQ ID NO: 339 |
| 290 | SAMHD1 | NP_056289.2 | Unknown function | K332 | DCHHLGIQNNFDYkR | SEQ ID NO: 343 |
| 291 | SCRN1 | NP_055581.2 | Unknown function | K306 | SIFkPFIFVDDVK | SEQ ID NO: 344 |
| 292 | SH3BP1 | NP_061830.3 | Unknown function | K142 | HkKSLQKLVSDWNTLKSR | SEQ ID NO: 345 |
| 293 | SH3BP1 | NP_061830.3 | Unknown function | K143 | HKkSLQKLVSDWNTLKSR | SEQ ID NO: 346 |
| 294 | SH3BP1 | NP_061830.3 | Unknown function | K147 | HKKSLQkLVSDWNTLKSR | SEQ ID NO: 347 |
| 295 | SmcX | NP_004178.2 | Unknown function | K205 | QSVQPSkFNSYGR | SEQ ID NO: 348 |
| 296 | SPAG7 | NP_004881.1 | Unknown function | K197 | TYGCVPVANkR | SEQ ID NO: 352 |
| 297 | TDRD6 | NP_001010870.1 | Unknown function | K403 | TLILGkAVNAK | SEQ ID NO: 353 |
| 298 | TPD52L2 | NP_003279.2 | Unknown function | K165 | NSATFkSFEDR | SEQ ID NO: 356 |
| 299 | UBAP2 | NP_060919.2 | Unknown function | K126 | SEkESSRGRGNNNRK | SEQ ID NO: 357 |
| 300 | URB | NP_955805.1 | Unknown function | K232 | LEKGkFGMVLLKK | SEQ ID NO: 360 |
| 301 | UTP14A | NP_006640.2 | Unknown function | K56 | LLEAISSLDGkNR | SEQ ID NO: 361 |
| 302 | VPRBP | NP_055518.1 | Unknown function | K701 | ISSIGkFISGTPR | SEQ ID NO: 363 |
| 303 | WDR33 | NP_060853.3 | Unknown function | K46 | RPDFAQQQAMQQLTFDGkR | SEQ ID NO: 364 |
| 304 | WDR67 | NP_663622.1 | Unknown function | K403 | kNELPDGLNKKR | SEQ ID NO: 365 |
| 305 | WDR67 | NP_663622.1 | Unknown function | K413 | KNELPDGLNKkR | SEQ ID NO: 366 |
| 306 | WDR75 | NP_115544.1 | Unknown function | K466 | SEQPTLVTASkDGYFK | SEQ ID NO: 367 |
| 307 | WHDC1L1 | XP_370838.3 | Unknown function | K7 | MEASVPkYKKHLPQLGMQK | SEQ ID NO: 368 |
| 308 | WHDC1L1 | XP_370838.3 | Unknown function | K9 | MEASVPKYkKHLPQLGMQK | SEQ ID NO: 369 |
| 309 | WHSC1L1 | NP_075447.1 | Unknown function | K790 | KFPTAIFESkGFR | SEQ ID NO: 370 |
| 310 | ZAP3 | XP_085151.9 | Unknown function | K1890 | VLSLDDYFITEVEKEEKDPDSGkK | SEQ ID NO: 372 |
| 311 | ZCCHC14 | NP_055959.1 | Unknown function | K324 | QLSMEkFLSLTEEDLNK | SEQ ID NO: 377 |
| 312 | ZNF198 | NP_003444.1 | Unknown function | K1280 | YQVSSLCGTDNEDKITTGkR | SEQ ID NO: 378 |
| 313 | ZNF403 | NP_079111.1 | Unknown function | K537 | KKkKSKILK | SEQ ID NO: 379 |
| 314 | ZNF403 | NP_079111.1 | Unknown function | K538 | KKKkSKILK | SEQ ID NO: 380 |
| 315 | ZNF512 | NP_115810.2 | Unknown function | K18 | LGAVPATSGPTTFkQQR | SEQ ID NO: 381 |
| 316 | ZNF650 | NP_742067.2 | Unknown function | K21 | kVTAAEKK | SEQ ID NO: 382 |
| 317 | ZNF650 | NP_742067.2 | Unknown function | K27 | KVTAAEkK | SEQ ID NO: 383 |
| 318 | ARCN1 | NP_001646.2 | Vesicle protein | K351 | LFTAESLIGLKNPEkSFPVNSDVGVLK | SEQ ID NO: 386 |
| 319 | GLG1 | NP_036333.2 | Vesicle protein | K747 | CARGVTHFQLVQMkDFR | SEQ ID NO: 387 |
| 320 | NSFL1C | NP_057227.2 | Vesicle protein | K251 | GAFkAFTGEGQK | SEQ ID NO: 388 |
| 321 | SNX1 | NP_003090.2 | Vesicle protein | K237 | VKVGKEDSSSAEFLEkR | SEQ ID NO: 389 |
| 322 | TAP | NP_003706.1 | Vesicle protein | K815 | LQTEkQELLQK | SEQ ID NO: 390 |
| 323 | TOLLIP | NP_061882.2 | Vesicle protein | K235 | kAIQDMF | SEQ ID NO: 391 |

One of skill in the art will appreciate that, in many instances the utility of the instant invention is best understood in conjunction with an appreciation of the many biological roles and significance of the various target signaling proteins/polypeptides of the invention. The foregoing is illustrated in the following paragraphs summarizing the knowledge in the art relevant to a few non-limiting representative peptides containing selected acetylation sites according to the invention.

FNBP3, phosphorylated at K330, K333 and K334, is among the proteins listed in this patent. This protein has potential diagnostic and/or therapeutic implications based on the following findings. Abnormal protein binding of FNBP3 may cause Huntington disease (Hum Mol Genet 7: 1463-74 (1998)). Abnormal protein binding of FNBP3 may cause abnormal mRNA metabolic process associated with Huntington disease (Hum Mol Genet 9: 2175-82 (2000)). Decreased expression of FNBP3 in neurons correlates with Huntington disease (Hum Mol Genet 9: 2175-82 (2000)). (PhosphoSite®, Cell Signaling Technology (Danvers, Mass.), Human PSD™, Biobase Corporation, (Beverly, Mass.)).

ARPC2, phosphorylated at K295, is among the proteins listed in this patent. ARPC2, Actin related protein 2-3 complex subunit 2 (34kDa), component of the Arp2-3 complex, which is involved in assembly of the actin cytoskeleton, interacts directly with ARPC4, possibly as an early intermediate in Arp2-3 complex formation. (PhosphoSite®, Cell Signaling Technology (Danvers, Mass.), Human PSD™, Biobase Corporation, (Beverly, Mass.)).

gelsolin, phosphorylated at K648, is among the proteins listed in this patent. This protein has potential diagnostic and/or therapeutic implications based on the following findings. Decreased expression of GSN protein correlates with bladder neoplasms (Cancer Res 55: 3228-32. (1995)). Decreased expression of GSN mRNA may correlate with breast neoplasms (Int J Cancer 81: 930-8 (1999)). Abnormal processing of GSN causes abnormal neurons structure associated with familial amyloid neuropathies (JBC 273: 16319-24. (1998)). Abnormal folding of GSN causes abnormal neurons structure associated with familial amyloid neuropathies (JBC 273: 16319-24. (1998)). Increased expression of GSN protein correlates with increased occurrence of recurrence associated with bladder neoplasms (Cancer 95: 1247-57. (2002)). Missense mutation in the GSN gene causes amyloidosis associated with familial amyloid neuropathies (Chem Biol 6: 293-304. (1999)). Decreased expression of GSN protein may cause decreased mitotic G2 checkpoint associated with bladder neoplasms (Exp Cell Res 251: 224-33. (1999)). Abnormal processing of GSN causes abnormal neurons structure associated with familial amyloid neuropathies (J Biol Chem 273: 16319-24. (1998)). Increased expression of GSN protein may prevent abnormal actin filament polymerization associated with cystic fibrosis (Biochemistry Usa 36: 9637-41. (1997)). Decreased expression of GSN protein may cause bladder neoplasms (Cancer Res 55: 3228-32. (1995)). Increased expression of GSN protein correlates with increased occurrence of recurrence associated with lung neoplasms (Cancer 85: 47-57. (1999)). Decreased stability of GSN may cause amyloidosis associated with familial amyloid neuropathies (J Mol Biol 334: 119-27. (2003)). Missense mutation in the GSN gene causes familial amyloid neuropathies (FEBS Lett 276: 75-7 (1990)). Abnormal proteolysis of GSN causes amyloidosis associated with familial amyloid neuropathies (Proc Natl Acad Sci USA 97: 10706-11 (2000)). Increased proteolysis of GSN may cause amyloidosis associated with familial amyloid neuropathies (J Mol Biol 334: 119-27. (2003)). Decreased expression of GSN mRNA may correlate with malignant form of prostatic neoplasms (Int J Cancer 112: 231-8 (2004)). Decreased expression of GSN protein correlates with non-familial form of breast neoplasms (Cancer Res 56: 4841-5. (1996)). Decreased expression of GSN protein may cause decreased mitotic G2 checkpoint associated with stomach neoplasms (Exp Cell Res 251: 224-33. (1999)). Decreased stability of GSN causes amyloidosis associated with familial amyloid neuropathies (PNAS 97: 10706-11. (2000)). Increased expression of GSN mRNA may prevent non-small-cell lung carcinoma associated with lung neoplasms (Cancer Res 60: 1129-38. (2000)). Mutation in the GSN gene causes familial amyloidosis (Nat Struct Biol 9: 112-6. (2002)). Decreased expression of GSN protein correlates with non-small-cell lung carcinoma associated with lung neoplasms (Cancer Res 58: 322-7. (1998)). Decreased expression of GSN mRNA may correlate with increased severity of breast neoplasms associated with carcinoma (Int J Cancer 112: 231-8 (2004)). Decreased stability of GSN causes familial amyloidosis (Nat Struct Biol 9: 112-6. (2002)). Abnormal proteolysis of GSN causes familial amyloidosis (Nat Struct Biol 9: 112-6. (2002)). Abnormal proteolysis of GSN causes amyloidosis associated with familial amyloid neuropathies (Proc Natl Acad Sci USA 97: 10706-11 (2000)). Decreased expression of GSN protein correlates with abnormal actin filament organization associated with breast neoplasms (Cancer Res 56: 4841-5. (1996)). Increased proteolysis of GSN causes amyloidosis associated with familial amyloid neuropathies (FEBS Lett 335: 119-23. (1993)). Increased expression of GSN protein may prevent abnormal actin filament polymerization associated with cystic fibrosis (Biochemistry 36: 9637-41. (1997)). Increased proteolysis of GSN causes amyloidosis associated with familial amyloid neuropathies (Chem Biol 6: 293-304. (1999)). Increased expression of GSN protein correlates with advanced stage or high grade form of bladder neoplasms (Cancer 95: 1247-57. (2002)). Decreased expression of GSN mRNA may correlate with malignant form of breast neoplasms (Int J Cancer 112: 231-8 (2004)). Decreased expression of GSN protein correlates with carcinoma tumors associated with breast neoplasms (Cancer Res 56: 4841-5. (1996)). Decreased calcium ion binding of GSN may cause amyloidosis associated with familial amyloid neuropathies (J Mol Biol 334: 119-27. (2003)). Decreased expression of GSN protein correlates with non-small-cell lung carcinoma (Cancer Res 58: 322-7. (1998)). Increased expression of GSN mRNA may prevent increased cell proliferation associated with breast neoplasms (Mol Med 6: 849-66. (2000)). Increased expression of GSN protein may prevent decreased lung function associated with cystic fibrosis (Science 263: 969-71 (1994)). Decreased expression of GSN mRNA may correlate with increased severity of prostatic neoplasms associated with carcinoma (Int J Cancer 112: 231-8 (2004)). Abnormal proteolysis of GSN causes amyloidosis associated with familial amyloid neuropathies (PNAS 97: 10706-11 (2000)). Increased expression of GSN mRNA may prevent decreased cell differentiation associated with lung neoplasms (Cancer Res 60: 1129-38. (2000)). Decreased stability of GSN causes amyloidosis associated with familial amyloid neuropathies (Proc Natl Acad Sci USA 97: 10706-11 (2000)). Increased expression of GSN protein may prevent increased protein kinase C activation associated with lung neoplasms (Br J Cancer 88: 606-12. (2003)). Increased expression of GSN mRNA may prevent decreased cell differentiation associated with non-small-cell lung carcinoma (Cancer Res 60: 1129-38. (2000)). Decreased expression of GSN protein correlates with invasive form of breast neoplasms (Cancer Res 56: 4841-5. (1996)). Decreased calcium ion binding of GSN causes familial amyloidosis (Nat Struct Biol 9: 112-6. (2002)). Increased expression of GSN protein correlates with increased occurrence of recurrence associated with non-small-cell lung carcinoma (Cancer 85: 47-57. (1999)). Increased expression of GSN in serum correlates with amyloidosis associated with familial amyloid neuropathies (FEBS Lett 406: 49-55 (1997)). Decreased expression of GSN mRNA may correlate with breast neoplasms (Exp Cell Res 276: 328-36. (2002)). Increased expression of GSN protein may prevent abnormal actin filament polymerization associated with cystic fibrosis (Science 263: 969-71 (1994)). Increased expression of GSN protein correlates with carcinoma tumors associated with bladder neoplasms (Cancer 95: 1247-57. (2002)). Abnormal folding of GSN causes abnormal neurons structure associated with familial amyloid neuropathies (J Biol Chem 273: 16319-24. (1998)). Increased expression of GSN mRNA may prevent decreased cell differentiation associated with breast neoplasms (Mol Med 6: 849-66. (2000)). Mutation in the GSN gene may correlate with familial form of amyloidosis (Genomics 13: 898-901 (1992)). Increased expression of GSN protein may prevent increased cell proliferation associated with lung neoplasms (Br J Cancer 88: 606-12. (2003)). Decreased stability of GSN causes amyloidosis associated with familial amyloid neuropathies (Proc Natl Acad Sci USA 97: 10706-11 (2000)). Decreased androgen receptor binding of GSN may prevent abnormal androgen receptor signaling pathway associated with prostatic neoplasms (Cancer Res 63: 4888-94 (2003)). Increased expression of GSN in cerebrospinal fluid correlates with familial amyloidosis (Hum Mol Genet 5: 1237-43. (1996)). Decreased actin binding of GSN causes amyloidosis associated with familial amyloid neuropathies (FEBS Lett 335: 119-23. (1993)). Abnormal proteolysis of GSN causes amyloidosis associated with familial amyloid neuropathies (Hum Mol Genet 3: 2223-9. (1994)). (PhosphoSite®, Cell Signaling Technology (Danvers, Mass.), Human PSD™, Biobase Corporation, (Beverly, Mass.)).

USH1C, phosphorylated at K451, is among the proteins listed in this patent. This protein has potential diagnostic and/or therapeutic implications based on the following findings. Splice site mutation in the USH1C gene correlates with deafness (Hum Genet 116: 225-7 (2005)). Deletion mutation in the USH1C gene causes blindness associated with sensorineural hearing loss (Nat Genet 26: 56-60 (2000)). Mutation in the USH1C gene correlates with retinal degeneration (Nat Genet 26: 51-55 (2000)). Deletion mutation in the USH1C gene causes autoimmune diseases associated with gastrointestinal diseases (Nat Genet 26: 56-60 (2000)). Splice site mutation in the USH1C gene correlates with autosomal recessive form of sensorineural hearing loss (Nat Genet 26: 51-55 (2000)). Decreased protein binding of USH1C may cause sensorineural hearing loss (Proc Natl Acad Sci USA 99: 14946-51. (2002)). Frameshift mutation in the USH1C gene correlates with autosomal recessive form of sensorineural hearing toss (Nat Genet 26: 51-55 (2000)). Frameshift mutation in the USH1C gene correlates with blindness associated with sensorineural hearing loss (Nat Genet 26: 51-55 (2000)). Decreased protein binding of USH1C may cause sensorineural hearing loss (PNAS 99: 14946-51. (2002)). Deletion mutation in the USH1C gene causes autosomal recessive form of sensorineural hearing loss (Nat Genet 26: 56-60 (2000)). Increased presence of USH1C autoimmune antibody correlates with colonic neoplasms (Int J Cancer 76: 652-8 (1998)). Splice site mutation in the USH1C gene may cause multiple abnormalities (Hum Genet 110: 527-31. (2002)). Increased expression of USH1C protein may prevent decreased cell cycle arrest associated with colonic neoplasms (Cancer Lett 211: 209-18 (2004)). Deletion mutation in the USH1C gene causes early onset form of retinitis pigmentosa (Nat Genet 26: 56-60 (2000)). Mutation in the USH1C gene causes autosomal recessive form of deafness (Hum Genet 111: 26-30. (2002)). Increased presence of USH1C autoimmune antibody correlates with kidney diseases associated with gastrointestinal diseases (Gastroenterology 117: 823-30 (1999)). Deletion mutation in the USH1C gene causes kidney diseases (Nat Genet 26: 56-60 (2000)). Mutation in the USH1C gene correlates with multiple abnormalities (Hum Genet 116: 292-9 (2005)). Splice site mutation in the USH gene correlates with blindness associated with sensorineural hearing loss (Nat Genet 26: 51-55 (2000)). USH epitope correlates with colonic neoplasms (Int J Cancer 76: 652-8 (1998)). Deletion mutation in the USH1C gene causes early onset form of hyperinsulinism (Nat Genet 26: 56-60 (2000)). Splice site mutation in the USH1C gene may cause autosomal recessive form of deafness (Hum Genet 110: 527-31. (2002)). Decreased protein binding of USH may cause sensorineural hearing loss (Proc Natl Acad Sci USA 99: 14946-51. (2002)). (PhosphoSite®, Cell Signaling Technology (Danvers, Mass.), Human PSD™, Biobase Corporation, (Beverly, Mass.)).

HEMH, phosphorylated at K415, is among the proteins listed in this patent. This protein has potential diagnostic and/or therapeutic implications based on the following findings. Decreased ferrochelatase activity of FECH causes liver failure associated with erythrohepatic porphyria (J Clin Invest 102: 107-14. (1998)). Nonsense mutation in the FECH gene correlates with liver failure associated with erythrohepatic porphyria (Hum Genet 93: 711-3. (1994)). Decreased expression of FECH protein correlates with liver failure associated with erythrohepatic porphyria (Lancet 343: 1394-6. (1994)). Mutation in the FECH gene causes autosomal recessive form of erythrohepatic porphyria (Lancet 343: 1394-6. (1994)). Splice site mutation in the FECH gene correlates with non-familial form of erythrohepatic porphyria (Biochim Biophys Acta 1316: 149-52. (1996)). (PhosphoSite®, Cell Signaling Technology (Danvers, Mass.), Human PSD™, Biobase Corporation, (Beverly, Mass.)).

PDA6, phosphorylated at K241 and K102, is among the proteins listed in this patent. PDA6, Protein disulfide isomerase family A member 6, binds integrin beta 3 (ITGB3) and plays a role in platelet activation and protein folding, induces alpha-granule secretion. (PhosphoSite®, Cell Signaling Technology (Danvers, Mass.), Human PSD™, Biobase Corporation, (Beverly, Mass.)).

SLC25A5, phosphorylated at K96, is among the proteins listed in this patent. This protein has potential diagnostic and/or therapeutic implications based on the following findings. Decreased expression of SLC25A5 in heart correlates with dilated cardiomyopathy (Mol Cell Biochem 174: 261-9 (1997)). (PhosphoSite®, Cell Signaling Technology (Danvers, Mass.), Human PSD™, Biobase Corporation, (Beverly, Mass.)).

DDX6, phosphorylated at K62, is among the proteins listed in this patent. This protein has potential diagnostic and/or therapeutic implications based on the following findings. Increased translation factor activity, nucleic acid binding of DDX6 may cause colorectal neoplasms (Carcinogenesis 22: 1965-70. (2001)). Translocation of the DDX6 gene causes malignant form of neoplasms (Br J Cancer 80: 914-7. (1999)). Increased expression of DDX6 mRNA correlates with colorectal neoplasms (Br J Cancer 80: 914-7. (1999)). Translocation of the DDX6 gene may correlate with B-cell lymphoma (Nucleic Acids Res 20: 1967-72 (1992)). Increased expression of DDX6 protein causes malignant form of neoplasms (Br J Cancer 80: 914-7. (1999)). Translocation of the DDX6 gene may correlate with diffuse large-cell lymphoma (Nucleic Acids Res 20: 1967-72 (1992)). (PhosphoSite®, Cell Signaling Technology (Danvers, Mass.), Human PSD™, Biobase Corporation, (Beverly, Mass.)).

ELAC2, phosphorylated at K152, is among the proteins listed in this patent. This protein has potential diagnostic and/or therapeutic implications based on the following findings. Nonsense mutation in the ELAC2 gene may correlate with increased occurrence of carcinoma associated with prostatic neoplasms (Cancer Res 61: 6494-9. (2001)). Missense mutation in the ELAC2 gene correlates with increased occurrence of familial form of prostatic neoplasms (Nat Genet 27: 172-80. (2001)). Missense mutation in the ELAC2 gene may correlate with increased occurrence of non-familial form of prostatic neoplasms (Am J Hum Genet 67: 1014-9 (2000)). Missense mutation in the ELAC2 gene correlates with increased occurrence of carcinoma associated with prostatic neoplasms (Nat Genet 27: 172-80. (2001)). Frameshift mutation in the ELAC2 gene may correlate with increased occurrence of carcinoma associated with prostatic neoplasms (Nat Genet 27: 172-80. (2001)). Missense mutation in the ELAC2 gene may correlate with increased incidence of benign form of prostatic hyperplasia (Cancer Res 61: 6038-41. (2001)). Frameshift mutation in the ELAC2 gene may correlate with increased occurrence of familial form of prostatic neoplasms (Nat Genet 27: 172-80. (2001)). (PhosphoSite®, Cell Signaling Technology (Danvers, Mass.), Human PSD™, Biobase Corporation, (Beverly, Mass.)).

KHSRP, phosphorylated at K109, is among the proteins listed in this patent. KHSRP, KH-type splicing regulatory protein, a pre-mRNA splicing factor and putative transcription factor that binds AU-specific RNA, involved in RNA splicing and mRNA destabilization; corresponding gene is upregulated in chronic fatigue syndrome. (PhosphoSite®, Cell Signaling Technology (Danvers, Mass.), Human PSD™, Biobase Corporation, (Beverly, Mass.)).

LRPPRC, phosphorylated at K187, is among the proteins listed in this patent. This protein has potential diagnostic and/or therapeutic implications based on the following findings. Missense mutation in the LRPPRC gene causes Leigh disease (PNAS 100: 605-10. (2003)). Missense mutation in the LRPPRC gene causes Leigh disease (Proc Natl Acad Sci USA 100: 605-10. (2003)). Missense mutation in the LRPPRC gene causes Leigh disease (Proc Natl Acad Sci USA 100: 605-10. (2003)). (PhosphoSite®, Cell Signaling Technology (Danvers, Mass.), Human PSD™, Biobase Corporation, (Beverly, Mass.)).

NSAP1, phosphorylated at K623, is among the proteins listed in ‘ this patent. NSAP1, Synaptotagmin binding cytoplasmic RNA interacting protein, component of the ApoB mRNA editosome involved in apolipoprotein B (APOB) mRNA editing, may bind RNA, may play a role in the regulation of mRNA splicing or transport from the nucleus. (PhosphoSite®, Cell Signaling Technology (Danvers, Mass.), Human PSD™, Biobase Corporation, (Beverly, Mass.)).

JJAZF1, phosphorylated at K341, is among the proteins listed in this patent. This protein has potential diagnostic and/or therapeutic implications based on the following findings. Translocation of the SUZ12 gene correlates with endometrial stromal tumors (Proc Natl Acad Sci USA 98: 6348-53. (2001)). Increased expression of SUZ12 protein correlates with colonic neoplasms (Genes Dev 18: 1592-605 (2004)). Increased expression of SUZ12 protein correlates with colonic neoplasms (Gene Develop 18: 1592-605 (2004)). Translocation of the SUZ12 gene correlates with endometrial stromal tumors (PNAS 98: 6348-53. (2001)). Translocation of the SUZ12 gene correlates with endometrial stromal tumors (Proc Natl Acad Sci USA 98: 6348-53. (2001)). Increased expression of SUZ12 protein correlates with colonic neoplasms (Genes Dev. 18: 1592-605 (2004)). Increased expression of SUZ12 protein correlates with colonic neoplasms (Genes Dev 18: 1592-605 (2004)). (PhosphoSite®, Cell Signaling Technology (Danvers, Mass.), Human PSD™, Biobase Corporation, (Beverly, Mass.)).

RIZ, phosphorylated at K1431 and K1425 is among the proteins listed in this patent. This protein has potential diagnostic and/or therapeutic implications based on the following findings. Decreased transcription coactivator activity of PRDM2 correlates with diffuse large-cell lymphoma (Gene Develop 15: 2250-62. (2001)). Decreased expression of PRDM2 mRNA may correlate with lung neoplasms (Cancer Res 58: 4238-44 (1998)). Gene instability of PRDM2 correlates with non-familial form of neoplasms (Cancer Res 60: 4701-4. (2000)). Decreased expression of PRDM2 mRNA may correlate with colorectal neoplasms (PNAS 97: 2662-7. (2000)). Loss of heterozygosity at the PRDM2 gene correlates with hepatocellular carcinoma (Br J Cancer 84: 743-7. (2001)). Hypermethylation of the PRDM2 promoter correlates with carcinoma tumors associated with stomach neoplasms (Int J Cancer 110: 212-8 (2004)). Gene instability of PRDM2 correlates with colorectal neoplasms (Proc Natl Acad Sci USA 97: 2662-7. (2000)). Decreased expression of PRDM2 mRNA may correlate with neuroblastoma (Cancer Res 58: 4238-44 (1998)). Decreased expression of PRDM2 mRNA correlates with Sezary syndrome associated with skin neoplasms (Cancer Res 64: 5578-86 (2004)). Missense mutation in the SET domain of PRDM2 correlates with diffuse large-cell lymphoma (Genes Dev 15: 2250-62. (2001)). Gene instability of PRDM2 correlates with colorectal neoplasms (Proc Natl Acad Sci USA 97: 2662-7. (2000)). Decreased expression of PRDM2 mRNA correlates with hepatocellular carcinoma (Int J Cancer 83: 541-6 (1999)). Frameshift mutation in the PRDM2 gene may correlate with colorectal neoplasms (Proc Natl Acad Sci USA 97: 2662-7. (2000)). Decreased expression of PRDM2 mRNA correlates with hepatocellular carcinoma associated with liver neoplasms (Int J Cancer 83: 541-6 (1999)). Frameshift mutation in the PRDM2 gene correlates with carcinoma tumors associated with endometrial neoplasms (Cancer Res 60: 4701-4. (2000)). Polymorphism in the PRDM2 gene correlates with hepatocellular carcinoma (Br J Cancer 84: 743-7. (2001)). Decreased transcription coactivator activity of PRDM2 correlates with diffuse large-cell lymphoma (GenesDev 15: 2250-62. (2001)). Decreased expression of PRDM2 mRNA correlates with abnormal G2/M transition of mitotic cell cycle associated with breast neoplasms (Cancer Res 58: 4238-44 (1998)). Frameshift mutation in the PRDM2 gene may correlate with colorectal neoplasms (PNAS 97: 2662-7. (2000)). Increased expression of PRDM2 mRNA may prevent increased cell proliferation associated with colorectal neoplasms (Cancer Res 61: 1796-8. (2001)). Deletion mutation in the PRDM2 gene correlates with colorectal neoplasms (PNAS 97: 2662-7. (2000)). Decreased transcription coactivator activity of PRDM2 correlates with diffuse large-cell lymphoma (Genes Dev 15: 2250-62. (2001)). Deletion mutation in the PRDM2 gene correlates with colorectal neoplasms (Proc Natl Acad Sci USA 97: 2662-7. (2000)). Decreased expression of PRDM2 mRNA may correlate with colorectal neoplasms (Proc Natl Acad Sci USA 97: 2662-7. (2000)). Frameshift mutation in the PRDM2 gene may correlate with colorectal neoplasms (Proc Natl Acad Sci USA 97: 2662-7. (2000)). Deletion mutation in the PRDM2 gene correlates with colorectal neoplasms (Proc Natl Acad Sci USA 97: 2662-7. (2000)). Alternative form of PRDM2 protein correlates with breast neoplasms (Cancer Res 58: 4238-44 (1998)). Decreased transcription coactivator activity of PRDM2 correlates with diffuse large-cell lymphoma (Genes Dev. 15: 2250-62. (2001)). Missense mutation in the SET domain of PRDM2 correlates with diffuse large-cell lymphoma (Genes Dev. 15: 2250-62. (2001)). Frameshift mutation in the PRDM2 gene correlates with carcinoma tumors associated with stomach neoplasms (Cancer Res 60: 4701-4. (2000)). Missense mutation in the SET domain of PRDM2 correlates with diffuse large-cell lymphoma (Genes Dev 15: 2250-62. (2001)). Frameshift mutation in the PRDM2 gene correlates with carcinoma tumors associated with colorectal neoplasms (Cancer Res 60: 4701-4. (2000)). Decreased expression of PRDM2 mRNA may correlate with colorectal neoplasms (Proc Natl Acad Sci USA 97: 2662-7. (2000)). Missense mutation in the SET domain of PRDM2 correlates with diffuse large-cell lymphoma (Gene Develop 15: 2250-62. (2001)). Polymorphism in the PRDM2 gene correlates with increased incidence of hepatocellular carcinoma associated with liver neoplasms (Br J Cancer 84: 743-7. (2001)). Gene instability of PRDM2 correlates with colorectal neoplasms (PNAS 97: 2662-7. (2000)). Decreased expression of PRDM2 mRNA correlates with Sezary syndrome (Cancer Res 64: 5578-86 (2004)). (PhosphoSite®, Cell Signaling Technology (Danvers, Mass.), Human PSD™, Biobase Corporation, (Beverly, Mass.)).

eEF1A-1, phosphorylated at K179, is among the proteins listed in this patent. eEF1A-1, CC chemokine receptor 5, a G protein-coupled receptor that binds chemokines and is a coreceptor for HIV-1 glycoprotein 120, may modulate immune and inflammatory responses, inhibition may be therapeutic for HIV infections and multiple sclerosis. This protein has potential diagnostic and/or therapeutic implications based on the following findings. Induced inhibition of the coreceptor activity of CCR5 may prevent HIV infections (J Virol 74: 9328-32. (2000)). Polymorphism in the CCR5 gene correlates with increased incidence of death associated with breast neoplasms (J Exp Med 198: 1381-9. (2003)). Deletion mutation in the CCR5 gene correlates with late onset form of HIV infections (Mol Med 6: 28-36 (2000)). Increased expression of CCR5 in T-lymphocytes correlates with more severe form of HIV infections (J Infect Dis 181: 927-32 (2000)). Induced inhibition of the viral receptor activity of CCR5 may prevent abnormal initiation of viral infection associated with HIV infections (Cell 86: 367-77 (1996)). Induced inhibition of the coreceptor activity of CCR5 may prevent HIV infections (J Virol 73: 3443-8. (1999)). Polymorphism in the CCR5 promoter correlates with increased occurrence of acquired immunodeficiency syndrome associated with HIV infections (Science 282: 1907-11 (1998)). Deletion mutation in the CCR5 gene correlates with decreased occurrence of disease susceptibility associated with asthma (Lancet 354: 1264-5 (1999)). Increased expression of CCR5 in leukocytes correlates with pulmonary tuberculosis associated with AIDS-related opportunistic infections (J Infect Dis 183: 1801-4. (2001)). Decreased expression of CCR5 in T-lymphocytes correlates with abnormal T-lymphocytes migration associated with chronic hepatitis C (J Infect Dis 185: 1803-7. (2002)). Increased expression of CCR5 mRNA correlates with inflammation (J Clin Invest 101: 746-54. (1998)). Decreased expression of CCR5 in leukocytes correlates with type I diabetes mellitus (Diabetes 51: 2474-80. (2002)). Monoclonal antibody to CCR5 may prevent abnormal initiation of viral infection associated with HIV infections (PNAS 97: 3388-93 (2000)). Increased expression of CCR5 in T-lymphocytes correlates with schistosomiasis mansoni (Infect Immun 71: 6668-71. (2003)). Polymorphism in the CCR5 gene correlates with increased occurrence of disease susceptibility associated with diabetic nephropathies (Diabetes 54: 3331-5 (2005)). Increased expression of CCR5 in T-lymphocytes correlates with advanced stage or high grade form of HIV infections (J Immunol 163: 4597-603 (1999)). Viral exploitation of the coreceptor activity of CCR5 may cause HIV infections (J Virol 79: 1686-700 (2005)). Increased viral receptor activity of CCR5 correlates with advanced stage or high grade form of acquired immunodeficiency syndrome (J Virol 73: 9741-55. (1999)). Increased expression of CCR5 in dendritic cells correlates with optic neuritis associated with multiple sclerosis (Clin Exp Immunol 127: 519-26. (2002)). Abnormal expression of CCR5 protein correlates with Graves’ disease (Clin Exp Immunol 127: 479-85. (2002)). Monoclonal antibody to CCR5 may prevent abnormal initiation of viral infection associated with HIV infections (Proc Natl Acad Sci USA 97: 3388-93 (2000)). Increased expression of CCR5 in T-lymphocytes correlates with inflammation associated with chronic hepatitis C (J Infect Dis 190: 989-97 (2004)). Increased expression of CCR5 in monocytes correlates with schistosomiasis mansoni (Infect Immun 71: 6668-71. (2003)). Polymorphism in the CCR5 gene correlates with diabetic angiopathies associated with type 1 diabetes mellitus (Cytokine 26: 114-21 (2004)). Decreased plasma membrane localization of CCR5 may prevent HIV infections (PNAS 94: 11567-72 (1997)). Viral exploitation of the coreceptor activity of CCR5 may cause defective initiation of viral infection associated with HIV infections (J Virol 71: 7478-87. (1997)). Increased expression of CCR5 in T-lymphocytes correlates with more severe form of HIV infections (Blood 96: 2649-54 (2000)). Increased expression of CCR5 protein correlates with kidney diseases (Kidney Int 56: 52-64 (1999)). Single nucleotide polymorphism in the CCR5 promoter correlates with diabetic nephropathies (Diabetes 51: 238-42. (2002)). Polymorphism in the CCR5 promoter correlates with increased occurrence of disease progression associated with acquired immunodeficiency syndrome (Science 282: 1907-11 (1998)). Polymorphism in the CCR5 gene correlates with decreased occurrence of AIDS-related lymphoma associated with acquired immunodeficiency syndrome (Blood 93: 1838-42. (1999)). Induced inhibition of the viral receptor activity of CCR5 may prevent abnormal initiation of viral infection associated with HIV infections (Nature 382: 722-5 (1996)). Polymorphism in the CCR5 promoter correlates with more severe form of HIV infections (J Infect Dis 183: 814-8. (2001)). Increased expression of CCR5 in B-lymphocytes correlates with inflammation associated with chronic hepatitis C (J Infect Dis 190: 989-97 (2004)). Induced inhibition of the coreceptor activity of CCR5 may prevent HIV infections (PNAS 98: 12718-23. (2001)). Decreased plasma membrane localization of CCR5 may prevent HIV infections (Proc Natl Acad Sci USA 94: 11567-72 (1997)). Decreased expression of CCR5 in T-lymphocytes may prevent HIV infections (PNAS 100: 183-8. (2003)). Absence of plasma membrane localization of CCR5 causes decreased initiation of viral infection associated with HIV infections (Cell 86: 367-77 (1996)). Abnormal expression of CCR5 in T-lymphocytes correlates with rheumatoid arthritis (Clin Exp Immunol 132: 371-8. (2003)). Viral exploitation of the chemokine receptor activity of CCR5 may cause increased initiation of viral infection associated with acquired immunodeficiency syndrome (Proc Natl Acad Sci USA 96: 7496-501 (1999)). Increased expression of CCR5 in T-lymphocytes correlates with rheumatoid arthritis (J Immunol 174: 1693-700 (2005)). Viral exploitation of the coreceptor activity of CCR5 causes increased initiation of viral infection associated with HIV infections (Cell 85: 1135-48 (1996)). Deletion mutation in the CCR5 gene correlates with abnormal immune response associated with HIV infections (Mol Med 6: 28-36 (2000)). Single nucleotide polymorphism in the CCR5 promoter correlates with increased incidence of diabetic nephropathies associated with type II diabetes mellitus (Diabetes 51: 238-42. (2002)). Deletion mutation in the CCR5 gene correlates with decreased occurrence of non-Hodgkin's lymphoma associated with acquired immunodeficiency syndrome (Blood 93: 1838-42. (1999)). Antibody to CCR5 may prevent increased initiation of viral infection associated with HIV infections (Proc Natl Acad Sci USA 97: 805-10 (2000)). Increased expression of CCR5 in T-lymphocytes may correlate with pulmonary tuberculosis associated with HIV infections (J Infect Dis 183: 1801-4. (2001)). Viral exploitation of the coreceptor activity of CCR5 correlates with acute form of HIV infections (Blood 98: 3169-71. (2001)). Increased expression of CCR5 in lymphocytes correlates with chronic hepatitis C (J Immunol 163: 6236-43 (1999)). Increased expression of CCR5 in fibroblasts correlates with rheumatoid arthritis (J Immunol 167: 5381-5. (2001)). Increased expression of CCR5 in T-lymphocytes may correlate with AIDS-related opportunistic infections associated with HIV infections (J Infect Dis 183: 1801-4. (2001)). Mutation in the CCR5 gene correlates with decreased occurrence of acquired immunodeficiency syndrome associated with HIV infections (Science 277: 959-65 (1997)). Decreased expression of CCR5 in T-lymphocytes may prevent HIV infections (Proc Natl Acad Sci USA 100: 183-8. (2003)). Increased expression of CCR5 protein correlates with inflammation associated with periodontitis (Cytokine 20: 70-7. (2002)). Polymorphism in the CCR5 promoter correlates with more severe form of HIV infections (J Infect Dis 184: 89-92. (2001)). Loss of function mutation in the CCR5 gene causes decreased initiation of viral infection associated with HIV infections (Mol Med 3: 23-36. (1997)). Decreased chemokine receptor activity of CCR5 correlates with decreased occurrence of recurrence associated with multiple sclerosis (J Neuroimmunol 102: 98-106. (2000)). Decreased expression of CCR5 in T-lymphocytes correlates with Crohn disease (Clin Exp Immunol 132: 332-8. (2003)). Viral exploitation of the chemokine receptor activity of CCR5 may cause increased initiation of viral infection associated with acquired immunodeficiency syndrome (PNAS 96: 7496-501 (1999)). Viral exploitation of the CCR5 protein causes increased entry of virus into host cell associated with HIV infections (J Neuroimmunol 110: 230-9 (2000)). Deletion mutation in the CCR5 gene correlates with decreased occurrence of AIDS-related lymphoma associated with acquired immunodeficiency syndrome (Blood 93: 1838-42. (1999)). Increased expression of CCR5 in lymphocytes correlates with increased T-helper 1 type immune response associated with Behcet Syndrome (Clin Exp Immunol 139: 371-8 (2005)). Antibody to CCR5 may prevent increased initiation of viral infection associated with HIV infections (PNAS 97: 805-10 (2000)). Increased presence of CCR5 antibody may prevent HIV infections (Clin Exp Immunol 129: 493-501. (2002)). Viral exploitation of the coreceptor activity of CCR5 correlates with AIDS dementia complex (Virology 279: 509-26. (2001)). Antibody to CCR5 may prevent increased initiation of viral infection associated with HIV infections (Proc Natl Acad Sci USA 97: 805-10 (2000)). Viral exploitation of the coreceptor activity of CCR5 causes increased initiation of viral infection associated with HIV infections (J Exp Med 185: 621-8. (1997)). Induced inhibition of the chemokine receptor activity of CCR5 may prevent recurrence associated with multiple sclerosis (J Neuroimmunol 102: 98-106. (2000)). Increased expression of CCR5 in lymphocytes correlates with autoimmune diseases associated with thyroid diseases (J Clin Endocrinol Metab 86: 5008-16. (2001)). Viral exploitation of the chemokine receptor activity of CCR5 may cause increased initiation of viral infection associated with acquired immunodeficiency syndrome (Proc Natl Acad Sci USA 96: 7496-501 (1999)). Loss of function mutation in the CCR5 gene correlates with decreased severity of disease progression associated with HIV infections (Mol Med 3: 23-36. (1997)). Absence of the viral receptor activity of CCR5 causes decreased initiation of viral infection associated with HIV infections (Nature 382: 722-5 (1996)). Polymorphism in the CCR5 promoter correlates with more severe form of HIV infections (J Virol 73: 10264-71. (1999)). Increased expression of CCR5 in leukocytes correlates with AIDS-related opportunistic infections associated with HIV infections (J Infect Dis 183: 1801-4. (2001)). Deletion mutation in the CCR5 gene correlates with decreased occurrence of recurrence associated with multiple sclerosis (J Neuroimmunol 102: 98-106. (2000)). Induced inhibition of the coreceptor activity of CCR5 may prevent HIV infections (Proc Natl Acad Sci USA 98: 12718-23. (2001)). Absence of plasma membrane localization of CCR5 causes decreased initiation of viral infection associated with HIV infections (Nature 382: 722-5 (1996)). Increased expression of CCR5 in T-lymphocytes may correlate with pulmonary tuberculosis associated with AIDS-related opportunistic infections (J Infect Dis 183: 1801-4. (2001)). Deletion mutation in the CCR5 gene may prevent disease progression associated with acquired immunodeficiency syndrome (Science 273: 1856-62 (1996)). Deletion mutation in the CCR5 gene causes decreased initiation of viral infection associated with HIV infections (Nature 382: 722-5 (1996)). Viral exploitation of the chemokine receptor activity of CCR5 may cause increased induction by virus of cell-cell fusion in host associated with HIV infections (J Virol 71: 8405-15. (1997)). Polymorphism in the CCR5 gene correlates with decreased (delayed) early viral mRNA transcription associated with HIV seropositivity (J Virol 76: 662-72. (2002)). Absence of the viral receptor activity of CCR5 causes decreased initiation of viral infection associated with HIV infections (Cell 86: 367-77 (1996)). Increased expression of CCR5 in leukocytes correlates with pulmonary tuberculosis associated with HIV infections (J Infect Dis 183: 1801-4. (2001)). Deletion mutation in the CCR5 gene may prevent HIV infections (Science 273: 1856-62 (1996)). Increased expression of CCR5 in monocytes correlates with more severe form of HIV infections (J Exp Med 187: 439-44. (1998)). Increased expression of CCR5 in B-lymphocytes correlates with relapsing-remitting multiple sclerosis (J Neuroimmunol 122: 125-31. (2002)). Increased expression of CCR5 mRNA correlates with periapical granuloma (Cytokine 16: 62-6. (2001)). Deletion mutation in the CCR5 gene causes decreased initiation of viral infection associated with HIV infections (Cell 86: 367-77 (1996)). Increased expression of CCR5 in B-lymphocytes correlates with Hodgkin's disease (Blood 97: 1543-8. (2001)). Abnormal expression of CCR5 in NK cells may correlate with increased severity of leukemia associated with lymphoproliferative disorders (Leukemia 19: 1169-74 (2005)). Increased expression of CCR5 in T-lymphocytes may cause increased T-helper 1 type immune response associated with relapsing-remitting multiple sclerosis (J Neuroimmunol 114: 207-12. (2001)). Viral explbitation of the CCR5 protein may cause increased induction by virus of cell-cell fusion in host associated with HIV infections (Blood 103: 1211-7 (2004)). Monoclonal antibody to CCR5 may prevent abnormal initiation of viral infection associated with HIV infections (Proc Natl Acad Sci USA 97: 3388-93 (2000)). Induced inhibition of the coreceptor activity of CCR5 may prevent HIV infections (Proc Natl Acad Sci USA 98: 12718-23. (2001)). Polymorphism in the CCR5 promoter correlates with decreased occurrence of acquired immunodeficiency syndrome associated with HIV infections (Lancet 352: 866-70. (1998)). Increased expression of CCR5 in T-lymphocytes correlates with Hodgkin's disease (Blood 97: 1543-8. (2001)). Polymorphism in the CCR5 gene correlates with increased initiation of viral infection associated with HIV infections (J Infect Dis 183: 1574-85. (2001)). Decreased expression of CCR5 protein correlates with chronic myeloid leukemia (J Immunol 162: 6191-9 (1999)). Decreased plasma membrane localization of CCR5 may prevent HIV infections (Proc Natl Acad Sci USA 94: 11567-72 (1997)). Decreased expression of CCR5 in T-lymphocytes may prevent HIV infections (Proc Natl Acad Sci USA 100: 183-8. (2003)). Viral exploitation of the chemokine receptor activity of CCR5 may cause increased induction by virus of cell-cell fusion in host associated with acquired immunodeficiency syndrome (J Virol 71: 8405-15. (1997)). Increased expression of CCR5 in NK cells correlates with inflammation associated with chronic hepatitis C (J Infect Dis 190: 989-97 (2004)). (PhosphoSite®, Cell Signaling Technology (Danvers, Mass.), Human PSD™, Biobase Corporation, (Beverly, Mass.)).

EEF1G, phosphorylated at K147 and K212, is among the proteins listed in this patent. This protein has potential diagnostic and/or therapeutic implications based on the following findings. Increased expression of EEF1G in epithelium/epithelial cells correlates with increased occurrence of advanced stage or high grade form of colorectal neoplasms (Cancer 82: 816-21. (1998)). Missense mutation in the EEFIG gene may correlate with carcinoma tumors associated with colorectal neoplasms (Mol Carcinog 22: 9-15. (1998)). Increased expression of EEF1G mRNA correlates with carcinoma tumors associated with stomach neoplasms (Cancer 75: 1446-9. (1995)). Increased expression of EEF1G mRNA correlates with adenoma tumors associated with colorectal neoplasms (Mol Carcinog 7: 18-20. (1993)). (PhosphoSite®, Cell Signaling Technology (Danvers, Mass.), Human PSD™, Biobase Corporation, (Beverly, Mass.)).

eEF-2, phosphorylated at K426 and K235 is among the proteins listed in this patent. This protein has potential diagnostic and/or therapeutic implications based on the following findings. Mutation in the EEF2 gene may correlate with squamous cell carcinoma (Cancer Res 58: 5144-50. (1998)). Increased expression of EEF2 protein correlates with ovarian neoplasms (Int J Cancer 73: 678-83 (1997)). Induced inhibition of the translation elongation factor activity of EEF2 may prevent increased anti-apoptosis associated with breast neoplasms (Biochemistry Usa 37: 16934-42. (1998)). Induced inhibition of the translation elongation factor activity of EEF2 may prevent increased cell proliferation associated with squamous cell carcinoma (Biochem J: 737-41. (1997)). Induced inhibition of the translation elongation factor activity of EEF2 may prevent increased anti-apoptosis associated with breast neoplasms (Biochemistry 37: 16934-42. (1998)). (PhosphoSite®, Cell Signaling Technology (Danvers, Mass.), Human PSD™, Biobase Corporation, (Beverly, Mass.)).

eIF3-eta, phosphorylated at K729, is among the proteins listed in this patent. eIF3-eta, Eukaryotic translation initiation factor 3 subunit 9, a subunit of the EIF3 complex that plays a role in protein synthesis initiation. (PhosphoSite®, Cell Signaling Technology (Danvers, Mass.), Human PSD™, Biobase Corporation, (Beverly, Mass.)).

eIF4B, phosphorylated at K586, is among the proteins listed in this patent. eIF4B, Eukaryotic translation initiation factor 4B, an RNA binding protein, may be involved in both mRNA cap-dependent and cap-independent translation initiation, cleavage by CASP3 during apoptosis results in a decrease of overall protein synthesis. (PhosphoSite®, Cell Signaling Technology (Danvers, Mass.), Human PSD™, Biobase Corporation, (Beverly, Mass.)).

eIF5A, phosphorylated at K67, is among the proteins listed in this patent. eIF5A, Eukaryotic translation initiation factor 5A, a translation initiation factor that exports HIV1 viral mRNA from the nucleus, plays a role in cell proliferation and cytokine signaling, modulates p53 (TP53) expression and inhibits apoptosis. (PhosphoSite®, Cell Signaling Technology (Danvers, Mass.), Human PSD™, Biobase Corporation, (Beverly, Mass.)).

RPL10, phosphorylated at K208, is among the proteins listed in this patent. This protein has potential diagnostic and/or therapeutic implications based on the following findings. Increased expression of RPL10 mRNA may correlate with decreased response to hormone stimulus associated with prostatic neoplasms (Carcinogenesis 23: 967-75. (2002)). Increased expression of RPL10 mRNA may correlate with increased severity of disease progression associated with prostatic neoplasms (Carcinogenesis 23: 967-75. (2002)). Abnormal expression of RPL10 mRNA may correlate with Wilm's tumor tumors associated with nephroblastomas (Nucleic Acids Res 19: 5763-9 (1991)). Abnormal expression of RPL10 mRNA may correlate with invasive form of nephroblastomas (Nucleic Acids Res 19: 5763-9 (1991)). (PhosphoSite®, Cell Signaling Technology (Danvers, Mass.), Human PSD™, Biobase Corporation, (Beverly, Mass.)).

RPL11, phosphorylated at K52, is among the proteins listed in this patent. RPL11, Ribosomal protein L11, a component of the 60S ribosomal subunit that is involved in protein biosynthesis, cell cycle arrest, and transcription, plays a role in TP53 stabilization and activation via ihibition of the ubiquitin ligase activity of MDM2. (PhosphoSite®, Cell Signaling Technology (Danvers, Mass.), Human PSD™, Biobase Corporation, (Beverly, Mass.)).

RPL5, phosphorylated at K220, is among the proteins listed in this patent. This protein has potential diagnostic and/or therapeutic implications based on the following findings. Increased expression of RPL5 mRNA correlates with adenocarcinoma tumors associated with colonic neoplasms (MCB 11: 3842-9 (1991)). Increased expression of RPL5 mRNA correlates with adenocarcinoma tumors associated with colonic neoplasms (Mol Cell Biol 11: 3842-9 (1991)). Increased expression of RPL5 mRNA correlates with adenocarcinoma tumors associated with colonic neoplasms (Mol Cell Biol. 11: 3842-9 (1991)). Increased expression of RPL5 mRNA correlates with adenocarcinoma tumors associated with colonic neoplasms (Mol. Cell Biol 11: 3842-9 (1991)). Increased expression of RPL5 mRNA correlates with adenomatous polyps (MCB 11: 3842-9 (1991)). Increased expression of RPL5 mRNA correlates with adenomatous polyps (Mol Cell Biol. 11: 3842-9 (1991)). Increased expression of RPL5 mRNA correlates with adenomatous polyps (Mol. Cell. Biol. 11: 3842-9 (1991)). Increased expression of RPL5 mRNA correlates with adenomatous polyps (Mol. Cell Biol 11: 3842-9 (1991)). Increased expression of RPL5 mRNA correlates with adenomatous polyps (Mol Cell Biol 11: 3842-9 (1991)). Increased presence of RPL5 autoimmune antibody correlates with increased severity of nephritis associated with systemic lupus erythematosus (Clin Exp Immunol 95: 385-9. (1994)). Increased expression of RPL5 mRNA correlates with disease progression associated with hepatocellular carcinoma (Anticancer Res 20: 2489-94. (2000)). Increased expression of RPL5 mRNA correlates with adenocarcinoma tumors associated with colonic neoplasms (Mol. Cell. Biol. 11: 3842-9 (1991)). Increased expression of RPL5 mRNA may correlate with prostatic neoplasms (Int J Cancer 78: 27-32. (1998)). Increased expression of RPL5 mRNA may correlate with drug-resistant form of colonic neoplasms (Eur J Cancer 34: 731-6 (1998)). Increased expression of RPL5 mRNA correlates with astrocytoma (Cancer Res 60: 6868-74. (2000)). (PhosphoSite®, Cell Signaling Technology (Danvers, Mass.), Human PSD™, Biobase Corporation, (Beverly, Mass.)).

RPLP2, phosphorylated at K21, is among the proteins listed in this patent. This protein has potential diagnostic and/or therapeutic implications based on the following findings. Decreased expression of RPLP2 protein may cause decreased translation associated with breast neoplasms (Cancer Res 62: 1036-44. (2002)). Increased presence of RPLP2 autoimmune antibody correlates with allergic bronchopulmonary aspergillosis (J Exp Med 189: 1507-12 (1999)). Decreased expression of RPLP2 protein may cause decreased cell proliferation associated with breast neoplasms (Cancer Res 62: 1036-44. (2002)). Increased presence of RPLP2 autoimmune antibody correlates with systemic lupus erythematosus (J Clin Invest 94: 345-52 (1994)). RPLP2 epitope causes increased cell-mediated immune response associated with allergic bronchopulmonary aspergillosis (J Exp Med 189: 1507-12 (1999)). RPLP2 epitope causes increased humoral immune response associated with allergic bronchopulmonary aspergillosis (J Exp Med 189: 1507-12 (1999)). Increased expression of RPLP2 mRNA correlates with benign form of breast neoplasms (Br J Cancer 65: 65-71. (1992)). Increased expression of RPLP2 mRNA correlates with hepatocellular carcinoma (Anticancer Res 20: 2489-94. (2000)). Increased expression of RPLP2 mRNA may correlate with pancreatic neoplasms (Biochem Biophys Res Commun 293: 391-5. (2002)). Increased expression of RPLP2 mRNA correlates with hepatocellular carcinoma associated with liver neoplasms (Anticancer Res 20: 2489-94. (2000)). Increased presence of RPLP2 autoimmune antibody may correlate with increased proliferation of active T-cells associated with systemic lupus erythematosus (J Clin Invest 94: 345-52 (1994)). Increased expression of RPLP2 mRNA correlates with fibroadenoma associated with breast neoplasms (Br J Cancer 61: 83-8. (1990)). (PhosphoSite®, Cell Signaling Technology (Danvers, Mass.), Human PSD™, Biobase Corporation, (Beverly, Mass.)).

TUFM, phosphorylated at K82, is among the proteins listed in this patent. TUFM, Tu translation elongation factor (mitochondrial), a putative translation elongation factor, may be involved in protein biosynthesis, upregulated in some tumors. (PhosphoSite®, Cell Signaling Technology (Danvers, Mass.), Human PSD™, Biobase Corporation, (Beverly, Mass.)).

SAE2, phosphorylated at K253, is among the proteins listed in this patent. SAE2, SUMO-1 activating enzyme subunit 2, involved in the activation of the ubiquitin-like protein SUMO-1 (UBL1), which leads to alteration of the subcellular distribution and stability of selected proteins. (PhosphoSite®, Cell Signaling Technology (Danvers, Mass.), Human PSD™, Biobase Corporation, (Beverly, Mass.)).

SKP1A, phosphorylated at K163, is among the proteins listed in this patent. This protein has potential diagnostic and/or therapeutic implications based on the following findings. Abnormal expression of SKP1A protein may cause abnormal regulation of progression through cell cycle associated with neoplasms (Proc Natl Acad Sci USA 95: 11324-9. (1998)). Abnormal expression of SKP1A protein may cause abnormal regulation of progression through cell cycle associated with neoplasms (Proc Natl Acad Sci USA 95: 11324-9. (1998)). Abnormal expression of SKP1A protein may cause abnormal regulation of progression through cell cycle associated with neoplasms (PNAS 95: 11324-9. (1998)). (PhosphoSite®, Cell Signaling Technology (Danvers, Mass.), Human PSD™, Biobase Corporation, (Beverly, Mass.)).

UBE2N, phosphorylated at K92, is among the proteins listed in this patent. UBE2N, Ubiquitin-conjugating enzyme E2N (yeast UBC13 homolog), forms heterodimers with ubiquitin-conjugating enzyme E2 variants 1 or 2 (UBE2V1, UBE2V2), and catalyzes formation of unique lysine 63-linked polyubiquitin chains involved in IkappaB kinase activation. (PhosphoSite®, Cell Signaling Technology (Danvers, Mass.), Human PSD™, Biobase Corporation, (Beverly, Mass.)).

ZNF198, phosphorylated at K1280, is among the proteins listed in this patent. This protein has potential diagnostic and/or therapeutic implications based on the following findings. Translocation of the ZMYM2 gene correlates with myeloproliferative disorders (Genomics 55: 118-21. (1999)). Translocation of the ZMYM2 gene may cause abnormal signal transduction associated with myeloproliferative disorders (Hum Mol Genet 7: 637-42 (1998)). Translocation of the ZMYM2 gene correlates with myeloproliferative disorders (Proc Natl Acad Sci USA 95: 5712-7 (1998)). Translocation of the ZMYM2 gene correlates with myeloproliferative disorders (Proc Natl Acad Sci USA 95: 5712-7 (1998)). Translocation of the ZMYM2 gene correlates with myeloproliferative disorders (PNAS 95: 5712-7 (1998)). Translocation of the ZMYM2 gene may cause abnormal signal transduction associated with myeloproliferative disorders (Blood 92: 1735-42 (1998)). Translocation of the ZMYM2 gene correlates with myeloproliferative disorders (Nat Genet 18: 84-7 (1998)). (PhosphoSite®, Cell Signaling Technology (Danvers, Mass.), Human PSD™, Biobase Corporation, (Beverly, Mass.)).

GLG1, phosphorylated at K747, is among the proteins listed in this patent. GLG1, Golgi apparatus protein 1, a cysteine-rich membrane sialoglycoprotein, may be a receptor for fibroblast growth factor (FGF), may be involved in cell adhesion, may play a role in the malignant progression in astrocytomas. (PhosphoSite®, Cell Signaling Technology (Danvers, Mass.), Human PSD™, Biobase Corporation, (Beverly, Mass.)).

SNX1, phosphorylated at K237, is among the proteins listed in this patent. SNX1, Sorting nexin 1, binds and mediates endosomal trafficking of various cell surface tyrosine kinase receptors and GPCRs, plays a role in lysosomal sorting and degradation of epidermal growth factor receptor (EGFR) and PAR-1 (F2R). (PhosphoSite®, Cell Signaling Technology (Danvers, Mass.), Human PSD™, Biobase Corporation, (Beverly, Mass.)).

The invention also provides peptides comprising a novel acetylation site of the invention. In one particular embodiment, the peptides comprise any one of the an amino acid sequences as set forth in column E of Table 1 and FIG. 2, which are trypsin-digested peptide fragments of the parent proteins. Alternatively, a parent signaling protein listed in Table 1 may be digested with another protease, and the sequence of a peptide fragment comprising a acetylation site can be obtained in a similar way. Suitable proteases include, but are not limited to, serine proteases (e.g. hepsin), metallo proteases (e.g. PUMP1), chymotrypsin, cathepsin, pepsin, thermolysin, carboxypeptidases, etc.

The invention also provides proteins and peptides that are mutated to eliminate a novel acetylation site of the invention. Such proteins and peptides are particular useful as research tools to understand complex signaling transduction pathways of cancer cells, for example, to identify new upstream acetylase(s) or deacetylase(s) or other proteins that regulates the activity of a signaling protein; to identify downstream effector molecules that interact with a signaling protein, etc.

Various methods that are well known in the art can be used to eliminate a acetylation site. For example, the acetylatable lysine may be mutated into a non-acetylatable residue, such as glutamine. An “acetylatable” amino acid refers to an amino acid that is capable of being modified by addition of a and acetyl group (any includes both acetylated form and unacetylated form). Alternatively, the lysine may be deleted. Residues other than the lysine may also be modified (e.g., delete or mutated) if such modification inhibits the acetylation of the lysine residue. For example, residues flanking the lysine may be deleted or mutated, so that an acetylase can not recognize/acetylate the mutated protein or the peptide. Standard mutagenesis and molecular cloning techniques can be used to create amino acid substitutions or deletions.

### 2. Modulators of the Acetylation Sites

In another aspect, the invention provides a modulator that modulates lysine acetylation at a novel acetylation site of the invention, including small molecules, peptides comprising a novel acetylation site, and binding molecules that specifically bind at a novel acetylation site, including but not limited to antibodies or antigen-binding fragments thereof.

Modulators of an acetylation site include any molecules that directly or indirectly counteract, reduce, antagonize or inhibit lysine acetylation of the site. The modulators may compete or block the binding of the acetylation site to its upstream acetylase(s) or deacetylase(s), or to its downstream signaling transduction molecule(s).

The modulators may directly interact with an acetylation site. The modulator may also be a molecule that does not directly interact with an acetylation site. For example, the modulators can be dominant negative mutants, i.e., proteins and peptides that are mutated to eliminate the acetylation site. Such mutated proteins or peptides could retain the binding ability to a downstream signaling molecule but lose the ability to trigger downstream signaling transduction of the wild type parent signaling protein.

The modulators include small molecules that modulate the lysine acetylation at a novel acetylation site of the invention. Chemical agents, referred to in the art as “small molecule” compounds are typically organic, non-peptide molecules, having a molecular weight less than 10,000, less than 5,000, less than 1,000, or less than 500 daltons. This class of modulators includes chemically synthesized molecules, for instance, compounds from combinatorial chemical libraries. Synthetic compounds may be rationally designed or identified based on known or inferred properties of an acetylation site of the invention or may be identified by screening compound libraries. Alternative appropriate modulators of this class are natural products, particularly secondary metabolites from organisms such as plants or fungi, which can also be identified by screening compound libraries. Methods for generating and obtaining compounds are well known in the art (Schreiber S L, Science 151: 1964-1969(2000); Radmann J. and Gunther J., Science 151: 1947-1948 (2000)).

The modulators also include peptidomimetics, small protein-like chains designed to mimic peptides. Peptidomimetics may be analogues of a peptide comprising a acetylation site of the invention. Peptidomimetics may also be analogues of a modified peptide that are mutated to eliminate an acetylation site of the invention. Peptidomimetics (both peptide and non-peptidyl analogues) may have improved properties (e.g., decreased proteolysis, increased retention or increased bioavailability). Peptidomimetics generally have improved oral availability, which makes them especially suited to treatment of disorders in a human or animal.

In certain embodiments, the modulators are peptides comprising a novel acetylation site of the invention. In certain embodiments, the modulators are antibodies or antigen-binding fragments thereof that specifically bind at a novel acetylation site of the invention.

### 3. Heavy-Isotope Labeled Peptides (AQUA Peptides).

In another aspect, the invention provides peptides comprising a novel acetylation site of the invention. In a particular embodiment, the invention Provides Heavy-Isotype Labeled Peptides (AQUA peptides) comprising a novel acetylation site. Such peptides are useful to generate acetylation site-specific antibodies for a novel acetylation site. Such peptides are also useful as potential diagnostic tools for screening different types of cancer including carcinoma, or as potential therapeutic agents for treating cancer including carcinoma.

The peptides may be of any length, typically six to fifteen amino acids. The novel lysine acetylation site can occur at any position in the peptide; if the peptide will be used as an immnogen, it preferably is from seven to twenty amino acids in length. In some embodiments, the peptide is labeled with a detectable marker.

“Heavy-isotope labeled peptide” (used interchangeably with AQUA peptide) refers to a peptide comprising at least one heavy-isotope label, as described in WO/03016861, “Absolute Quantification of Proteins and Modified Forms Thereof by Multistage Mass Spectrometry” (Gygi et al.) (the teachings of which are hereby incorporated herein by reference, in their entirety). The amino acid sequence of an AQUA peptide is identical to the sequence of a proteolytic fragment of the parent protein in which the novel acetylation site occurs. AQUA peptides of the invention are highly useful for detecting, quantitating or modulating an acetylation site of the invention (both in acetylated and unacetylated forms) in a biological sample.

A peptide of the invention, including an AQUA peptides comprises any novel acetylation site. Preferably, the peptide or AQUA peptide comprises a novel acetylation site of a protein in Table 1 that is a chormatin or DNA binding/repair/replication protein, enzyme protein, RNA binding protein, transcriptional regulator, translational regulator, ubiquitan conjugating system, cytoskeletal protein, adaptor/scaffold protein or receptor/channel/transporter/cell surface protein.

Particularly preferred peptides and AQUA peptides are these comprising a novel lysine acetylation site (shown as a lower case “k” in a sequence listed in Table 1) selected from the group consisting of SEQ ID NOs: 28 (MYST3); 51 (HEMH); 55 (PDA6); 56 (PDA6); 57 (PPIL2); 58 (RSAFD1); 96 (KHSRP); 104 (NSAP1); 143 (eEF1A-1); 145 (EEF1G); 147 (eEF-2); 149 (eIF3-eta); 152 (eIF5A); 155 (RPL10A); 157 (RPL11); 159 (RPL13A); 161 (RPL18a); 162 (RPL24); 164 (RPL5); 166 (RPLP2); 169(RPS5); 172(TUFM); 176 (SAE2); 177 (SKP1A); 178 (UBE2N); 179 (UBE2S); 180 (UCHL5); 41 (gelsolin); 7(NMD3); 80 (SLC25A5); 67 (OTUB1); 225 (DOCK7); 286 (LEREPO4); 316 (NSUN2); and 388 (NSFL1C).

In some embodiments, the peptide or AQUA peptide comprises the amino acid sequence shown in any one of the above listed SEQ ID NOs. In some embodiments, the peptide or AQUA peptide consists of the amino acid sequence in said SEQ ID NOs. In some embodiments, the peptide or AQUA peptide comprises a fragment of the amino acid sequence in said SEQ ID NOs., wherein the fragment is six to twenty amino acid long and includes the acetylatable lysine. In some embodiments, the peptide or AQUA peptide consists of a fragment of the amino acid sequence in said SEQ ID NOs., wherein the fragment is six to twenty amino acid long and includes the acetylatable lysine.

In certain embodiments, the peptide or AQUA peptide comprises any one of the SEQ ID NOs listed in column H, which are trypsin-digested peptide fragments of the parent proteins.

It is understood that parent protein listed in Table 1 may be digested with any suitable protease (e.g., serine proteases (e.g. trypsin, hepsin), metallo proteases (e.g. PUMP1), chymotrypsin, cathepsin, pepsin, thermolysin, carboxypeptidases, etc), and the resulting peptide sequence comprising a acetylated site of the invention may differ from that of trypsin-digested fragments (as set forth in Column E), depending the cleavage site of a particular enzyme. An AQUA peptide for a particular a parent protein sequence should be chosen based on the amino acid sequence of the parent protein and the particular protease for digestion; that is, the AQUA peptide should match the amino acid sequence of a proteolytic fragment of the parent protein in which the novel acetylation site occurs.

An AQUA peptide is preferably at least about 6 amino acids long. The preferred ranged is about 7 to 15 amino acids.

The AQUA method detects and quantifies a target protein in a sample by introducing a known quantity of at least one heavy-isotope labeled peptide standard (which has a unique signature detectable by LC-SRM chromatography) into a digested biological sample. By comparing to the peptide standard, one may readily determines the quantity of a peptide having the same sequence and protein modification(s) in the biological sample. Briefly, the AQUA methodolog_{y} has two stages:(1) peptide internal standard selection and validation; method development; and (2) implementation using validated peptide internal standards to detect and quantify a target protein in a sample. The method is a powerful technique for detecting and quantifying a given peptide/protein within a complex biological mixture, such as a cell lysate, and may be used, e.g., to quantify change in protein acetylation as a result of drug treatment, or to quantify a protein in different biological states.

Generally, to develop a suitable internal standard, a particular peptide (or modified peptide) within a target protein sequence is chosen based on its amino acid sequence and a particular protease for digestion. The peptide is then generated by solid-phase peptide synthesis such that one residue is replaced with that same residue containing stable isotopes (¹³C, ¹⁵N). The result is a peptide that is chemically identical to its native counterpart formed by proteolysis, but is easily distinguishable by MS via a mass shift. A newly synthesized AQUA internal standard peptide is then evaluated by LC-MS/MS. This process provides qualitative information about peptide retention by reverse-phase chromatography, ionization efficiency, and fragmentation via collision-induced dissociation. Informative and abundant fragment ions for sets of native and internal standard peptides are chosen and then specifically monitored in rapid succession as a function of chromatographic retention to form a selected reaction monitoring (LC-SRM) method based on the unique profile of the peptide standard.

The second stage of the AQUA strategy is its implementation to measure the amount of a protein or the modified form of the protein from complex mixtures. Whole cell lysates are typically fractionated by SDS-PAGE gel electrophoresis, and regions of the gel consistent with protein migration are excised. This process is followed by in-gel proteolysis in the presence of the AQUA peptides and LC-SRM analysis. (See Gerber et al. supra.) AQUA peptides are spiked in to the complex peptide mixture obtained by digestion of the whole cell lysate with a proteolytic enzyme and subjected to immunoaffinity purification as described above. The retention time and fragmentation pattern of the native peptide formed by digestion (e.g., trypsinization) is identical to that of the AQUA internal standard peptide determined previously; thus, LC-MS/MS analysis using an SRM experiment results in the highly specific and sensitive measurement of both internal standard and analyte directly from extremely complex peptide mixtures. Because an absolute amount of the AQUA peptide is added (e.g. 250 fmol), the ratio of the areas under the curve can be used to determine the precise expression levels of a protein or acetylated form of a protein in the original cell lysate. In addition, the internal standard is present during in-gel digestion as native peptides are formed, such that peptide extraction efficiency from gel pieces, absolute losses during sample handling (including vacuum centrifugation), and variability during introduction into the LC-MS system do not affect the determined ratio of native and AQUA peptide abundances.

An AQUA peptide standard may be developed for a known acetylation site previously identified by the IAP-LC-MS/MS method within a target protein. One AQUA peptide incorporating the acetylated form of the site, and a second AQUA peptide incorporating the unacetylated form of site may be developed. In this way, the two standards may be used to detect and quantify both the acetylated and unacetylated forms of the site in a biological sample.

Peptide internal standards may also be generated by examining the primary amino acid sequence of a protein and determining the boundaries of peptides produced by protease cleavage. Alternatively, a protein may actually be digested with a protease and a particular peptide fragment produced can then sequenced. Suitable proteases include, but are not limited to, serine proteases (e.g. trypsin, hepsin), metallo proteases (e.g. PUMP1), chymotrypsin, cathepsin, pepsin, thermolysin, carboxypeptidases, etc.

A peptide sequence within a target protein is selected according to one or more criteria to optimize the use of the peptide as an internal standard. Preferably, the size of the peptide is selected to minimize the chances that the peptide sequence will be repeated elsewhere in other non-target proteins. Thus, a peptide is preferably at least about 6 amino acids. The size of the peptide is also optimized to maximize ionization frequency. Thus, peptides longer than about 20 amino acids are not preferred. The preferred ranged is about 7 to 15 amino acids. A peptide sequence is also selected that is not likely to be chemically reactive during mass spectrometry, thus sequences comprising cysteine, tryptophan, or methionine are avoided.

A peptide sequence that is outside a acetylation site may be selected as internal standard to determine the quantity of all forms of the target protein. Alternatively, a peptide encompassing an acetylated site may be selected as internal standard to detect and quantify only the acetylated form of the target protein. Peptide standards for both acetylated form and unacetylated form can be used together, to determine the extent of acetylation in a particular sample.

The peptide is labeled using one or more labeled amino acids (i.e. the label is an actual part of the peptide) or less preferably, labels may be attached after synthesis according to standard methods. Preferably, the label is a mass-altering label selected based on the following considerations: The mass should be unique to shift fragment masses produced by MS analysis to regions of the spectrum with low background; the ion mass signature component is the portion of the labeling moiety that preferably exhibits a unique ion mass signature in MS analysis; the sum of the masses of the constituent atoms of the label is preferably uniquely different than the fragments of all the possible amino acids. As a result, the labeled amino acids and peptides are readily distinguished from unlabeled ones by the ion/mass pattern in the resulting mass spectrum. Preferably, the ion mass signature component imparts a mass to a protein fragment that does not match the residue mass for any of the 20 natural amino acids.

The label should be robust under the fragmentation conditions of MS and not undergo unfavorable fragmentation. Labeling chemistry should be efficient under a range of conditions, particularly denaturing conditions, and the labeled tag preferably remains soluble in the MS buffer system of choice. The label preferably does not suppress the ionization efficiency of the protein and is not chemically reactive. The label may contain a mixture of two or more isotopically distinct species to generate a unique mass spectrometric pattern at each labeled fragment position. Stable isotopes, such as ¹³C, ¹⁵N, ¹⁷O, ¹⁸O, or ³⁴S, are among preferred labels. Pairs of peptide internal standards that incorporate a different isotope label may also be prepared. Preferred amino acid residues into which a heavy isotope label may be incorporated include leucine, proline, valine, and phenylalanine.

Peptide internal standards are characterized according to their mass-to-charge (m/z) ratio, and preferably, also according to their retention time on a chromatographic column (e.g. an HPLC column). Internal standards that co-elute with unlabeled peptides of identical sequence are selected as optimal internal standards. The internal standard is then analyzed by fragmenting the peptide by any suitable means, for example by collision-induced dissociation (CID) using, e.g., argon or helium as a collision gas. The fragments are then analyzed, for example by multi-stage mass spectrometry (MSⁿ) to obtain a fragment ion spectrum, to obtain a peptide fragmentation signature. Preferably, peptide fragments have significant differences in m/z ratios to enable peaks corresponding to each fragment to be well separated, and a signature that is unique for the target peptide is obtained. If a suitable fragment signature is not obtained at the first stage, additional stages of MS are performed until a unique signature is obtained.

Fragment ions in the MS/MS and MS³ spectra are typically highly specific for the peptide of interest, and, in conjunction with LC methods, allow a highly selective means of detecting and quantifying a target peptide/protein in a complex protein mixture, such as a cell lysate, containing many thousands or tens of thousands of proteins. Any biological sample potentially containing a target protein/peptide of interest may be assayed. Crude or partially purified cell extracts are preferably used. Generally, the sample has at least 0.01 mg of protein, typically a concentration of 0.1-10 mg/mL, and may be adjusted to a desired buffer concentration and pH.

A known amount of a labeled peptide internal standard, preferably about 10 femtomoles, corresponding to a target protein to be detected/quantified is then added to a biological sample, such as a cell lysate. The spiked sample is then digested with one or more protease(s) for a suitable time period to allow digestion. A separation is then performed (e.g., by HPLC, reverse-phase HPLC, capillary electrophoresis, ion exchange chromatography, etc.) to isolate the labeled internal standard and its corresponding target peptide from other peptides in the sample. Microcapillary LC is a preferred method.

Each isolated peptide is then examined by monitoring of a selected reaction in the MS. This involves using the prior knowledge gained by the characterization of the peptide internal standard and then requiring the MS to continuously monitor a specific ion in the MS/MS or MSⁿ spectrum for both the peptide of interest and the internal standard. After elution, the area under the curve (AUC) for both peptide standard and target peptide peaks are calculated. The ratio of the two areas provides the absolute quantification that can be normalized for the number of cells used in the analysis and the protein's molecular weight, to provide the precise number of copies of the protein per cell. Further details of the AQUA methodology are described in Gygi et al., and Gerber et al. supra.

Accordingly, AQUA internal peptide standards (heavy-isotope labeled peptides) may be produced, as described above, for any of the 322 novel acetylation sites of the invention (see Table 1/FIG. 2). For example, peptide standards for a given acetylation site (e.g., an AQUA peptide having the sequence ZNF403 (SEQ ID NO: 379), wherein “k” corresponds to phosphorylatable lysine 537 of ZNF403) may be produced for both the acetylated and unacetylated forms of the sequence. Such standards may be used to detect and quantify both acetylated form and unacetylated form of the parent signaling protein (e.g., ZNF403) in a biological sample.

Heavy-isotope labeled equivalents of a acetylation site of the invention, both in acetylated and unacetylated form, can be readily synthesized and their unique MS and LC-SRM signature determined, so that the peptides are validated as AQUA peptides and ready for use in quantification.

The novel acetylation sites of the invention are particularly well suited for development of corresponding AQUA peptides, since the IAP method by which they were identified (see Part A above and Example 1) inherently confirmed that such peptides are in fact produced by enzymatic digestion (e.g., trypsinization) and are in fact suitably fractionated/ionized in MS/MS. Thus, heavy-isotope labeled equivalents of these peptides (both in acetylated and unacetylated form) can be readily synthesized and their unique MS and LC-SRM signature determined, so that the peptides are validated as AQUA peptides and ready for use in quantification experiments.

Accordingly, the invention provides heavy-isotope labeled peptides (AQUA peptides) that may be used for detecting, quantitating, or modulating any of the acetylation sites of the invention (Table 1). For example, an AQUA peptide having the sequence QNTVSkGPFSK (SEQ ID NO: 28), wherein y (Lys 350) may be either acetyl-lysine or lysine, and wherein V=labeled valine (e.g., ¹⁴C)) is provided for the quantification of acetylated (or unacetylated) form of MYST3 (a chromatin or DNA binding/repair/replication protein) in a biological sample.

Example 4 is provided to further illustrate the construction and use, by standard methods described above, of exemplary AQUA peptides provided by the invention. For example, AQUA peptides corresponding to both the acetylated and unacetylated forms of SEQ ID NO: 28 (a trypsin-digested fragment of MYST3, with a lysine 350 acetylation site) may be used to quantify the amount of acetylated MYST3 in a biological sample, e.g., a tumor cell sample or a sample before or after treatment with a therapeutic agent.

Peptides and AQUA peptides provided by the invention will be highly useful in the further study of signal transduction anomalies underlying cancer, including carcinomas. Peptides and AQUA peptides of the invention may also be used for identifying diagnostic/bio-markers of carcinomas, identifying new potential drug targets, and/or monitoring the effects of test therapeutic agents on signaling proteins and pathways.

4. Acetylation Site-Specific Antibodies

In another aspect, the invention discloses acetylation site-specific binding molecules that specifically bind at a novel lysine acetylation site of the invention, and that distinguish between the acetylated and unacetylated forms. In one embodiment, the binding molecule is an antibody or an antigen-binding fragment thereof. The antibody may specifically bind to an amino acid sequence comprising a acetylation site identified in Table 1.

In some embodiments, the antibody or antigen-binding fragment thereof specifically binds the acetylated site. In other embodiments, the antibody or antigen-binding fragment thereof specially binds the unacetylated site. An antibody or antigen-binding fragment thereof specially binds an amino acid sequence comprising a novel lysine acetylation site in Table 1 when it does not significantly bind any other site in the parent protein and does not significantly bind a protein other than the parent protein. An antibody of the invention is sometimes referred to herein as an “acetyl-lysine specific” antibody.

An antibody or antigen-binding fragment thereof specially binds an antigen when the dissociation constant is ≦1 mM, preferably ≦100 nM, and more preferably ≦10 nM.

In some embodiments, the antibody or antigen-binding fragment of the invention binds an amino acid sequence that comprises a novel acetylation site of a protein in Table 1 that is a chormatin or DNA binding/repair/replication protein, enzyme protein, RNA binding protein, transcriptional regulator, translational regulator, ubiquitan conjugating system protein, cytoskeletal protein, adaptor/scaffold protein or receptor/channel/transporter/cell surface protein.

In particularly preferred embodiments, an antibody or antigen-binding fragment thereof of the invention specially binds an amino acid sequence comprising a novel lysine acetylation site shown as a lower case “k” in a sequence listed in Table 1 selected from the group consisting of SEQ ID NOS: 28 (MYST3); 51 (HEMH); 55 (PDA6); 56 (PDA6); 57 (PPIL2); 58 (RSAFD1); 96 (KHSRP); 104 (NSAP1); 143 (eEF1A-1); 145 (EEF1G); 147 (eEF-2); 149 (eIF3-eta); 152 (eIFSA); 155 (RPL10A); 157 (RPL11); 159 (RPL13A); 161 (RPL18a); 162 (RPL24); 164 (RPL5); 166 (RPLP2); 169(RPS5); 172(TUFM); 176 (SAE2); 177 (SKP1A); 178 (UBE2N); 179 (UBE2S); 180 (UCHL5); 41 (gelsolin); 7(NMD3); 80 (SLC25A5); 67 (OTUB1); 225 (DOCK7); 286 (LEREPO4); 316 (NSUN2); and 388 (NSFL1C).

In some embodiments, an antibody or antigen-binding fragment thereof of the invention specifically binds an amino acid sequence comprising any one of the above listed SEQ ID NOs. In some embodiments, an antibody or antigen-binding fragment thereof of the invention especially binds an amino acid sequence comprises a fragment of one of said SEQ ID NOs., wherein the fragment is four to twenty amino acid long and includes the acetylatable lysine.

In certain embodiments, an antibody or antigen-binding fragment thereof of the invention specially binds an amino acid sequence that comprises a peptide produced by proteolysis of the parent protein with a protease wherein said peptide comprises a novel lysine acetylation site of the invention. In some embodiments, the peptides are produced from trypsin digestion of the parent protein. The parent protein comprising the novel lysine acetylation site can be from any species, preferably from a mammal including but not limited to non-human primates, rabbits, mice, rats, goats, cows, sheep, and guinea pigs. In some embodiments, the parent protein is a human protein and the antibody binds an epitope comprising the novel lysine acetylation site shown by a lower case “k” in Column E of Table 1. Such peptides include any one of the SEQ ID NOs.

An antibody of the invention can be an intact, four immunoglobulin chain antibody comprising two heavy chains and two light chains. The heavy chain of the antibody can be of any isotype including IgM, IgG, IgE, IgG, IgA or IgD or sub-isotype including IgG1, IgG2, IgG3, IgG4, IgE1, IgE2, etc. The light chain can be a kappa light chain or a lambda light chain.

Also within the invention are antibody molecules with fewer than 4 chains, including single chain antibodies, Camelid antibodies and the like and components of the antibody, including a heavy chain or a light chain. The term “antibody” (or “antibodies”) refers to all types of immunoglobulins. The term “an antigen-binding fragment of an antibody” refers to any portion of an antibody that retains specific binding of the intact antibody. An exemplary antigen-binding fragment of an antibody is the heavy chain and/or light chain CDR, or the heavy and/or light chain variable region. The term “does not bind,” when appeared in context of an antibody's binding to one acetyl-form (e.g., acetylated form) of a sequence, means that the antibody does not substantially react with the other acetyl-form (e.g., non-acetylated form) of the same sequence. One of skill in the art will appreciate that the expression may be applicable in those instances when (1) an acetyl-specific antibody either does not apparently bind to the non-phospho form of the antigen as ascertained in commonly used experimental detection systems (Western blotting, IHC, Immunofluorescence, etc.); (2) where there is some reactivity with the surrounding amino acid sequence, but that the acetylated residue is an immunodominant feature of the reaction. In cases such as these, there is an apparent difference in affinities for the two sequences. Dilutional analyses of such antibodies indicates that the antibodies apparent affinity for the acetylated form is at least 10-100 fold higher than for the non-acetylated form; or where (3) the acetyl-specific antibody reacts no more than an appropriate control antibody would react under identical experimental conditions. A control antibody preparation might be, for instance, purified immunoglobulin from a pre-immune animal of the same species, an isotype- and species-matched monoclonal antibody. Tests using control antibodies to demonstrate specificity are recognized by one of skill in the art as appropriate and definitive.

In some embodiments an immunoglobulin chain may comprise in order from 5′ to 3′, a variable region and a constant region. The variable region may comprise three complementarity determining regions (CDRs), with interspersed framework (FR) regions for a structure FR1, CDR1, FR2, CDR2, FR3, CDR3 and FR4. Also within the invention are heavy or light chain variable regions, framework regions and CDRs. An antibody of the invention may comprise a heavy chain constant region that comprises some or all of a CH 1 region, hinge, CH2 and CH3 region.

An antibody of the invention may have an binding affinity (K_{D}) of 1×10⁻⁷M or less. In other embodiments, the antibody binds with a K_{D} of 1×10⁻⁸ M, 1×10⁻⁹ M, 1×10⁻¹⁰M, 1×10⁻¹¹ M, 1×10⁻¹²M or less. In certain embodiments, the K_{D} is 1 pM to 500 pM, between 500 pM to 1 μM, between 1 μM to 100 nM, or between 100 mM to 10 nM.

Antibodies of the invention can be derived from any species of animal, preferably a mammal. Non-limiting exemplary natural antibodies include antibodies derived from human, chicken, goats, and rodents (e.g., rats, mice, hamsters and rabbits), including transgenic rodents genetically engineered to produce human antibodies (see, e.g., Lonberg et al., WO93/12227; U.S. Pat. No. 5,545,806; and Kucherlapati, et al., WO91/10741; U.S. Pat. No. 6,150,584, which are herein incorporated by reference in their entirety). Natural antibodies are the antibodies produced by a host animal. “Genetically altered antibodies” refer to antibodies wherein the amino acid sequence has been varied from that of a native antibody. Because of the relevance of recombinant DNA techniques to this application, one need not be confined to the sequences of amino acids found in natural antibodies; antibodies can be redesigned to obtain desired characteristics. The possible variations are many and range from the changing of just one or a few amino acids to the complete redesign of, for example, the variable or constant region. Changes in the constant region will, in general, be made in order to improve or alter characteristics, such as complement fixation, interaction with membranes and other effector functions. Changes in the variable region will be made in order to improve the antigen binding characteristics.

The antibodies of the invention include antibodies of any isotype including IgM, IgG, IgD, IgA and IgE, and any sub-isotype, including IgG1, IgG2a, IgG2b, IgG3 and IgG4, IgE1, IgE2 etc. The light chains of the antibodies can either be kappa light chains or lambda light chains.

Antibodies disclosed in the invention may be polyclonal or monoclonal. As used herein, the term “epitope” refers to the smallest portion of a protein capable of selectively binding to the antigen binding site of an antibody. It is well accepted by those skilled in the art that the minimal size of a protein epitope capable of selectively binding to the antigen binding site of an antibody is about five or six to seven amino acids.

Other antibodies specifically contemplated are oligoclonal antibodies. As used herein, the phrase “oligoclonal antibodies” refers to a predetermined mixture of distinct monoclonal antibodies. See, e.g., PCT publication WO 95/20401; U.S. Pat. Nos. 5,789,208 and 6,335,163. In one embodiment, oligoclonal antibodies consisting of a predetermined mixture of antibodies against one or more epitopes are generated in a single cell. In other embodiments, oligoclonal antibodies comprise a plurality of heavy chains capable of pairing with a common light chain to generate antibodies with multiple specificities (e.g., PCT publication WO 04/009618). Oligoclonal antibodies are particularly useful when it is desired to target multiple epitopes on a single target molecule. In view of the assays and epitopes disclosed herein, those skilled in the art can generate or select antibodies or mixtures of antibodies that are applicable for an intended purpose and desired need.

Recombinant antibodies against the acetylation sites identified in the invention are also included in the present application. These recombinant antibodies have the same amino acid sequence as the natural antibodies or have altered amino acid sequences of the natural antibodies in the present application. They can be made in any expression systems including both prokaryotic and eukaryotic expression systems or using phage display methods (see, e.g., Dower et al., WO91/17271 and McCafferty et al., WO92/01047; U.S. Pat. No. 5,969,108, which are herein incorporated by reference in their entirety).

Antibodies can be engineered in numerous ways. They can be made as single-chain antibodies (including small modular immunopharmaceuticals or SMIPs™), Fab and F(ab′)₂ fragments, etc. Antibodies can be humanized, chimerized, deimmunized, or fully human. Numerous publications set forth the many types of antibodies and the methods of engineering such antibodies. For example, see U.S. Pat. Nos. 6,355,245; 6,180,370; 5,693,762; 6,407,213; 6,548,640; 5,565,332; 5,225,539; 6,103,889; and 5,260,203.

The genetically altered antibodies should be functionally equivalent to the above-mentioned natural antibodies. In certain embodiments, modified antibodies provide improved stability or/and therapeutic efficacy. Examples of modified antibodies include those with conservative substitutions of amino acid residues, and one or more deletions or additions of amino acids that do not significantly deleteriously alter the antigen binding utility. Substitutions can range from changing or modifying one or more amino acid residues to complete redesign of a region as long as the therapeutic utility is maintained. Antibodies of this application can be modified post-translationally (e.g., phosphorylation, and/or acetylation) or can be modified synthetically (e.g., the attachment of a labeling group).

Antibodies with engineered or variant constant or Fc regions can be useful in modulating effector functions, such as, for example, antigen-dependent cytotoxicity (ADCC) and complement-dependent cytotoxicity (CDC). Such antibodies with engineered or variant constant or Fc regions may be useful in instances where a parent singling protein (Table 1) is expressed in normal tissue; variant antibodies without effector function in these instances may elicit the desired therapeutic response while not damaging normal tissue. Accordingly, certain aspects and methods of the present disclosure relate to antibodies with altered effector functions that comprise one or more amino acid substitutions, insertions, and/or deletions.

In certain embodiments, genetically altered antibodies are chimeric antibodies and humanized antibodies.

The chimeric antibody is an antibody having portions derived from different antibodies. For example, a chimeric antibody may have a variable region and a constant region derived from two different antibodies. The donor antibodies may be from different species. In certain embodiments, the variable region of a chimeric antibody is non-human, e.g., murine, and the constant region is human.

The genetically altered antibodies used in the invention include CDR grafted humanized antibodies. In one embodiment, the humanized antibody comprises heavy and/or light chain CDRs of a non-human donor immunoglobulin and heavy chain and light chain frameworks and constant regions of a human acceptor immunoglobulin. The method of making humanized antibody is disclosed in U.S. Pat. Nos: 5,530,101; 5,585,089; 5,693,761; 5,693,762; and 6,180,370 each of which is incorporated herein by reference in its entirety.

Antigen-binding fragments of the antibodies of the invention, which retain the binding specificity of the intact antibody, are also included in the invention. Examples of these antigen-binding fragments include, but are not limited to, partial or full heavy chains or light chains, variable regions, or CDR regions of any acetylation site-specific antibodies described herein.

In one embodiment of the application, the antibody fragments are truncated chains (truncated at the carboxyl end). In certain embodiments, these truncated chains possess one or more immunoglobulin activities (e.g., complement fixation activity). Examples of truncated chains include, but are not limited to, Fab fragments (consisting of the VL, VH, CL and CHI domains); Fd fragments (consisting of the VH and CH1 domains); Fv fragments (consisting of VL and VH domains of a single chain of an antibody); dAb fragments (consisting of a VH domain); isolated CDR regions; (Fab′)₂ fragments, bivalent fragments (comprising two Fab fragments linked by a disulphide bridge at the hinge region). The truncated chains can be produced by conventional biochemical techniques, such as enzyme cleavage, or recombinant DNA techniques, each of which is known in the art. These polypeptide fragments may be produced by proteolytic cleavage of intact antibodies by methods well known in the art, or by inserting stop codons at the desired locations in the vectors using site-directed mutagenesis, such as after CH1 to produce Fab fragments or after the hinge region to produce (Fab′)₂ fragments. Single chain antibodies may be produced by joining VL- and VH-coding regions with a DNA that encodes a peptide linker connecting the VL and VH protein fragments

Papain digestion of antibodies produces two identical antigen-binding fragments, called “Fab” fragments, each with a single antigen-binding site, and a residual “Fc” fragment, whose name reflects its ability to crystallize readily. Pepsin treatment of an antibody yields an F(ab′)₂ fragment that has two antigen-combining sites and is still capable of cross-linking antigen.

“Fv” usually refers to the minimum antibody fragment that contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the CDRs confer antigen-binding specificity to the antibody. However, even a single variable domain (or half of an Fv comprising three CDRs specific for an antigen) has the ability to recognize and bind antigen, although likely at a lower affinity than the entire binding site.

Thus, in certain embodiments, the antibodies of the application may comprise 1, 2, 3, 4, 5, 6, or more CDRs that recognize the acetylation sites identified in Column E of Table 1.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab' fragments that have hinge cysteines between them. Other chemical couplings of antibody fragments are also known.

“Single-chain Fv” or “scFv” antibody fragments comprise the V_{H} and V_{L} domains of an antibody, wherein these domains are present in a single polypeptide chain. In certain embodiments, the Fv polypeptide further comprises a polypeptide linker between the V_{H} and V_{L} domains that enables the scFv to form the desired structure for antigen binding. For a review of scFv see Pluckthun in The Pharmacology of Monoclonal Antibodies, vol. 113, Rosenburg and Moore, eds. (Springer-Verlag: New York, 1994), pp. 269-315.

SMIPs are a class of single-chain peptides engineered to include a target binding region and effector domain (CH2 and CH3 domains). See, e.g., U.S. Patent Application Publication No. 20050238646. The target binding region may be derived from the variable region or CDRs of an antibody, e.g., a acetylation site-specific antibody of the application. Alternatively, the target binding region is derived from a protein that binds a acetylation site.

Bispecific antibodies may be monoclonal, human or humanized antibodies that have binding specificities for at least two different antigens. In the present case, one of the binding specificities is for the acetylation site, the other one is for any other antigen, such as for example, a cell-surface protein or receptor or receptor subunit. Alternatively, a therapeutic agent may be placed on one arm. The therapeutic agent can be a drug, toxin, enzyme, DNA, radionuclide, etc.

In some embodiments, the antigen-binding fragment can be a diabody. The term “diabody” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90: 6444-6448 (1993).

Camelid antibodies refer to a unique type of antibodies that are devoid of light chain, initially discovered from animals of the camelid family. The heavy chains of these so-called heavy-chain antibodies bind their antigen by one single domain, the variable domain of the heavy immunoglobulin chain, referred to as VHH. VHHs show homology with the variable domain of heavy chains of the human VHIII family. The VHHs obtained from an immunized camel, dromedary, or llama have a number of advantages, such as effective production in microorganisms such as Saccharomyces cerevisiae.

In certain embodiments, single chain antibodies, and chimeric, humanized or primatized (CDR-grafted) antibodies, as well as chimeric or CDR-grafted single chain antibodies, comprising portions derived from different species, are also encompassed by the present disclosure as antigen-binding fragments of an antibody. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., U.S. Pat. Nos. 4,816,567 and 6,331,415; U.S. Pat. No. 4,816,397; European Patent No. 0,120,694; WO 86/01533; European Patent No. 0,194,276 B1; U.S. Pat. No. 5,225,539; and European Patent No. 0,239,400 B1. See also, Newman et al., BioTechnology, 10: 1455-1460 (1992), regarding primatized antibody. See, e.g., Ladner et al., U.S. Pat. No. 4,946,778; and Bird et al., Science, 242: 423-426 (1988)), regarding single chain antibodies.

In addition, functional fragments of antibodies, including fragments of chimeric, humanized, primatized or single chain antibodies, can also be produced. Functional fragments of the subject antibodies retain at least one binding function and/or modulation function of the full-length antibody from which they are derived.

Since the immunoglobulin-related genes contain separate functional regions, each having one or more distinct biological activities, the genes of the antibody fragments may be fused to functional regions from other genes (e.g., enzymes, U.S. Pat. No. 5,004,692, which is incorporated by reference in its entirety) to produce fusion proteins or conjugates having novel properties.

Non-immunoglobulin binding polypeptides are also contemplated. For example, CDRs from an antibody disclosed herein may be inserted into a suitable non-immunoglobulin scaffold to create a non-immunoglobulin binding polypeptide. Suitable candidate scaffold structures may be derived from, for example, members of fibronectin type III and cadherin superfamilies.

Also contemplated are other equivalent non-antibody molecules, such as protein binding domains or aptamers, which bind, in a phospho-specific manner, to an amino acid sequence comprising a novel acetylation site of the invention. See, e.g., Neuberger et al., Nature 312: 604 (1984). Aptamers are oligonucleic acid or peptide molecules that bind a specific target molecule. DNA or RNA aptamers are typically short oligonucleotides, engineered through repeated rounds of selection to bind to a molecular target. Peptide aptamers typically consist of a variable peptide loop attached at both ends to a protein scaffold. This double structural constraint generally increases the binding affinity of the peptide aptamer to levels comparable to an antibody (nanomolar range).

The invention also discloses the use of the acetylation site-specific antibodies with immunotoxins. Conjugates that are immunotoxins including antibodies have been widely described in the art. The toxins may be coupled to the antibodies by conventional coupling techniques or immunotoxins containing protein toxin portions can be produced as fusion proteins. In certain embodiments, antibody conjugates may comprise stable linkers and may release cytotoxic agents inside cells (see U.S. Pat. Nos. 6,867,007 and 6,884,869). The conjugates of the present application can be used in a corresponding way to obtain such immunotoxins. Illustrative of such immunotoxins are those described by Byers et al., Seminars Cell Biol 2:59-70 (1991) and by Fanger et al., Immunol Today 12:51-54 (1991). Exemplary immunotoxins include radiotherapeutic agents, ribosome-inactivating proteins (RIPs), chemotherapeutic agents, toxic peptides, or toxic proteins.

The acetylation site-specific antibodies disclosed in the invention may be used singly or in combination. The antibodies may also be used in an array format for high throughput uses. An antibody microarray is a collection of immobolized antibodies, typically spotted and fixed on a solid surface (such as glass, plastic and silicon chip).

In another aspect, the antibodies of the invention modulate at least one, or all, biological activities of a parent protein identified in Column A of Table 1. The biological activities of a parent protein identified in Column A of Table 1 include: 1) ligand binding activities (for instance, these neutralizing antibodies may be capable of competing with or completely blocking the binding of a parent signaling protein to at least one, or all, of its ligands; 2) signaling transduction activities, such as receptor dimerization, or lysine acetylation; and 3) cellular responses induced by a parent signaling protein, such as oncogenic activities (e.g., cancer cell proliferation mediated by a parent signaling protein), and/or angiogenic activities.

In certain embodiments, the antibodies of the invention may have at least one activity selected from the group consisting of: 1) inhibiting cancer cell growth or proliferation; 2) inhibiting cancer cell survival; 3) inhibiting angiogenesis; 4) inhibiting cancer cell metastasis, adhesion, migration or invasion; 5) inducing apoptosis of cancer cells; 6) incorporating a toxic conjugate; and 7) acting as a diagnostic marker.

In certain embodiments, the acetylation site specific antibodies disclosed in the invention are especially indicated for diagnostic and therapeutic applications as described herein. Accordingly, the antibodies may be used in therapies, including combination therapies, in the diagnosis and prognosis of disease, as well as in the monitoring of disease progression. The invention, thus, further includes compositions comprising one or more embodiments of an antibody or an antigen binding portion of the invention as described herein. The composition may further comprise a pharmaceutically acceptable carrier. The composition may comprise two or more antibodies or antigen-binding portions, each with specificity for a different novel lysine acetylation site of the invention or two or more different antibodies or antigen-binding portions all of which are specific for the same novel lysine acetylation site of the invention. A composition of the invention may comprise one or more antibodies or antigen-binding portions of the invention and one or more additional reagents, diagnostic agents or therapeutic agents.

The present application provides for the polynucleotide molecules encoding the antibodies and antibody fragments and their analogs described herein. Because of the degeneracy of the genetic code, a variety of nucleic acid sequences encode each antibody amino acid sequence. The desired nucleic acid sequences can be produced by de novo solid-phase DNA synthesis or by PCR mutagenesis of an earlier prepared variant of the desired polynucleotide. In one embodiment, the codons that are used comprise those that are typical for human or mouse (see, e.g., Nakamura, Y., Nucleic Acids Res. 28: 292 (2000)).

The invention also provides immortalized cell lines that produce an antibody of the invention. For example, hybridoma clones, constructed as described above, that produce monoclonal antibodies to the targeted signaling protein acetylation sties disclosed herein are also provided. Similarly, the invention includes recombinant cells producing an antibody of the invention, which cells may be constructed by well known techniques; for example the antigen combining site of the monoclonal antibody can be cloned by PCR and single-chain antibodies produced as phage-displayed recombinant antibodies or soluble antibodies in E. coli (see, e.g., ANTIBODY ENGINEERING PROTOCOLS, 1995, Humana Press, Sudhir Paul editor.)

### 5. Methods of Making Acetylation Site-Specific Antibodies

In another aspect, the invention provides a method for making acetylation site-specific antibodies.

Polyclonal antibodies of the invention may be produced according to standard techniques by immunizing a suitable animal (e.g., rabbit, goat, etc.) with an antigen comprising a novel lysine acetylation site of the invention. (i.e. a acetylation site shown in Table 1) in either the acetylated or unacetylated state, depending upon the desired specificity of the antibody, collecting immune serum from the animal, and separating the polyclonal antibodies from the immune serum, in accordance with known procedures and screening and isolating a polyclonal antibody specific for the novel lysine acetylation site of interest as further described below. Methods for immunizing non-human animals such as mice, rats, sheep, goats, pigs, cattle and horses are well known in the art. See, e.g., Harlow and Lane, Antibodies: A Laboratory Manual, New York: Cold Spring Harbor Press, 1990.

The immunogen may be the full length protein or a peptide comprising the novel lysine acetylation site of interest. In some embodiments the immunogen is a peptide of from 7 to 20 amino acids in length, preferably about 8 to 17 amino acids in length. In some embodiments, the peptide antigen desirably will comprise about 3 to 8 amino acids on each side of the phosphorylatable lysine. In yet other embodiments, the peptide antigen desirably will comprise four or more amino acids flanking each side of the phosphorylatable amino acid and encompassing it. Peptide antigens suitable for producing antibodies of the invention may be designed, constructed and employed in accordance with well-known techniques. See, e.g., Antibodies: A Laboratory Manual, Chapter 5, p. 75-76, Harlow & Lane Eds., Cold Spring Harbor Laboratory (1988); Czemik, Methods In Enzymology, 201: 264-283 (1991); Merrifield, J. Am. Chem. Soc. 85: 21-49 (1962)).

Suitable peptide antigens may comprise all or partial sequence of a trypsin-digested fragment as set forth in Column E of Table 1/FIG. 2. Suitable peptide antigens may also comprise all or partial sequence of a peptide fragment produced by another protease digestion.

Preferred immunogens are those that comprise a novel acetylation site of a protein in Table 1 that is a chormatin or DNA binding/repair/replication protein, enzyme protein, RNA binding protein, transcriptional regulator, translational regulator, ubiquitan conjugating system protein, cytoskeletal protein, adaptor/scaffold protein or receptor/channeUtransporter/cell surface protein. In some embodiments, the peptide immunogen is an AQUA peptide, for example, any one of the sequences listed in column E of Table one and FIG. 2.

Particularly preferred immunogens are peptides comprising any one of the novel lysine acetylation site shown as a lower case “k” in a sequence listed in Table I selected from the group consisting of SEQ ID NOS: 28 (MYST3); 51 (HEMH); 55 (PDA6); 56 (PDA6); 57 (PPIL2); 58 (RSAFD1); 96 (KHSRP); 104 (NSAP1); 143 (eEF1A-1); 145 (EEFIG); 147 (eEF-2); 149 (eIF3-eta); 152 (eIF5A); 155 (RPL10A); 157 (RPL 11); 159 (RPL13A); 161 (RPL18a); 162 (RPL24); 164 (RPL5); 166 (RPLP2); 169(RPS5); 172(TUFM); 176 (SAE2); 177 (SKP1A); 178 (UBE2N); 179 (UBE2S); 180 (UCHL5); 41 (gelsolin); 7(NMD3); 80 (SLC25A5); 67 (OTUB1); 225 (DOCK7); 286 (LEREPO4); 316 (NSUN2); and 388 (NSFL1C).

In some embodiments the immunogen is administered with an adjuvant. Suitable adjuvants will be well known to those of skill in the art. Exemplary adjuvants include complete or incomplete Freund's adjuvant, RIBI (muramyl dipeptides) or ISCOM (immunostimulating complexes).

For example, a peptide antigen comprising the novel receptor lysine kinase acetylation site in SEQ ID NO: 4 shown by the lower case “k” in Table 1 may be used to produce antibodies that specifically bind the novel lysine acetylation site.

When the above-described methods are used for producing polyclonal antibodies, following immunization, the polyclonal antibodies which secreted into the bloodstream can be recovered using known techniques. Purified forms of these antibodies can, of course, be readily prepared by standard purification techniques, such as for example, affinity chromatography with Protein A, anti-immunoglobulin, or the antigen itself. In any case, in order to monitor the success of immunization, the antibody levels with respect to the antigen in serum will be monitored using standard techniques such as ELISA, RIA and the like.

Monoclonal antibodies of the invention may be produced by any of a number of means that are well-known in the art. In some embodiments, antibody-producing B cells are isolated from an animal immunized with a peptide antigen as described above. The B cells may be from the spleen, lymph nodes or peripheral blood. Individual B cells are isolated and screened as described below to identify cells producing an antibody specific for the novel lysine acetylation site of interest. Identified cells are then cultured to produce a monoclonal antibody of the invention.

Alternatively, a monoclonal acetylation site-specific antibody of the invention may be produced using standard hybridoma technology, in a hybridoma cell line according to the well-known technique of Kohler and Milstein. See Nature 265: 495-97 (1975); Kohler and Milstein, Eur. J. Immunol. 6: 511 (1976); see also, Current Protocols in Molecular Biology, Ausubel et al. Eds. (1989). Monoclonal antibodies so produced are highly specific, and improve the selectivity and specificity of diagnostic assay methods provided by the invention. For example, a solution containing the appropriate antigen may be injected into a mouse or other species and, after a sufficient time (in keeping with conventional techniques), the animal is sacrificed and spleen cells obtained. The spleen cells are then immortalized by any of a number of standard means. Methods of immortalizing cells include, but are not limited to, transfecting them with oncogenes, infecting them with an oncogenic virus and cultivating them under conditions that select for immortalized cells, subjecting them to carcinogenic or mutating compounds, fusing them with an immortalized cell, e.g., a myeloma cell, and inactivating a tumor suppressor gene. See, e.g., Harlow and Lane, supra. If fusion with myeloma cells is used, the myeloma cells preferably do not secrete immunoglobulin polypeptides (a non-secretory cell line). Typically the antibody producing cell and the immortalized cell (such as but not limited to myeloma cells) with which it is fused are from the same species. Rabbit fusion hybridomas, for example, may be produced as described in U.S Pat. No. 5,675,063, C. Knight, Issued Oct. 7, 1997. The immortalized antibody producing cells, such as hybridoma cells, are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

The invention also encompasses antibody-producing cells and cell lines, such as hybridomas, as described above.

Polyclonal or monoclonal antibodies may also be obtained through in vitro immunization. For example, phage display techniques can be used to provide libraries containing a repertoire of antibodies with varying affinities for a particular antigen. Techniques for the identification of high affinity human antibodies from such libraries are described by Griffiths et al., (1994) EMBO J., 13:3245-3260; Nissim et al., ibid, pp. 692-698 and by Griffiths et al., ibid, 12:725-734, which.are incorporated by reference.

The antibodies may be produced recombinantly using methods well known in the art for example, according to the methods disclosed in U.S. Pat. No. 4,349,893 (Reading) or U.S. Pat. No. 4,816,567 (Cabilly et al.) The antibodies may also be chemically constructed by specific antibodies made according to the method disclosed in U.S. Pat. No. 4,676,980 (Segel et al.)

Once a desired acetylation site-specific antibody is identified, polynucleotides encoding the antibody, such as heavy, light chains or both (or single chains in the case of a single chain antibody) or portions thereof such as those encoding the variable region, may be cloned and isolated from antibody-producing cells using means that are well known in the art. For example, the antigen combining site of the monoclonal antibody can be cloned by PCR and single-chain antibodies produced as phage-displayed recombinant antibodies or soluble antibodies in E. coli (see, e.g., Antibody Engineering Protocols, 1995, Humana Press, Sudhir Paul editor.)

Accordingly, in a further aspect, the invention provides such nucleic acids encoding the heavy chain, the light chain, a variable region, a framework region or a CDR of an antibody of the invention. In some embodiments, the nucleic acids are operably linked to expression control sequences. The invention, thus, also provides vectors and expression control sequences useful for the recombinant expression of an antibody or antigen-binding portion thereof of the invention. Those of skill in the art will be able to choose vectors and expression systems that are suitable for the host cell in which the antibody or antigen-binding portion is to be expressed.

Monoclonal antibodies of the invention may be produced recombinantly by expressing the encoding nucleic acids in a suitable host cell under suitable conditions. Accordingly, the invention further provides host cells comprising the nucleic acids and vectors described above.

Monoclonal Fab fragments may also be produced in Escherichia coli by recombinant techniques known to those skilled in the art. See, e.g., W. Huse, Science 246: 1275-81 (1989); Mullinax et al., Proc. Nat'l. Acad. Sci. 87: 8095 (1990).

If monoclonal antibodies of a single desired isotype are preferred for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Acad. Sci., 82: 8653 (1985); Spira et al., J. Immunol. Methods, 74: 307 (1984)). Alternatively, the isotype of a monoclonal antibody with desirable propertied can be changed using antibody engineering techniques that are well-known in the art.

Acetylation site-specific antibodies of the invention, whether polyclonal or monoclonal, may be screened for epitope and phospho-specificity according to standard techniques. See, e.g., Czernik et al., Methods in Enzymology, 201: 264-283 (1991). For example, the antibodies may be screened against the acetylated and/or unacetylated peptide library by ELISA to ensure specificity for both the desired antigen (i.e. that epitope including an acetylation site of the invention and for reactivity only with the acetylated (or unacetylated) form of the antigen. Peptide competition assays may be carried out to confirm lack of reactivity with other phospho-epitopes on the parent protein. The antibodies may also be tested by Western blotting against cell preparations containing the parent signaling protein, e.g., cell lines over-expressing the parent protein, to confirm reactivity with the desired acetylated epitope/target.

Specificity against the desired acetylated epitope may also be examined by constructing mutants lacking phosphorylatable residues at positions outside the desired epitope that are known to be acetylated, or by mutating the desired phospho-epitope and confirming lack of reactivity. Acetylation site-specific antibodies of the invention may exhibit some limited cross-reactivity to related epitopes in non-target proteins. This is not unexpected as most antibodies exhibit some degree of cross-reactivity, and anti-peptide antibodies will often cross-react with epitopes having high homology to the immunizing peptide. See, e.g., Czernik, supra. Cross-reactivity with non-target proteins is readily characterized by Western blotting alongside markers of known molecular weight. Amino acid sequences of cross-reacting proteins may be examined to identify acetylation sites with flanking sequences that are highly homologous to that of a acetylation site of the invention.

In certain cases, polyclonal antisera may exhibit some undesirable general cross-reactivity to acetyl-lysine itself, which may be removed by further purification of antisera, e.g., over an acetyl-lysine column. Antibodies of the invention specifically bind their target protein (i.e. a protein listed in Column A of Table 1) only when acetylated (or only when not acetylated, as the case may be) at the site disclosed in corresponding Columns D/E, and do not (substantially) bind to the other form (as compared to the form for which the antibody is specific).

Antibodies may be further characterized via immunohistochemical (IHC) staining using normal and diseased tissues to examine acetylation and activation state and level of a acetylation site in diseased tissue. IHC may be carried out according to well-known techniques. See, e.g., Antibodies: A Laboratory Manual, Chapter 10, Harlow & Lane Eds., Cold Spring Harbor Laboratory (1988). Briefly, paraffin-embedded tissue’(e.g., tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

Antibodies may be further characterized by flow cytometry carried out according to standard methods. See Chow et al., Cytometry (Communications in Clinical Cytometry) 46: 72-78 (2001). Briefly and by way of example, the following protocol for cytometric analysis may be employed: samples may be centrifuged on Ficoll gradients to remove lysed erythrocytes and cell debris. Adherring cells may be scrapped off plates and washed with PBS. Cells may then be fixed with 2% paraformaldehyde for 10 minutes at 37° C. followed by permeabilization in 90% methanol for 30 minutes on ice. Cells may then be stained with the primary acetylation site-specific antibody of the invention (which detects a parent signaling protein enumerated in Table 1), washed and labeled with a fluorescent-labeled secondary antibody. Additional fluorochrome-conjugated marker antibodies (e.g., CD45, CD34) may also be added at this time to aid in the subsequent identification of specific hematopoietic cell types. The cells would then be analyzed on a flow cytometer (e.g. a Beckman Coulter FC500) according to the specific protocols of the instrument used.

Antibodies of the invention may also be advantageously conjugated to fluorescent dyes (e.g. Alexa488, PE) for use in multi-parametric analyses along with other signal transduction (phospho-CrkL, phospho-Erk ½) and/or cell marker (CD34) antibodies.

Acetylation site-specific antibodies of the invention may specifically bind to a signaling protein or polypeptide listed in Table 1 only when acetylated at the specified lysine residue, but are not limited only to binding to the listed signaling proteins of human species, per se. The invention includes antibodies that also bind conserved and highly homologous or identical acetylation sites in respective signaling proteins from other species (e.g., mouse, rat, monkey, yeast), in addition to binding the acetylation site of the human homologue. The term “homologous” refers to two or more sequences or subsequences that have at least about 85%, at least 90%, at least 95%, or higher nucleotide or amino acid residue identity, when compared and aligned for maximum correspondence, as measured using sequence comparison method (e.g., BLAST) and/or by visual inspection. Highly homologous or identical sites conserved in other species can readily be identified by standard sequence comparisons (such as BLAST).

Methods for making bispecific antibodies are within the purview of those skilled in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Antibody variable domains with the desired binding specificities (antibody-antigen combining sites) can be fused to immunoglobulin constant domain sequences. In certain embodiments, the fusion is with an immunoglobulin heavy-chain constant domain, including at least part of the hinge, CH2, and CH3 regions. DNAs encoding the immunoglobulin heavy-chain fusions and, if desired, the immunoglobulin light chain, are inserted into separate expression vectors, and are co-transfected into a suitable host organism. For further details of illustrative currently known methods for generating bispecific antibodies see, for example, Suresh et al., Methods in Enzymology, 121:210 (1986); WO 96127011; Brennan et al., Science 229:81 (1985); Shalaby et al., J. Exp. Med. 175:217-225 (1992); Kostelny et al., J. Immunol. 148(5):1547-1553 (1992); Hollinger et al., Proc. Natl. Acad. Sci. USA 90:6444-6448 (1993); Gruber et al., J. Immunol. 152:5368 (1994); and Tutt et al., J. Immunol. 147:60 (1991). Bispecific antibodies also include cross-linked or heteroconjugate antibodies. Heteroconjugate antibodies may be made using any convenient cross-linking methods. Suitable cross-linking agents are well known in the art, and are disclosed in U.S. Pat. No. 4,676,980, along with a number of cross-linking techniques.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins may be linked to the Fab′ portions of two different antibodies by gene fusion. The antibody homodimers may be reduced at the hinge region to form monomers and then re-oxidized to form the antibody heterodimers. This method can also be utilized for the production of antibody homodimers. A strategy for making bispecific antibody fragments by the use of single-chain Fv (scFv) dimers has also been reported. See Gruber at al., J. Immunol., 152:5368 (1994). Alternatively, the antibodies can be “linear antibodies” as described in Zapata et al. Protein Eng. 8(10):1057-1062 (1995). Briefly, these antibodies comprise a pair of tandem Fd segments (V_{H}-C_{H}1-V_{H}-C_{H}1) which form a pair of antigen binding regions. Linear antibodies can be bispecific or monospecific. 101831 To produce the chimeric antibodies, the portions derived from two different species (e.g., human constant region and murine variable or binding region) can be joined together chemically by conventional techniques or can be prepared as single contiguous proteins using genetic engineering techniques. The DNA molecules encoding the proteins of both the light chain and heavy chain portions of the chimeric antibody can be expressed as contiguous proteins. The method of making chimeric antibodies is disclosed in U.S. Pat. No. 5,677,427; U.S. Pat. No. 6,120,767; and U.S. Pat. No. 6,329,508, each of which is incorporated by reference in its entirety.

Fully human antibodies may be produced by a variety of techniques. One example is trioma methodology. The basic approach and an exemplary cell fusion partner, SPAZ-4, for use in this approach have been described by Oestberg et al., Hybridoma 2:361-367 (1983); Oestberg, U.S. Pat. No. 4,634,664; and Engleman et al., U.S. Pat. No. 4,634,666 (each of which is incorporated by reference in its entirety).

Human antibodies can also be produced from non-human transgenic animals having transgenes encoding at least a segment of the human immunoglobulin locus. The production and properties of animals having these properties are described in detail by, see, e.g., Lonberg et al., WO93/12227; U.S. Pat. No. 5,545,806; and Kucherlapati, et al., WO91/10741; U.S. Pat. No. 6,150,584, which are herein incorporated by reference in their entirety.

Various recombinant antibody library technologies may also be utilized to produce fully human antibodies. For example, one approach is to screen a DNA library from human B cells according to the general protocol outlined by Huse et al., Science 246:1275-1281 (1989). The protocol described by Huse is rendered more efficient in combination with phage-display technology. See, e.g., Dower et al., WO 91/17271 and McCafferty et al., WO 92/01047; U.S. Pat. No. 5,969,108, (each of which is incorporated by reference in its entirety).

Eukaryotic ribosome can also be used as means to display a library of antibodies and isolate the binding human antibodies by screening against the target antigen, as described in Coia G, et al., J. Immunol. Methods 1: 254 (1-2):191-7 (2001); Hanes J. et al., Nat. Biotechnol. 18(12):1287-92 (2000); Proc. Natl. Acad. Sci. U.S.A. 95(24):14130-5 (1998); Proc. Natl. Acad. Sci. U.S.A. 94(10):4937-42 (1997), each which is incorporated by reference in its entirety.

The yeast system is also suitable for screening mammalian cell-surface or secreted proteins, such as antibodies. Antibody libraries may be displayed on the surface of yeast cells for the purpose of obtaining the human antibodies against a target antigen. This approach is described by Yeung, et al., Biotechnol. Prog. 18(2):212-20 (2002); Boeder, E. T., et al., Nat. Biotechnol. 15(6):553-7 (1997), each of which is herein incorporated by reference in its entirety. Alternatively, human antibody libraries may be expressed intracellularly and screened via the yeast two-hybrid system (WO0200729A2, which is incorporated by reference in its entirety).

Recombinant DNA techniques can be used to produce the recombinant acetylation site-specific antibodies described herein, as well as the chimeric or humanized acetylation site-specific antibodies, or any other genetically-altered antibodies and the fragments or conjugate thereof in any expression systems including both prokaryotic and eukaryotic expression systems, such as bacteria, yeast, insect cells, plant cells, mammalian cells (for example, NS0 cells).

Once produced, the whole antibodies, their dimers, individual light and heavy chains, or other immunoglobulin forms of the present application can be purified according to standard procedures of the art, including ammonium sulfate precipitation, affinity columns, column chromatography, gel electrophoresis and the like (see, generally, Scopes, R., Protein Purification (Springer-Verlag, N.Y., 1982)). Once purified, partially or to homogeneity as desired, the polypeptides may then be used therapeutically (including extracorporeally) or in developing and performing assay procedures, immunofluorescent staining, and the like. (See, generally, Immunological Methods, Vols. I and II (Lefkovits and Pernis, eds., Academic Press, NY, 1979 and 1981).

### 6. Therapeutic Uses

In a further aspect, the invention provides methods and compositions for therapeutic uses of the peptides or proteins comprising a acetylation site of the invention, and acetylation site-specific antibodies of the invention.

In one embodiment, the invention provides for a method of treating or preventing cancer in a subject, wherein the cancer is associated with the acetylation state of a novel acetylation site in Table 1, whether acetylated or deacetylated, comprising: administering to a subject in need thereof a therapeutically effective amount of a peptide comprising a novel acetylation site (Table 1) and/or an antibody or antigen-binding fragment thereof that specifically bind a novel acetylation site of the invention (Table 1). The antibodies maybe full-length antibodies, genetically engineered antibodies, antibody fragments, and antibody conjugates of the invention.

The term “subject” refers to a vertebrate, such as for example, a mammal, or a human. Although present application are primarily concerned with the treatment of human subjects, the disclosed methods may also be used for the treatment of other mammalian subjects such as dogs and cats for veterinary purposes.

In one aspect, the disclosure provides a method of treating cancer in which a peptide or an antibody that reduces at least one biological activity of a targeted signaling protein is administered to a subject. For example, the peptide or the antibody administered may disrupt or modulate the interaction of the target signaling protein with its ligand. Alternatively, the peptide or the antibody may interfere with, thereby reducing, the down-stream signal transduction of the parent signaling protein. An antibody that specifically binds the novel lysine acetylation site only when the lysine is acetylated, and that does not substantially bind to the same sequence when the lysine is not acetylated, thereby prevents downstream signal transduction triggered by an acetyl-lysine. Alternatively, an antibody that specifically binds the unacetylated target acetylation site reduces the acetylation at that site and thus reduces activation of the protein mediated by acetylation of that site. Similarly, an unacetylated peptide may compete with an endogenous acetylation site for same kinases, thereby preventing or reducing the acetylation of the endogenous target protein. Alternatively, a peptide comprising an acetylated novel lysine site of the invention but lacking the ability to trigger signal transduction may competitively inhibit interaction of the endogenous protein with the same down-stream ligand(s).

The antibodies of the invention may also be used to target cancer cells for effector-mediated cell death. The antibody disclosed herein may be administered as a fusion molecule that includes a acetylation site-targeting portion joined to a cytotoxic moiety to directly kill cancer cells. Alternatively, the antibody may directly kill the cancer cells through complement-mediated or antibody-dependent cellular cytotoxicity.

Accordingly in one embodiment, the antibodies of the present disclosure may be used to deliver a variety of cytotoxic compounds. Any cytotoxic compound can be fused to the present antibodies. The fusion can be achieved chemically or genetically (e.g., via expression as a single, fused molecule). The cytotoxic compound can be a biological, such as a polypeptide, or a small molecule. As those skilled in the art will appreciate, for small molecules, chemical fusion is used, while for biological compounds, either chemical or genetic fusion can be used.

Non-limiting examples of cytotoxic compounds include therapeutic drugs, radiotherapeutic agents, ribosome-inactivating proteins (RIPs), chemotherapeutic agents, toxic peptides, toxic proteins, and mixtures thereof. The cytotoxic drugs can be intracellularly acting cytotoxic drugs, such as short-range radiation emitters, including, for example, short-range, high-energy α-emitters. Enzymatically active toxins and fragments thereof, including ribosome-inactivating proteins, are exemplified by saporin, luffin, momordins, ricin, trichosanthin, gelonin, abrin, etc. Procedures for preparing enzymatically active polypeptides of the immunotoxins are described in WO84/03508 and WO85/03508, which are hereby incorporated by reference. Certain cytotoxic moieties are derived from adriamycin, chlorambucil, daunomycin, methotrexate, neocarzinostatin, and platinum, for example.

Exemplary chemotherapeutic agents that may be attached to an antibody or antigen-binding fragment thereof include taxol, doxorubicin, verapamil, podophyllotoxin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, transplatinum, 5-fluorouracil, vincristin, vinblastin, or methotrexate.

Procedures for conjugating the antibodies with the cytotoxic agents have been previously described and are within the purview of one skilled in the art.

Alternatively, the antibody can be coupled to high energy radiation emitters, for example, a radioisotope, such as ¹³¹I, a γ-emitter, which, when localized at the tumor site, results in a killing of several cell diameters. See, e.g., S. E. Order, “Analysis, Results, and Future Prospective of the Therapeutic Use of Radiolabeled Antibody in Cancer Therapy”, Monoclonal Antibodies for Cancer Detection and Therapy, Baldwin et al. (eds.), pp. 303-316 (Academic Press 1985), which is hereby incorporated by reference. Other suitable radioisotopes include a-emitters, such as ²¹²Bi, ²¹³Bi, and ²¹¹At, and β-emitters, such as ¹⁸⁶Re and ⁹⁰Y.

Because many of the signaling proteins in which novel lysine acetylation sites of the invention occur also are expressed in normal cells and tissues, it may also be advantageous to administer a acetylation site-specific antibody with a constant region modified to reduce or eliminate ADCC or CDC to limit damage to normal cells. For example, effector function of antibodies may be reduced or eliminated by utilizing an IgG1 constant domain instead of an IgG2/4 fusion domain. Other ways of eliminating effector function can be envisioned such as, e.g., mutation of the sites known to interact with FcR or insertion of a peptide in the hinge region, thereby eliminating critical sites required for FcR interaction. Variant antibodies with reduced or no effector function also include variants as described previously herein.

The peptides and antibodies of the invention may be used in combination with other therapies or with other agents. Other agents include but are not limited to polypeptides, small molecules, chemicals, metals, organometallic compounds, inorganic compounds, nucleic acid molecules, oligonucleotides, aptamers, spiegelmers, antisense nucleic acids, locked nucleic acid (LNA) inhibitors, peptide nucleic acid (PNA) inhibitors, immunomodulatory agents, antigen-binding fragments, prodrugs, and peptidomimetic compounds. In certain embodiments, the antibodies and peptides of the invention may be used in combination with cancer therapies known to one of skill in the art.

In certain aspects, the present disclosure relates to combination treatments comprising a acetylation site-specific antibody described herein and immunomodulatory compounds, vaccines or chemotherapy. Illustrative examples of suitable immunomodulatory agents that may be used in such combination therapies include agents that block negative regulation of T cells or antigen presenting cells (e.g., anti-CTLA4 antibodies, anti-PD-L1 antibodies, anti-PDL-2 antibodies, anti-PD-1 antibodies and the like) or agents that enhance positive co-stimulation of T cells (e.g., anti-CD40 antibodies or anti 4-1BB antibodies) or agents that increase NK cell number or T-cell activity (e.g., inhibitors such as IMiDs, thalidomide, or thalidomide analogs). Furthermore, immunomodulatory therapy could include cancer vaccines such as dendritic cells loaded with tumor cells, proteins, peptides, RNA, or DNA derived from such cells, patient derived heat-shock proteins (hsp's) or general adjuvants stimulating the immune system at various levels such as CpG, Luivac®, Biostim®, Ribomunyl®, Imudon®, Bronchovaxom® or any other compound or other adjuvant activating receptors of the innate immune system (e.g., toll like receptor agonist, anti-CTLA-4 antibodies, etc.). Also, immunomodulatory therapy could include treatment with cytokines such as IL-2, GM-CSF and IFN-gamma.

Furthermore, combination of antibody therapy with chemotherapeutics could be particularly useful to reduce overall tumor burden, to limit angiogenesis, to enhance tumor accessibility, to enhance susceptibility to ADCC, to result in increased immune function by providing more tumor antigen, or to increase the expression of the T cell attractant LIGHT.

Pharmaceutical compounds that may be used for combinatory anti-tumor therapy include, merely to illustrate: aminoglutethimide, amsacrine, anastrozole, asparaginase, bcg, bicalutamide, bleomycin, buserelin, busulfan, camptothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic anti-tumor compounds may be categorized by their mechanism of action into groups, including, for example, the following classes of agents: anti-metabolites/anti-cancer agents, such as pyrimidine analogs (5-fluorouracil, floxuridine, capecitabine, gemcitabine and cytarabine) and purine analogs, folate inhibitors and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristine, vinblastine, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, mechlorethamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes—dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); immunomodulatory agents (thalidomide and analogs thereof such as lenalidomide (Revlimid, CC-5013) and CC-4047 (Actimid)), cyclophosphamide; anti-angiogenic compounds (TNP-470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylprednisolone, prednisone, and prenisolone); growth factor signal transduction kinase inhibitors; mitochondrial dysfunction inducers and caspase activators; and chromatin disruptors.

In certain embodiments, pharmaceutical compounds that may be used for combinatory anti-angiogenesis therapy include: (1) inhibitors of release of “angiogenic molecules,” such as bFGF (basic fibroblast growth factor); (2) neutralizers of angiogenic molecules, such as anti-βbFGF antibodies; and (3) inhibitors of endothelial cell response to angiogenic stimuli, including collagenase inhibitor, basement membrane turnover inhibitors, angiostatic steroids, fungal-derived angiogenesis inhibitors, platelet factor 4, thrombospondin, arthritis drugs such as D-penicillamine and gold thiomalate, vitamin D₃ analogs, alpha-interferon, and the like. For additional proposed inhibitors of angiogenesis, see Blood et al., Biochim. Biophys. Acta, 1032:89-118 (1990), Moses et al., Science, 248:1408-1410 (1990), Ingber et al., Lab. Invest., 59:44-51 (1988), and U.S. Pat. Nos. 5,092,885, 5,112,946, 5,192,744, 5,202,352, and 6,573,256. In addition, there are a wide variety of compounds that can be used to inhibit angiogenesis, for example, peptides or agents that block the VEGF-mediated angiogenesis pathway, endostatin protein or derivatives, lysine binding fragments of angiostatin, melanin or melanin-promoting compounds, plasminogen fragments (e.g., Kringles 1-3 of plasminogen), troponin subunits, inhibitors of vitronectin αᵥβ3, peptides derived from Saposin B, antibiotics or analogs (e.g., tetracycline or neomycin), dienogest-containing compositions, compounds comprising a MetAP-2 inhibitory core coupled to a peptide, the compound EM-138, chalcone and its analogs, and naaladase inhibitors. See, for example, U.S. Pat. Nos. 6,395,718, 6,462,075, 6,465,431, 6,475,784, 6,482,802, 6,482,810, 6,500,431, 6,500,924, 6,518,298, 6,521,439, 6,525,019, 6,538,103, 6,544,758, 6,544,947, 6,548,477, 6,559,126, and 6,569,845.

### 7. Diagnostic Uses

In a further aspect, the invention provides methods for detecting and quantitating phosphoyrlation at a novel lysine acetylation site of the invention. For example, peptides, including AQUA peptides of the invention, and antibodies of the invention are useful in diagnostic and prognostic evaluation of cancer, wherein the particular cancer is associated with the acetylation state of a novel acetylation site in Table 1, whether acetylated or deacetylated.

Methods of diagnosis can be performed in vitro using a biological sample (e.g., blood sample, lymph node biopsy or tissue) from a subject, or in vivo. The acetylation state or level at the lysine residue identified in the corresponding row in Column D of Table 1 may be assessed. A change in the acetylation state or level at the acetylation site, as compared to a control, indicates that the subject is suffering from, or susceptible to a for of cancer; for example, carcinoma.

In one embodiment, the acetylation state or level at a novel acetylation site is determined by an AQUA peptide comprising the acetylation site. The AQUA peptide may be acetylated or unacetylated at the specified lysine position.

In another embodiment, the acetylation state or level at a acetylation site is determined by an antibody or antigen-binding fragment thereof, wherein the antibody specifically binds the acetylation site. The antibody may be one that only binds to the acetylation site when the lysine residue is acetylated, but does not bind to the same sequence when the lysine is not acetylated; or vice versa.

In particular embodiments, the antibodies of the present application are attached to labeling moieties, such as a detectable marker. One or more detectable labels can be attached to the antibodies. Exemplary labeling moieties include radiopaque dyes, radiocontrast agents, fluorescent molecules, spin-labeled molecules, enzymes, or other labeling moieties of diagnostic value, particularly in radiologic or magnetic resonance imaging techniques.

A radiolabeled antibody in accordance with this disclosure can be used for in vitro diagnostic tests. The specific activity of an antibody, binding portion thereof, probe, or ligand, depends upon the half-life, the isotopic purity of the radioactive label, and how the label is incorporated into the biological agent. In immunoassay tests, the higher the specific activity, in general, the better the sensitivity. Radioisotopes useful as labels, e.g., for use in diagnostics, include iodine (¹³¹I or ¹²⁵I), indium (¹¹¹In), technetium (⁹⁹Tc), phosphorus (³²P), carbon (¹⁴C), and tritium (³H), or one of the therapeutic isotopes listed above.

Fluorophore and chromophore labeled biological agents can be prepared from standard moieties known in the art. Since antibodies and other proteins absorb light having wavelengths up to about 310 nm, the fluorescent moieties may be selected to have substantial absorption at wavelengths above 310 nm, such as for example, above 400 nm. A variety of suitable fluorescers and chromophores are described by Stryer, Science, 162:526 (1968) and Brand et al., Annual Review of Biochemistry, 41:843-868 (1972), which are hereby incorporated by reference. The antibodies can be labeled with fluorescent chromophore groups by conventional procedures such as those disclosed in U.S. Pat. Nos. 3,940,475, 4,289,747, and 4,376,110, which are hereby incorporated by reference.

The control may be parallel samples providing a basis for comparison, for example, biological samples drawn from a healthy subject, or biological samples drawn from healthy tissues of the same subject. Alternatively, the control may be a pre-determined reference or threshold amount. If the subject is being treated with a therapeutic agent, and the progress of the treatment is monitored by detecting the lysine acetylation state level at an acetylation site of the invention, a control may be derived from biological samples drawn from the subject prior to, or during the course of the treatment.

In certain embodiments, antibody conjugates for diagnostic use in the present application are intended for use in vitro, where the antibody is linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. In certain embodiments, secondary binding ligands are biotin and avidin or streptavidin compounds.

Antibodies of the invention may also be optimized for use in a flow cytometry (FC) assay to determine the activation/acetylation status of a target signaling protein in subjects before, during, and after treatment with a therapeutic agent targeted at inhibiting lysine acetylation at the acetylation site disclosed herein. For example, bone marrow cells or peripheral blood cells from patients may be analyzed by flow cytometry for target signaling protein acetylation, as well as for markers identifying various hematopoietic cell types. In this manner, activation status of the malignant cells may be specifically characterized. Flow cytometry may be carried out according to standard methods. See, e.g., Chow et al., Cytometry (Communications in Clinical Cytometry) 46: 72-78 (2001).

Alternatively, antibodies of the invention may be used in immunohistochemical (IHC) staining to detect differences in signal transduction or protein activity using normal and diseased tissues. IHC may be carried out according to well-known techniques. See, e.g., Antibodies: A Laboratory Manual, supra.

Peptides and antibodies of the invention may be also be optimized for use in other clinically-suitable applications, for example bead-based multiplex-type assays, such as IGEN, Luminex™ and/or Bioplex™ assay formats, or otherwise optimized for antibody arrays formats, such as reversed-phase array applications (see, e.g. Paweletz et al., Oncogene 20(16): 1981-89 (2001)). Accordingly, in another embodiment, the invention provides a method for the multiplex detection of the acetylation state or level at two or more acetylation sites of the invention (Table 1) in a biological sample, the method comprising utilizing two or more antibodies or AQUA peptides of the invention. In one preferred embodiment, two to five antibodies or AQUA peptides of the invention are used. In another preferred embodiment, six to ten antibodies or AQUA peptides of the invention are used, while in another preferred embodiment eleven to twenty antibodies or AQUA peptides of the invention are used.

In certain embodiments the diagnostic methods of the application may be used in combination with other cancer diagnostic tests.

The biological sample analyzed may be any sample that is suspected of having abnormal lysine acetylation at a novel acetylation site of the invention, such as a homogenized neoplastic tissue sample.

### 8. Screening Assays

In another aspect, the invention provides a method for identifying an agent that modulates lysine acetylation at a novel acetylation site of the invention, comprising: a) contacting a candidate agent with a peptide or protein comprising a novel acetylation site of the invention; and b) determining the acetylation state or level at the novel acetylation site. A change in the acetylation level of the specified lysine in the presence of the test agent, as compared to a control, indicates that the candidate agent potentially modulates lysine acetylation at a novel acetylation site of the invention.)

In one embodiment, the acetylation state or level at a novel acetylation site is determined by an AQUA peptide comprising the acetylation site. The AQUA peptide may be acetylated or unacetylated at the specified lysine position.

In another embodiment, the acetylation state or level at a acetylation site is determined by an antibody or antigen-binding fragment thereof, wherein the antibody specifically binds the acetylation site. The antibody may be one that only binds to the acetylation site when the lysine residue is acetylated, but does not bind to the same sequence when the lysine is not acetylated; or vice versa.

In particular embodiments, the antibodies of the present application are attached to labeling moieties, such as a detectable marker.

The control may be parallel samples providing a basis for comparison, for example, the acetylation level of the target protein or peptide in absence of the testing agent. Alternatively, the control may be a pre-determined reference or threshold amount.

### 9. Immunoassays

In another aspect, the present application concerns immunoassays for binding, purifying, quantifying and otherwise generally detecting the acetylation state or level at a novel acetylation site of the invention.

Assays may be homogeneous assays or heterogeneous assays. In a homogeneous assay the immunological reaction usually involves a acetylation site-specific antibody of the invention, a labeled analyte, and the sample of interest. The signal arising from the label is modified, directly or indirectly, upon the binding of the antibody to the labeled analyte. Both the immunological reaction and detection of the extent thereof are carried out in a homogeneous solution. Immunochemical labels that may be used include free radicals, radioisotopes, fluorescent dyes, enzymes, bacteriophages, coenzymes, and so forth.

In a heterogeneous assay approach, the reagents are usually the specimen, a acetylation site-specific antibody of the invention, and suitable means for producing a detectable signal. Similar specimens as described above may be used. The antibody is generally immobilized on a support, such as a bead, plate or slide, and contacted with the specimen suspected of containing the antigen in a liquid phase. The support is then separated from the liquid phase and either the support phase or the liquid phase is examined for a detectable signal using means for producing such signal. The signal is related to the presence of the analyte in the specimen. Means for producing a detectable signal include the use of radioactive labels, fluorescent labels, enzyme labels, and so forth.

Acetylation site-specific antibodies disclosed herein may be conjugated to a solid support suitable for a diagnostic assay (e.g., beads, plates, slides or wells formed from materials such as latex or polystyrene) in accordance with known techniques, such as precipitation.

In certain embodiments, immunoassays are the various types of enzyme linked immunoadsorbent assays (ELISAs) and radioimmunoassays (RIA) known in the art. Immunohistochemical detection using tissue sections is also particularly useful. However, it will be readily appreciated that detection is not limited to such techniques, and Western blotting, dot and slot blotting, FACS analyses, and the like may also be used. The steps of various useful immunoassays have been described in the scientific literature, such as, e.g., Nakamura et al., in Enzyme Immunoassays: Heterogeneous and Homogeneous Systems, Chapter 27 (1987), incorporated herein by reference.

In general, the detection of immunocomplex formation is well known in the art and may be achieved through the application of numerous approaches. These methods are based upon the detection of radioactive, fluorescent, biological or enzymatic tags. Of course, one may find additional advantages through the use of a secondary binding ligand such as a second antibody or a biotin/avidin ligand binding arrangement, as is known in the art.

The antibody used in the detection may itself be conjugated to a detectable label, wherein one would then simply detect this label. The amount of the primary immune complexes in the composition would, thereby, be determined.

Alternatively, the first antibody that becomes bound within the primary immune complexes may be detected by means of a second binding ligand that has binding affinity for the antibody. In these cases, the second binding ligand may.be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are washed extensively to remove any non-specifically bound labeled secondary antibodies or ligands, and the remaining label in the secondary immune complex is detected.

An enzyme linked immunoadsorbent assay (ELISA) is a type of binding assay. In one type of ELISA, acetylation site-specific antibodies disclosed herein are immobilized onto a selected surface exhibiting protein affinity, such as a well in a polystyrene microtiter plate. Then, a suspected neoplastic tissue sample is added to the wells. After binding and washing to remove non-specifically bound immune complexes, the bound target signaling protein may be detected.

In another type of ELISA, the neoplastic tissue samples are immobilized onto the well surface and then contacted with the acetylation site-specific antibodies disclosed herein. After binding and washing to remove non-specifically bound immune complexes, the bound acetylation site-specific antibodies are detected.

Irrespective of the format used, ELISAs have certain features in common, such as coating, incubating or binding, washing to remove non-specifically bound species, and detecting the bound immune complexes.

The radioimmunoassay (RIA) is an analytical technique which depends on the competition (affinity) of an antigen for antigen-binding sites on antibody molecules. Standard curves are constructed from data gathered from a series of samples each containing the same known concentration of labeled antigen, and various, but known, concentrations of unlabeled antigen. Antigens are labeled with a radioactive isotope tracer. The mixture is incubated in contact with an antibody. Then the free antigen is separated from the antibody and the antigen bound thereto. Then, by use of a suitable detector, such as a gamma or beta radiation detector, the percent of either the bound or free labeled antigen or both is determined. This procedure is repeated for a number of samples containing various known concentrations of unlabeled antigens and the results are plotted as a standard graph. The percent of bound tracer antigens is plotted as a function of the antigen concentration. Typically, as the total antigen concentration increases the relative amount of the tracer antigen bound to the antibody decreases. After the standard graph is prepared, it is thereafter used to determine the concentration of antigen in samples undergoing analysis.

In an analysis, the sample in which the concentration of antigen is to be determined is mixed with a known amount of tracer antigen. Tracer antigen is the same antigen known to be in the sample but which has been labeled with a suitable radioactive isotope. The sample with tracer is then incubated in contact with the antibody. Then it can be counted in a suitable detector which counts the free antigen remaining in the sample. The antigen bound to the antibody or immunoadsorbent may also be similarly counted. Then, from the standard curve, the concentration of antigen in the original sample is determined.

### 10. Pharmaceutical Formulations and Methods of Administration

Methods of administration of therapeutic agents, particularly peptide and antibody therapeutics, are well-known to those of skill in the art.

Peptides of the invention can be administered in the same manner as conventional peptide type pharmaceuticals. Preferably, peptides are administered parenterally, for example, intravenously, intramuscularly, intraperitoneally, or subcutaneously. When administered orally, peptides may be proteolytically hydrolyzed. Therefore, oral application may not be usually effective. However, peptides can be administered orally as a formulation wherein peptides are not easily hydrolyzed in a digestive tract, such as liposome-microcapsules. Peptides may be also administered in suppositories, sublingual tablets, or intranasal spray.

If administered parenterally, a preferred pharmaceutical composition is an aqueous solution that, in addition to a peptide of the invention as an active ingredient, may contain for example, buffers such as phosphate, acetate, etc., osmotic pressure-adjusting agents such as sodium chloride, sucrose, and sorbitol, etc., antioxidative or antioxygenic agents, such as ascorbic acid or tocopherol and preservatives, such as antibiotics. The parenterally administered composition also may be a solution readily usable or in a lyophilized form which is dissolved in sterile water before administration.

The pharmaceutical formulations, dosage forms, and uses described below generally apply to antibody-based therapeutic agents, but are also useful and can be modified, where necessary, for making and using therapeutic agents of the disclosure that are not antibodies.

To achieve the desired therapeutic effect, the acetylation site-specific antibodies or antigen-binding fragments thereof can be administered in a variety of unit dosage forms. The dose will vary according to the particular antibody. For example, different antibodies may have different masses and/or affinities, and thus require different dosage levels. Antibodies prepared as Fab or other fragments will also require differing dosages than the equivalent intact immunoglobulins, as they are of considerably smaller mass than intact immunoglobulins, and thus require lower dosages to reach the same molar levels in the patient's blood. The dose will also vary depending on the manner of administration, the particular symptoms of the patient being treated, the overall health, condition, size, and age of the patient, and the judgment of the prescribing physician. Dosage levels of the antibodies for human subjects are generally between about 1 mg per kg and about 100 mg per kg per patient per treatment, such as for example, between about 5 mg per kg and about 50 mg per kg per patient per treatment. In terms of plasma concentrations, the antibody concentrations may be in the range from about 25 μg/mL to about 500 μg/mL. However, greater amounts may be required for extreme cases and smaller amounts may be sufficient for milder cases.

Administration of an antibody will generally be performed by a parenteral route, typically via injection such as intra-articular or intravascular injection (e.g., intravenous infusion) or intramuscular injection. Other routes of administration, e.g., oral (p.o.), may be used if desired and practicable for the particular antibody to be administered. An antibody can also be administered in a variety of unit dosage forms and their dosages will also vary with the size, potency, and in vivo half-life of the particular antibody being administered. Doses of a acetylation site-specific antibody will also vary depending on the manner of administration, the particular symptoms of the patient being treated, the overall health, condition, size, and age of the patient, and the judgment of the prescribing physician.

The frequency of administration may also be adjusted according to various parameters. These include the clinical response, the plasma half-life of the antibody, and the levels of the antibody in a body fluid, such as, blood, plasma, serum, or synovial fluid. To guide adjustment of the frequency of administration, levels of the antibody in the body fluid may be monitored during the course of treatment.

Formulations particularly useful for antibody-based therapeutic agents are also described in U.S. Patent App. Publication Nos. 20030202972, 20040091490 and 20050158316. In certain embodiments, the liquid formulations of the application are substantially free of surfactant and/or inorganic salts. In another specific embodiment, the liquid formulations have a pH ranging from about 5.0 to about 7.0. In yet another specific embodiment, the liquid formulations comprise histidine at a concentration ranging from about 1 mM to about 100 mM. In still another specific embodiment, the liquid formulations comprise histidine at a concentration ranging from 1 mM to 100 mM. It is also contemplated that the liquid formulations may further comprise one or more excipients such as a saccharide, an amino acid (e.g., arginine, lysine, and methionine) and a polyol. Additional descriptions and methods of preparing and analyzing liquid formulations can be found, for example, in PCT publications WO 03/106644, WO 04/066957, and WO 04/091658.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the pharmaceutical compositions of the application.

In certain embodiments, formulations of the subject antibodies are pyrogen-free formulations which are substantially free of endotoxins and/or related pyrogenic substances. Endotoxins include toxins that are confined inside microorganisms and are released when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, it is advantageous to remove even low amounts of endotoxins from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in amounts of several hundred or thousand milligrams per kilogram body weight, as can be the case with monoclonal antibodies, it is advantageous to remove even trace amounts of endotoxin.

The amount of the formulation which will be therapeutically effective can be determined by standard clinical techniques. In addition, in vitro assays may optionally be used to help identify optimal dosage ranges. The precise dose to be used in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems. The dosage of the compositions to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms. For example, the actual patient body weight may be used to calculate the dose of the formulations in milliliters (mL) to be administered. There may be no downward adjustment to “ideal” weight. In such a situation, an appropriate dose may be calculated by the following formula:

Dose (mL)=[patient weight (kg)×dose level (mg/kg)/drug concentration (mg/mL)]

For the purpose of treatment of disease, the appropriate dosage of the compounds (for example, antibodies) will depend on the severity and course of disease, the patient's clinical history and response, the toxicity of the antibodies, and the discretion of the attending physician. The initial candidate dosage may be administered to a patient. The proper dosage and treatment regimen can be established by monitoring the progress of therapy using conventional techniques known to those of skill in the art.

The formulations of the application can be distributed as articles of manufacture comprising packaging material and a pharmaceutical agent which comprises, e.g., the antibody and a pharmaceutically acceptable carrier as appropriate to the mode of administration. The packaging material will include a label which indicates that the formulation is for use in the treatment of prostate cancer.

### 11. Kits

Antibodies and peptides (including AQUA peptides) of the invention may also be used within a kit for detecting the acetylation state or level at a novel acetylation site of the invention, comprising at least one ofthe following: an AQUA peptide comprising the acetylation site, or an antibody or an antigen-binding fragment thereof that binds to an amino acid sequence comprising the acetylation site. Such a kit may further comprise a packaged combination of reagents in predetermined amounts with instructions for performing the diagnostic assay. Where the antibody is labeled with an enzyme, the kit will include substrates and co-factors required by the enzyme. In addition, other additives may be included such as stabilizers, buffers and the like. The relative amounts of the various reagents may be varied widely to provide for concentrations in solution of the reagents that substantially optimize the sensitivity of the assay. Particularly, the reagents may be provided as dry powders, usually lyophilized, including excipients that, on dissolution, will provide a reagent solution having the appropriate concentration.

The following Examples are provided only to further illustrate the invention, and are not intended to limit its scope, except as provided in the claims appended hereto. The invention encompasses modifications and variations of the methods taught herein which would be obvious to one of ordinary skill in the art.

### Example 1

### Isolation of Acetyl-Lysine Containing Peptides from Extracts of Cancer Cell Lines and Identification of Novel Acetylation Sites

In order to discover novel lysine acetylation sites in cancer (including carcinoma), IAP isolation techniques were used to identify acetyl-lysine containing peptides in cell extracts from human cancer cell lines and patient cell lines identified in Column G of Table 1 including H23, H3255, H520, HCC78, HCC827, HCT116, HCT15, HCT8, HEP-G2, HeLa, K562, NB-4, NCI-H716, OCI/AML2, OCI/AML3, SIL-ALL, SNU-C2B, SW620, sw48, sw480.

Tryptic acetyl-lysine containing peptides were purified and analyzed from extracts of each of the cell lines mentioned above, as follows. Cells were cultured in DMEM medium or RPMI 1640 medium supplemented with 10% fetal bovine serum and penicillin/streptomycin.

Suspension cells were harvested by low speed centrifugation. After complete aspiration of medium, cells were resuspended in 1 mL lysis buffer per 1.25×10⁸ cells (20 mM HEPES pH 8.0, 9 M urea, 1 mM sodium vanadate, supplemented or not with 2.5 mM sodium pyro-phosphate, 1 mM β-glycerol-phosphate) and sonicated.

Adherent cells at about 80% confluency were starved in medium without serum overnight and stimulated, with ligand depending on the cell type or not stimulated. After complete aspiration of medium from the plates, cells were scraped off the plate in 10 ml lysis buffer per 2×10⁸ cells (20 mM HEPES pH 8.0, 9 M urea, 1 mM sodium vanadate, supplemented with 2.5 mM sodium pyrophosphate, 1 mM β-glycerol-phosphate) and sonicated.

Frozen tissue samples were cut to small pieces, homogenize in lysis buffer (20 mM HEPES pH 8.0, 9 M Urea, 1 mN sodium vanadate, supplemented with 2.5 mM sodium pyrophosphate, 1 mM b-glycerol-phosphate, 1 ml lysis buffer for 100 mg of frozen tissue) using a polytron for 2 times of 20 sec. each time. Homogenate is then briefly sonicated.

Sonicated cell lysates were cleared by centrifugation at 20,000×g, and proteins were reduced with DTT at a final concentration of 4.1 mM and alkylated with iodoacetamide at 8.3 mM. For digestion with trypsin, protein extracts were diluted in 20 mM HEPES pH 8.0 to a final concentration of 2 M urea and soluble TLCK-trypsin (Worthington) was added at 10-20 μg/mL. Digestion was performed for 1-2 days at room temperature.

Trifluoroacetic acid (TFA) was added to protein digests to a final concentration of 1%, precipitate was removed by centrifugation, and digests were loaded onto Sep-Pak C₁₈ columns (Waters) equilibrated with 0.1% TFA. A column volume of 0.7-1.0 ml was used per 2×10⁸ cells. Columns were washed with 15 volumes of 0.1% TFA, followed by 4 volumes of 5% acetonitrile (MeCN) in 0.1% TFA. Peptide fraction I was obtained by eluting columns with 2 volumes each of 8, 12, and 15% MeCN in 0.1% TFA and combining the eluates. Fractions II and III were a combination of eluates after eluting columns with 18, 22, 25% MeCN in 0.1% TFA and with 30, 35, 40% MeCN in 0.1% TFA, respectively. All peptide fractions were lyophilized.

Peptides from each fraction corresponding to 2×10⁸ cells were dissolved in 1 ml of IAP buffer (20 mM Tris/HCl or 50 mM MOPS pH 7.2, 10 mM sodium phosphate, 50 mM NaCl) and insoluble matter (mainly in peptide fractions III) was removed by centrifugation. IAP was performed on each peptide fraction separately. The acetyl-lysine monoclonal antibody (Cell Signaling Technology, Inc., catalog number 8691) was coupled at 4 mg/ml beads to protein G (Roche), respectively. Immobilized antibody (15 μl, 60 μg) was added as 1:1 slurry in IAP buffer to 1 ml of each peptide fraction, and the mixture was incubated overnight at 4° C. with gentle rotation. The immobilized antibody beads were washed three times with 1 ml IAP buffer and twice with 1 ml water, all at 4° C. Peptides were eluted from beads by incubation with 75 μl of 0.1% TFA at room temperature for 10 minutes.

Alternatively, one single peptide fraction was obtained from Sep-Pak C18 columns by elution with 2 volumes each of 10%, 15%, 20%, 25%, 30%, 35% and 40% acetonitirile in 0.1% TFA and combination of all eluates. IAP on this peptide fraction was performed as follows: After

lyophilization, peptide was dissolved in 1.4 ml IAP buffer (MOPS pH 7.2,

10 mM sodium phosphate, 50 mM NaCl) and insoluble matter was removed by centrifugation. Immobilized antibody (40 μl, 160 μg) was added as 1:1 slurry in IAP buffer, and the mixture was incubated overnight at 4° C. with gentle shaking. The immobilized antibody beads were washed three times with 1 ml IAP buffer and twice with 1 ml water, all at 4° C. Peptides were eluted from beads by incubation with 55 μl of 0.15% TFA at room temperature for 10 min (eluate 1), followed by a wash of the beads (eluate 2) with 45 μl of 0.15% TEA. Both eluates were combined.

### Analysis by LC-MS/MS Mass Spectrometry.

40 μl or more of IAP eluate were purified by 0.2 μl StageTips or ZipTips. Peptides were eluted from the microcolumns with 1 μl of 40% MeCN, 0.1% TFA (fractions I and II) or 1 μl of 60% MeCN, 0.1% TFA (fraction III) into 7.6-9.0 μl of 0.4% acetic acid/0.005% heptafluorobutyric acid. For single fraction analysis, 1 μl of 60% MeCN, 0.1% TFA, was used for elution from the microcolumns. This sample was loaded onto a 10 cm×75 μm PicoFrit capillary column (New Objective) packed with Magic C18 AQ reversed-phase resin (Michrom Bioresources) using a Famos autosampler with an inert sample injection valve (Dionex). The column was then developed with a 45-min linear gradient of acetonitrile delivered at 200 nl/min (Ultimate, Dionex), and tandem mass spectra were collected in a data-dependent manner with an LTQ ion trap mass spectrometer essentially as described by Gygi et al., supra.

### Database Analysis & Assignments.

MS/MS spectra were evaluated using TurboSequest in the Sequest Browser package (v. 27, rev. 12) supplied as part of Bio Works 3.0 (ThermoFinnigan). Individual MS/MS spectra were extracted from the raw data file using the Sequest Browser program CreateDta, with the following settings: bottom MW, 700; top MW, 4,500; minimum number of ions, 20 (40 for LTQ); minimum TIC, 4×10⁵ (2×10³ for LTQ); and precursor charge state, unspecified. Spectra were extracted from the beginning of the raw data file before sample injection to the end of the eluting gradient. The IonQuest and VuDta programs were not used to further select MS/MS spectra for Sequest analysis. MS/MS spectra were evaluated with the following TurboSequest parameters: peptide mass tolerance, 2.5; fragment ion tolerance, 0.0 (1.0 for LTQ); maximum number of differential amino acids per modification, 4; mass type parent, average; mass type fragment, average; maximum number of internal cleavage sites, 10; neutral losses of water and ammonia from b and y ions were considered in the correlation analysis. Proteolytic enzyme was specified except for spectra collected from elastase digests.

Searches were performed against the then current NCBI human protein database. Peptides that did not match RefSeq were compared to the then current NCBI GenPept database. Cysteine carboxamidomethylation was specified as a static modification, and acetylation was allowed as a variable modification on lysine residues alone. It was determined that restricting acetylation to lysine residues had little effect on the number of acetylation sites assigned.

In proteomics research, it is desirable to validate protein identifications based solely on the observation of a single peptide in one experimental result, in order to indicate that the protein is, in fact, present in a sample. This has led to the development of statistical methods for validating peptide assignments, which are not yet universally accepted, and guidelines for the publication of protein and peptide identification results (see Carr et al., Mol. Cell Proteomics 3: 531-533 (2004)), which were followed in this Example. However, because the immunoaffinity strategy separates acetylated peptides from unacetylated peptides, observing just one phosphopeptide from a protein is a common result, since many acetylated proteins have only one lysine-acetylated site. For this reason, it is appropriate to use additional criteria to validate phosphopeptide assignments. Assignments are likely to be correct if any of these additional criteria are met: (i) the same phosphopeptide sequence is assigned to co-eluting ions with different charge states, since the MS/MS spectrum changes markedly with charge state; (ii) the acetylation site is found in more than one peptide sequence context due to sequence overlaps from incomplete proteolysis or use of proteases other than trypsin; (iii) the acetylation site is found in more than one peptide sequence context due to homologous but not identical protein isoforms; (iv) the acetylation site is found in more than one peptide sequence context due to homologous but not identical proteins among species; and (v) acetylation sites validated by MS/MS analysis of synthetic phosphopeptides corresponding to assigned sequences, since the ion trap mass spectrometer produces highly reproducible MS/MS spectra. The last criterion is routinely used to confirm novel site assignments of particular interest.

All spectra and all sequence assignments made by Sequest were imported into a relational database. The following Sequest scoring thresholds were used to select phosphopeptide assignments that are likely to be correct: RSp<6, XCorr≧2.2, and DeltaCN>0.099. Further, the sequence assignments could be accepted or rejected with respect to accuracy by using the following conservative, two-step process.

In the first step, a subset of high-scoring sequence assignments should be selected by filtering for XCorr values of at least 1.5 for a charge state of +1, 2.2 for +2, and 3.3 for +3, allowing a maximum RSp value of 10. Assignments in this subset should be rejected if any of the following criteria are satisfied: (i) the spectrum contains at least one major peak (at least 10% as intense as the most intense ion in the spectrum) that can not be mapped to the assigned sequence as an a, b, or y ion, as an ion arising from neutral-loss of water or ammonia from a b or y ion, or as a multiply protonated ion; (ii) the spectrum does not contain a series of b or y ions equivalent to at least six uninterrupted residues; or (iii) the sequence is not observed at least five times in all the studies conducted (except for overlapping sequences due to incomplete proteolysis or use of proteases other than trypsin).

In the second step, assignments with below-threshold scores should be accepted if the low-scoring spectrum shows a high degree of similarity to a high-scoring spectrum collected in another study, which simulates a true reference library-searching strategy.

### Example 2

### Production of Acetylation Site-Specific Polyclonal Antibodies

Polyclonal antibodies that specifically bind a novel acetylation site of the invention (Table 1/FIG. 2) only when the lysine residue is acetylated (and does not bind to the same sequence when the lysine is not acetylated), and vice versa, are produced according to standard methods by first constructing a synthetic peptide antigen comprising the acetylation site and then immunizing an animal to raise antibodies against the antigen, as further described below. Production of exemplary polyclonal antibodies is provided below.

### A. MYST3 (Lysine 350).

An 11 amino acid acetyl-peptide antigen, QNTVSk*GPFSK

(SEQ NO: 28; k*=acetyl-lysine), which comprises the acetylation site derived from human MYST3 (a chromatin or DNA binding/repair/replication protein), Lys 350 being the acetylatable residue), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) acetylation site-specific polyclonal antibodies as described in Immunization/Screening below.

### B. PPIL2 (Lysine 482).

A 13 amino acid acetyl-peptide antigen, QGVGk*YINPAATK (SEQ ID NO: 57; k*=acetyl-lysine), which comprises the acetylation site derived from human PPIL2 (an enzyme protein, Lys 482 being the acetylatable residue), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) acetylation site-specific polyclonal antibodies as described in Immunization/Screening below.

### C. SLC25A5 (Lysine 96).

A 14 amino acid acetyl-peptide antigen, DKYk*QIFLOGVDKR (SEQ ID NO: 80; k*=acetyl-lysine, which comprises the acetylation site derived from human SLC25A5 (a receptor/channel/transporter/cell surface protein, Lys 96 being the acetylatable residue), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) acetylation site-specific polyclonal antibodies as described in Immunization/Screening below.

### Immunization/Screening.

A synthetic acetyl-peptide antigen as described in A-C above is coupled to KLH, and rabbits are injected intradermally (ID) on the back with antigen in complete Freunds adjuvant (500 μg antigen per rabbit). The rabbits are boosted with same antigen in incomplete Freund adjuvant (250 μg antigen per rabbit) every three weeks. After the fifth boost, bleeds are collected. The sera are purified by Protein A-affinity chromatography by standard methods (see ANTIBODIES: A LABORATORY MANUAL, Cold Spring Harbor, supra.). The eluted immunoglobulins are further loaded onto an unacetylated synthetic peptide antigen-resin Knotes column to pull out antibodies that bind the unacetylated form of the acetylation sites. The flow through fraction is collected and applied onto an acetyl-synthetic peptide antigen—resin column to isolate antibodies that bind the acetylated form of the acetylation sites. After washing the column extensively, the bound antibodies (i.e. antibodies that bind the acetylated peptides described in A-C above, but do not bind the unacetylated form of the peptides) are eluted and kept in antibody storage buffer.

The isolated antibody is then tested for phospho-specificity using Western blot assay using an appropriate cell line that expresses (or overexpresses) target acetyl-protein (i.e. acetylated MYST3, PPIL2 and SLC25A5), for example, sw48 or collerectal carcinoma. Cells are cultured in DMEM or RPMI supplemented with 10% FCS. Cell are collected, washed with PBS and directly lysed in cell lysis buffer. The protein concentration of cell lysates is then measured. The loading buffer is added into cell lysate and the mixture is boiled at 100° C. for 5. minutes. 20 μl (10 μg protein) of sample is then added onto 7.5% SDS-PAGE gel.

A standard Western blot may be performed according to the Immunoblotting Protocol set out in the CELL SIGNALING TECHNOLOGY, INC. 2003-04 Catalogue, p. 390. The isolated acetylation site-specific antibody is used at dilution 1:1000. Phospho-specificity of the antibody will be shown by binding of only the acetylated form of the target amino acid sequence. Isolated acetylation site-specific polyclonal antibody does not (substantially) recognize the same target sequence when not acetylated at the specified lysine position (e.g., the antibody does not bind to SLC25A5 in the non-stimulated cells, when lysine 96 is not acetylated).

In order to confirm the specificity of the isolated antibody, different cell lysates containing various acetylated signaling proteins other than the target protein are prepared. The Western blot assay is performed again using these cell lysates. The acetylation site-specific polyclonal antibody isolated as described above is used (1:1000 dilution) to test reactivity with the different acetylated non-target proteins. The acetylation site-specific antibody does not significantly cross-react with other acetylated signaling proteins that do not have the described acetylation site, although occasionally slight binding to a highly homologous sequence on another protein may be observed. In such case the antibody may be further purified using affinity chromatography, or the specific immunoreactivity cloned by rabbit hybridoma technology.

### Example 3

### Production of Acetylation Site-specific Monoclonal Antibodies

Monoclonal antibodies that specifically bind a novel acetylation site of the invention (Table 1) only when the lysine residue is acetylated (and does not bind to the same sequence when the lysine is not acetylated) are produced according to standard methods by first constructing a synthetic peptide antigen comprising the acetylation site and then immunizing an animal to raise antibodies against the antigen, and harvesting spleen cells from such animals to produce fusion hybridomas, as further described below. Production of exemplary monoclonal antibodies is provided below.

### A. CaRHSP1 (Lysine 68).

An 8 amino acid acetyl-peptide antigen, GVCk*CFCR (SEQ ID NO: 82; k*=acetyl-lysine), which comprises the acetylation site derived from human CaRHSP1 (an RNA binding protein, Lys 68 being the phosphorylatable residue), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of acetylation site-specific monoclonal antibodies as described in Immunization/Fusion/Screening below.

### B. ELAC2 (Lysine152).

A 13 amino acid phospho-peptide antigen, YLEAIk*IFSGPLK (SEQ ID NO: 91; k*=acetyl-lysine), which comprises the acetylation site derived from human ELAC2 (an RNA binding protein, Lys 152 being the phosphorylatable residue), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of acetylation site-specific monoclonal antibodies as described in Immunization/Fusion/Screening below.

C. eIF3-eta (Lysine 729).

A 9 amino acid acetyl-peptide antigen, KYSk*IFEQK (SEQ ID NO: 149; k*=acetyl-lysine), which comprises the acetylation site derived from human eIF3-eta (a translational regulator protein, Lys 729 being the acetylatable residue), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of acetylation site-specific monoclonal antibodies as described in Immunization/Fusion/Screening below.

### Immunization/Fusion/Screening.

A synthetic acetyl-peptide antigen as described in A-C above is coupled to KLH, and BALB/C mice are injected intradermally (ID) on the back with antigen in complete Freunds adjuvant (e.g., 50 μg antigen per mouse). The mice are boosted with same antigen in incomplete Freund adjuvant (e.g. 25 μg antigen per mouse) every three weeks. After the fifth boost, the animals are sacrificed and spleens are harvested.

Harvested spleen cells are fused to SP2/0 mouse myeloma fusion partner cells according to the standard protocol of Kohler and Milstein (1975). Colonies originating from the fusion are screened by ELISA for reactivity to the acetyl-peptide and non-acetyl-peptide forms of the antigen and by Western blot analysis (as described in Example 1 above). Colonies found to be positive by ELISA to the acetyl-peptide while negative to the non-acetyl-peptide are further characterized by Western blot analysis. Colonies found to be positive by Western blot analysis are subcloned by limited dilution. Mouse ascites are produced from a single clone obtained from subcloning, and tested for acetyl-specificity (against the CaRHSP1, ELAC2 or eIF3-eta) acetyl-peptide antigen, as the case may be) on ELISA. Clones identified as positive on Western blot analysis using cell culture supernatant as having acetyl-specificity, as indicated by a strong band in the induced lane and a weak band in the uninduced lane of the blot, are isolated and subcloned as clones producing monoclonal antibodies with the desired specificity.

Ascites fluid from isolated clones may be further tested by Western blot analysis. The ascites fluid should produce similar results on Western blot analysis as observed previously with the cell culture supernatant, indicating acetyl-specificity against the acetylated target.

### Example 4

### Production and Use of AQUA Peptides for Detecting and Quantitating Acetylation at a Novel Acetylation Site

Heavy-isotope labeled peptides (AQUA peptides (internal standards)) for the detecting and quantitating a novel acetylation site of the invention (Table 1) only when the lysine residue is acetylated are produced according to the standard AQUA methodology (see Gygi et al., Gerber et al., supra.) methods by first constructing a synthetic peptide standard corresponding to the acetylation site sequence and incorporating a heavy-isotope label. Subsequently, the MSⁿ and LC-SRM signature of the peptide standard is validated, and the AQUA peptide is used to quantify native peptide in a biological sample, such as a digested cell extract. Production and use of exemplary AQUA peptides is provided below.

### A. RPL13A (Lysine 25).

An AQUA peptide comprising the sequence, LAAIVAk*QVLLGR (SEQ ID NO: 159; k*=acetyl-lysine; Valine being ¹⁴/¹⁵N-labeled, as indicated in bold), which comprises the acetylation site derived from RPL13A (Lys 25 being the acetylatable residue), is constructed according to standard synthesis techniques using, e.g., a Rainin/Protein Technologies, Inc., Symphony peptide synthesizer (see Merrifield, supra.) as further described below in Synthesis & MS/MS Signature. The RPL13A (Lys 25) AQUA peptide is then spiked into a biological sample to quantify the amount of acetylated RPL13A (Lys 25) in the sample, as further described below in Analysis & Quantification.

### B. RPL24 (Lysine 27).

An AQUA peptide comprising the sequence TDGk*VFQFLNAK ((SEQ ID NO: 162; k*=acetyl-lysine; Proline being ¹⁴C/¹⁵N-labeled, as indicated in bold), which comprises the acetylation site derived from human RPL24 (Lys 27 being the acetylatable residue), is constructed according to standard synthesis techniques using, e.g., a Rainin/Protein Technologies, Inc., Symphony peptide synthesizer (see Merrifield, supra.) as further described below in Synthesis & MS/MS Signature. The RPL24 (Lys 27) AQUA peptide is then spiked into a biological sample to quantify the amount of acetylated RPL24 (Lys 27) in the sample, as further described below in Analysis & Quantification.

### C. SAE2 (Lysine 253)

An AQUA peptide comprising the sequence STGYDPVk*LFTK (SEQ ID NO: 176; k*=acetyl-lysine; Leucine being ¹⁴C/¹⁵N-labeled, as indicated in bold), which comprises the acetylation site derived from human SAE 2 (Lys 253 being the acetylatable residue), is constructed according to standard synthesis techniques using, e.g., a Rainin/Protein Technologies, Inc., Symphony peptide synthesizer (see Merrifield, supra.) as further described below in Synthesis & MS/MS Signature. The SAE 2 (Lys 253) AQUA peptide is then spiked into a biological sample to quantify the amount of acetylated SAE 2 (Lys 253) in the sample, as further described below in Analysis & Quantification.

### D. UBE2S (Lysine 68).

An AQUA peptide comprising the sequence LLLKk*DFPASPPK ((SEQ ID NO: 179; k*=acetyl-lysine; proline being ¹⁴C/¹⁵N-labeled, as indicated in bold), which comprises the acetylation site derived from human UBE2S (Lys 68 being the acetylatable residue), is constructed according to standard synthesis techniques using, e.g., a Rainin/Protein Technologies, Inc., Symphony peptide synthesizer (see Merrifield, supra.) as further described below in Synthesis & MS/MS Signature. The UBE2S (Lys 68) AQUA peptide is then spiked into a biological sample to quantify the amount of acetylated UBE2S (Lys 68) in the sample, as further described below in Analysis & Quantification.

### Synthesis & MS/MS Spectra.

Fluorenylmethoxycarbonyl (Fmoc)-derivatized amino acid monomers may be obtained from AnaSpec (San Jose, Calif.). Fmoc-derivatized stable-isotope monomers containing one ¹⁵N and five to nine ¹³C atoms may be obtained from Cambridge Isotope Laboratories (Andover, Mass.). Preloaded Wang resins may be obtained from Applied Biosystems. Synthesis scales may vary from 5 to 25 μmol. Amino acids are activated in situ with 1-H-benzotriazolium, 1-bis(dimethylamino)methylene]-hexafluorophosphate (1-),3-oxide:1-hydroxybenzotriazole hydrate and coupled at a 5-fold molar excess over peptide. Each coupling cycle is followed by capping with acetic anhydride to avoid accumulation of one-residue deletion peptide by-products. After synthesis peptide-resins are treated with a standard scavenger-containing trifluoroacetic acid (TFA)-water cleavage solution, and the peptides are precipitated by addition to cold ether. Peptides (i.e. a desired AQUA peptide described in A-D above) are purified by reversed-phase C18 HPLC using standard TFA/acetonitrile gradients and characterized by matrix-assisted laser desorption ionization-time of flight (Biflex III, Bruker Daltonics, Billerica, Mass.) and ion-trap (ThermoFinnigan, LCQ DecaXP or LTQ) MS.

MS/MS spectra for each AQUA peptide should exhibit a strong y-type ion peak as the most intense fragment ion that is suitable for use in an SRM monitoring/analysis. Reverse-phase microcapillary columns (0.1 Å˜150-220 mm) are prepared according to standard methods. An Agilent 1100 liquid chromatograph may be used to develop and deliver a solvent gradient [0.4% acetic acid/0.005% heptafluorobutyric acid (HFBA)/7% methanol and 0.4% acetic acid/0.005% HFBA/65% methanol/35% acetonitrile] to the microcapillary column by means of a flow splitter. Samples are then directly loaded onto the microcapillary column by using a FAMOS inert capillary autosampler (LC Packings, San Francisco) after the flow split. Peptides are reconstituted in 6% acetic acid/0.01% TFA before injection.

### Analysis & Quantification.

Target protein.(e.g. a acetylated proteins of A-D above) in a biological sample is quantified using a validated AQUA peptide (as described above). The IAP method is then applied to the complex mixture of peptides derived from proteolytic cleavage of crude cell extracts to which the AQUA peptides have been spiked in.

LC-SRM of the entire sample is then carried out. MS/MS may be performed by using a ThermoFinnigan (San Jose, Calif.) mass spectrometer (LCQ DecaXP ion trap or TSQ Quantum triple quadrupole or LTQ). On the DecaXP, parent ions are isolated at 1.6 m/z width, the ion injection time being limited to 150 ms per microscan, with two microscans per peptide averaged, and with an AGC setting of 1×10⁸; on the Quantum, Q1 is kept at 0.4 and Q3 at 0.8 m/z with a scan time of 200 ms per peptide. On both instruments, analyte and internal standard are analyzed in alternation within a previously known reverse-phase retention window; well-resolved pairs of internal standard and analyte are analyzed in separate retention segments to improve duty cycle. Data are processed by integrating the appropriate peaks in an extracted ion chromatogram (60.15 m/z from the fragment monitored) for the native and internal standard, followed by calculation of the ratio of peak areas multiplied by the absolute amount of internal standard (e.g., 500 fmol).

What is claimed is:

1-47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

62. An isolated acetylation site-specific antibody that specifically binds a human acetylation signaling protein selected from Column A of Table 1, Rows 1, 126, 48, 143 and 200 only when acetylated at the lysine listed in corresponding Column D of Table 1, comprised within the acetylatable peptide sequence listed in corresponding Column E of Table 1 (SEQ ID NOs: 143, 145, 56, 162 and 225), wherein said antibody does not bind said signaling protein when not acetylated at said lysine.

63. An isolated acetylation site-specific antibody that specifically binds a human acetylation signaling protein selected from Column A of Table 1, Rows 1, 126, 48, 143 and 200 only when not acetylated at the lysine listed in corresponding Column D of Table 1, comprised within the acetylatable peptide sequence listed in corresponding Column E of Table 1 (SEQ ID NOs: 143, 145, 56, 162 and 225), wherein said antibody does not bind said signaling protein when acetylated at said lysine.

64. A method selected from the group consisting of:
(a) a method for detecting a human acetylation signaling protein selected from Column A of Table 1, Rows 1, 126, 48, 143 and 200 wherein said human acetylation signaling protein is acetylated at the lysine listed in corresponding Column D of Table 1, comprised within the acetylatable peptide sequence listed in corresponding Column E of Table 1 (SEQ ID NOs: 143, 145, 56, 162 and 225), comprising the step of adding an isolated acetylation-specific antibody according to claim 62, to a sample comprising said human acetylation signaling protein under conditions that permit the binding of said antibody to said human acetylation signaling protein, and detecting bound antibody;
(b) a method for quantifying the amount of a human acetylation signaling protein listed in Column A of Table 1, Rows 1, 126, 48, 143 and 200 that is acetylated at the corresponding lysine listed in Column D of Table 1, comprised within the acetylatable peptide sequence listed in corresponding Column E of Table 1 (SEQ ID NOs: 143, 145, 56, 162 and 225), in a sample using a heavy-isotope labeled peptide (AQUA™ peptide), said labeled peptide comprising a acetylated lysine at said corresponding lysine listed Column D of Table 1, comprised within the acetylatable peptide sequence listed in corresponding Column E of Table 1 as an internal standard; and
(c) a method comprising step (a) followed by step (b).

65. The method of claim 64, wherein said isolated acetylation-specific antibody is capable of specifically binding eEF1A-1 only when acetylated at K179, comprised within the acetylatable peptide sequence listed in Column E, Row 1, of Table 1 (SEQ ID NO: 143), wherein said antibody does not bind said protein when not acetylated at said lysine.

66. The method of claim 64, wherein said isolated acetylation-specific antibody is capable of specifically binding eEF1A-1 only when not acetylated at K179, comprised within the acetylatable peptide sequence listed in Column E, Row 1, of Table 1 (SEQ ID NO: 143), wherein said antibody does not bind said protein when acetylated at said lysine.

67. The method of claim 64, wherein said isolated acetylation-specific antibody is capable of specifically binding EEF1G only when acetylated at K 147, comprised within the acetylatable peptide sequence listed in Column E, Row 126, of Table 1 (SEQ ID NO: 145), wherein said antibody does not bind said protein when not acetylated at said lysine.

68. The method of claim 64, wherein said isolated acetylation-specific antibody is capable of specifically binding EEF1G only when not acetylated at K 147, comprised within the acetylatable peptide sequence listed in Column E, Row 126, of Table 1 (SEQ ID NO: 145), wherein said antibody does not bind said protein when acetylated at said lysine.

69. The method of claim 64, wherein said isolated acetylation-specific antibody is capable of specifically binding PDA6 only when acetylated at K102, comprised within the acetylatable peptide sequence listed in Column E, Row 48, of Table 1 (SEQ ID NO: 56), wherein said antibody does not bind said protein when not acetylated at said lysine.

70. The method of claim 64, wherein said isolated acetylation-specific antibody is capable of specifically binding PDA6 only when not acetylated at K102, comprised within the acetylatable peptide sequence listed in Column E, Row 48, of Table 1 (SEQ ID NO: 56), wherein said antibody does not bind said protein when acetylated at said lysine.

71. The method of claim 64, wherein said isolated acetylation-specific antibody is capable of specifically binding RPL24 only when acetylated at K27, comprised within the acetylatable peptide sequence listed in Column E, Row 143, of Table 1 (SEQ ID NO: 162), wherein said antibody does not bind said protein when not acetylated at said lysine.

72. The method of claim 64, wherein said isolated acetylation-specific antibody is capable of specifically binding RPL24 only when not acetylated at K27, comprised within the acetylatable peptide sequence listed in Column E, Row 143, of Table 1 (SEQ ID NO: 162), wherein said antibody does not bind said protein when acetylated at said lysine.

73. The method of claim 64, wherein said isolated acetylation-specific antibody is capable of specifically binding DOCK7 only when acetylated at K1931, comprised within the acetylatable peptide sequence listed in Column E, Row 200, of Table 1 (SEQ ID NO: 225), wherein said antibody does not bind said protein when not acetylated at said lysine.

74. The method of claim 64, wherein said isolated acetylation-specific antibody is capable of specifically binding DOCK7 only when not acetylated at K1931, comprised within the acetylatable peptide sequence listed in Column E, Row 200, of Table 1 (SEQ ID NO: 225), wherein said antibody does not bind said protein when acetylated at said lysine.


SEQUENCE LISTING (391 sequences)

SEQ ID NO: 1 - protein, 14 aa, Homo sapiens
  MOD_RES (10)..(10): Phosphorylated-Lys
VDSMLVQALKSGDW

SEQ ID NO: 2 - protein, 17 aa, Homo sapiens
  MOD_RES (17)..(17): Phosphorylated-Lys
PTTGVEDPAVKGAVQRK

SEQ ID NO: 3 - protein, 10 aa, Homo sapiens
  MOD_RES (3)..(3): Phosphorylated-Lys
KSKKHKKKSK

SEQ ID NO: 4 - protein, 10 aa, Homo sapiens
  MOD_RES (6)..(6): Phosphorylated-Lys
KSKKHKKKSK

SEQ ID NO: 5 - protein, 10 aa, Homo sapiens
  MOD_RES (7)..(7): Phosphorylated-Lys
KSKKHKKKSK

SEQ ID NO: 6 - protein, 19 aa, Homo sapiens
  MOD_RES (15)..(15): Phosphorylated-Lys
MLSEDLPSLELYVNKMKDK

SEQ ID NO: 7 - protein, 11 aa, Homo sapiens
  MOD_RES (9)..(9): Phosphorylated-Lys
ELLALCLKKIK

SEQ ID NO: 8 - protein, 27 aa, Homo sapiens
  MOD_RES (26)..(26): Phosphorylated-Lys
CFNPPPKPEPFQFGQSSQKPPVAGGKK

SEQ ID NO: 9 - protein, 17 aa, Homo sapiens
  MOD_RES (10)..(10): Phosphorylated-Lys
WFKEFPLNLKTVSERAK

SEQ ID NO: 10 - protein, 21 aa, Homo sapiens
  MOD_RES (9)..(9): Phosphorylated-Lys
QIYNPPSGKYSSNLGNFNYER

SEQ ID NO: 11 - protein, 11 aa, Homo sapiens
  MOD_RES (5)..(5): Phosphorylated-Lys
FQGGKAFGLLK

SEQ ID NO: 12 - protein, 19 aa, Homo sapiens
  MOD_RES (3)..(3): Phosphorylated-Lys
PIKGNTKISVANNQNSSQK

SEQ ID NO: 13 - protein, 30 aa, Homo sapiens
  MOD_RES (23)..(23): Phosphorylated-Lys
PAATMVTNVVRPVSSTPVPIASKPFPTSGR

SEQ ID NO: 14 - protein, 18 aa, Homo sapiens
  MOD_RES (6)..(6): Phosphorylated-Lys
SRKASKEQGPTPVEKKKK

SEQ ID NO: 15 - protein, 18 aa, Homo sapiens
  MOD_RES (18)..(18): Phosphorylated-Lys
SRKASKEQGPTPVEKKKK

SEQ ID NO: 16 - protein, 11 aa, Homo sapiens
  MOD_RES (2)..(2): Phosphorylated-Lys
KKKKLKDKKEK

SEQ ID NO: 17 - protein, 11 aa, Homo sapiens
  MOD_RES (4)..(4): Phosphorylated-Lys
KKKKLKDKKEK

SEQ ID NO: 18 - protein, 11 aa, Homo sapiens
  MOD_RES (8)..(8): Phosphorylated-Lys
KKKKLKDKKEK

SEQ ID NO: 19 - protein, 11 aa, Homo sapiens
  MOD_RES (9)..(9): Phosphorylated-Lys
KKKKLKDKKEK

SEQ ID NO: 20-26 - skipped (7 entries)

SEQ ID NO: 27 - protein, 22 aa, Homo sapiens
  MOD_RES (22)..(22): Phosphorylated-Lys
MTWTPRNRSEDEEEEENIDLEK

SEQ ID NO: 28 - protein, 11 aa, Homo sapiens
  MOD_RES (6)..(6): Phosphorylated-Lys
QNTVSKGPFSK

SEQ ID NO: 29 - protein, 13 aa, Homo sapiens
  MOD_RES (11)..(11): Phosphorylated-Lys
QNTVSKGPFSKVR

SEQ ID NO: 30-31 - skipped (2 entries)

SEQ ID NO: 32 - protein, 14 aa, Homo sapiens
  MOD_RES (8)..(8): Phosphorylated-Lys
ITDSVLLKAGSWAK

SEQ ID NO: 33 - protein, 14 aa, Homo sapiens
  MOD_RES (14)..(14): Phosphorylated-Lys
ITDSVLLKAGSWAK

SEQ ID NO: 34 - protein, 17 aa, Homo sapiens
  MOD_RES (14)..(14): Phosphorylated-Lys
DVDFEGTDEPIFGKKPR

SEQ ID NO: 35 - protein, 16 aa, Homo sapiens
  MOD_RES (6)..(6): Phosphorylated-Lys
LITSDKVENFHEEHEK

SEQ ID NO: 36 - protein, 13 aa, Homo sapiens
  MOD_RES (11)..(11): Phosphorylated-Lys
RKAEGDAKGGKAK

SEQ ID NO: 37 - protein, 26 aa, Homo sapiens
  MOD_RES (2)..(2): Phosphorylated-Lys
GKVEAAGPGGESEPTGSGGTLAHTPR

SEQ ID NO: 38 - protein, 10 aa, Homo sapiens
  MOD_RES (5)..(5): Phosphorylated-Lys
TITGKTFSSR

SEQ ID NO: 39 - protein, 11 aa, Homo sapiens
  MOD_RES (10)..(10): Phosphorylated-Lys
INSITVDNCKK

SEQ ID NO: 40 - skipped

SEQ ID NO: 41 - protein, 26 aa, Homo sapiens
  MOD_RES (22)..(22): Phosphorylated-Lys
AQPVQVAEGSEPDGFWEALGGKAAYR

SEQ ID NO: 42 - protein, 11 aa, Homo sapiens
  MOD_RES (4)..(4): Phosphorylated-Lys
LLQKVFNDTCR

SEQ ID NO: 43 - protein, 19 aa, Homo sapiens
  MOD_RES (11)..(11): Phosphorylated-Lys
KELEFEQKLYKEKEEMLEK

SEQ ID NO: 44 - protein, 14 aa, Homo sapiens
  MOD_RES (8)..(8): Phosphorylated-Lys
KLQNQNYKGHGLSK

SEQ ID NO: 45 - protein, 14 aa, Homo sapiens
  MOD_RES (13)..(13): Phosphorylated-Lys
IGVATAADTLVTKF

SEQ ID NO: 46 - protein, 16 aa, Homo sapiens
  MOD_RES (4)..(4): Phosphorylated-Lys
LITKDLSEPYSIYTYR

SEQ ID NO: 47 - protein, 9 aa, Homo sapiens
  MOD_RES (3)..(3): Phosphorylated-Lys
NGKQWLLGK

SEQ ID NO: 48 - protein, 15 aa, Homo sapiens
  MOD_RES (6)..(6): Phosphorylated-Lys
MSSVQKMMQAGKKFK

SEQ ID NO: 49 - protein, 15 aa, Homo sapiens
  MOD_RES (12)..(12): Phosphorylated-Lys
MSSVQKMMQAGKKFK

SEQ ID NO: 50 - protein, 16 aa, Homo sapiens
  MOD_RES (2)..(2): Phosphorylated-Lys
QKRLEERAAMDAVCAK

SEQ ID NO: 51 - protein, 11 aa, Homo sapiens
  MOD_RES (3)..(3): Phosphorylated-Lys
ETKSFFTSQQL

SEQ ID NO: 52 - protein, 10 aa, Homo sapiens
  MOD_RES (8)..(8): Phosphorylated-Lys
FGALTAEKLR

SEQ ID NO: 53 - protein, 17 aa, Homo sapiens
  MOD_RES (8)..(8): Phosphorylated-Lys
NAQLDCSKLETLGIGQR

SEQ ID NO: 54 - protein, 19 aa, Homo sapiens
  MOD_RES (4)..(4): Phosphorylated-Lys
AVHKEFQQNNWHAVGCGFR

SEQ ID NO: 55 - protein, 21 aa, Homo sapiens
  MOD_RES (6)..(6): Phosphorylated-Lys
GFPTIKIFQKGESPVDYDGGR

SEQ ID NO: 56 - protein, 31 aa, Homo sapiens
  MOD_RES (25)..(25): Phosphorylated-Lys
VGAVDADKHHSLGGQYGVQGFPTIKIFGSNK

SEQ ID NO: 57 - protein, 13 aa, Homo sapiens
  MOD_RES (5)..(5): Phosphorylated-Lys
QGVGKYINPAATK

SEQ ID NO: 58 - protein, 15 aa, Homo sapiens
  MOD_RES (5)..(5): Phosphorylated-Lys
GGCYKHTFYGIESHR

SEQ ID NO: 59 - protein, 10 aa, Homo sapiens
  MOD_RES (3)..(3): Phosphorylated-Lys
QQKAVKIKTK

SEQ ID NO: 60 - protein, 8 aa, Homo sapiens
  MOD_RES (2)..(2): Phosphorylated-Lys
RKEKLKGK

SEQ ID NO: 61 - protein, 8 aa, Homo sapiens
  MOD_RES (4)..(4): Phosphorylated-Lys
RKEKLKGK

SEQ ID NO: 62 - protein, 10 aa, Homo sapiens
  MOD_RES (2)..(2): Phosphorylated-Lys
VKAKDVDEYK

SEQ ID NO: 63 - protein, 10 aa, Homo sapiens
  MOD_RES (4)..(4): Phosphorylated-Lys
VKAKDVDEYK

SEQ ID NO: 64 - protein, 13 aa, Homo sapiens
  MOD_RES (6)..(6): Phosphorylated-Lys
LKPNTKIMMMGTR

SEQ ID NO: 65 - protein, 12 aa, Homo sapiens
  MOD_RES (6)..(6): Phosphorylated-Lys
FSGFGKIFKPWK

SEQ ID NO: 66 - protein, 9 aa, Homo sapiens
  MOD_RES (9)..(9): Phosphorylated-Lys
AFTPFSGPK

SEQ ID NO: 67 - protein, 13 aa, Homo sapiens
  MOD_RES (3)..(3): Phosphorylated-Lys
ESKFFEHFIEGGR

SEQ ID NO: 68 - protein, 19 aa, Homo sapiens
  MOD_RES (14)..(14): Phosphorylated-Lys
HTLDGAACLLNSNKYFPSR

SEQ ID NO: 69 - protein, 14 aa, Homo sapiens
  MOD_RES (7)..(7): Phosphorylated-Lys
MPSDLAKKKAAKKK

SEQ ID NO: 70 - protein, 14 aa, Homo sapiens
  MOD_RES (8)..(8): Phosphorylated-Lys
MPSDLAKKKAAKKK

SEQ ID NO: 71 - protein, 14 aa, Homo sapiens
  MOD_RES (9)..(9): Phosphorylated-Lys
MPSDLAKKKAAKKK

SEQ ID NO: 72 - protein, 22 aa, Homo sapiens
  MOD_RES (4)..(4): Phosphorylated-Lys
GADKVNTFSALLLEPYKPPSAQ

SEQ ID NO: 73 - protein, 20 aa, Homo sapiens
  MOD_RES (4)..(4): Phosphorylated-Lys
MGDKKDDKDSPKKNKGKERR

SEQ ID NO: 74 - protein, 20 aa, Homo sapiens
  MOD_RES (5)..(5): Phosphorylated-Lys
MGDKKDDKDSPKKNKGKERR

SEQ ID NO: 75 - protein, 17 aa, Homo sapiens
  MOD_RES (9)..(9): Phosphorylated-Lys
QLVLNVWGKVEADIPGH

SEQ ID NO: 76 - protein, 16 aa, Homo sapiens
  MOD_RES (13)..(13): Phosphorylated-Lys
NVGFESDTGGAFKGFK

SEQ ID NO: 77 - protein, 16 aa, Homo sapiens
  MOD_RES (12)..(12): Phosphorylated-Lys
GPAGDATVASEKESVM

SEQ ID NO: 78-79 - skipped (2 entries)

SEQ ID NO: 80 - protein, 14 aa, Homo sapiens
  MOD_RES (4)..(4): Phosphorylated-Lys
DKYKQIFLGGVDKR

SEQ ID NO: 81 - skipped

SEQ ID NO: 82 - protein, 8 aa, Homo sapiens
  MOD_RES (4)..(4): Phosphorylated-Lys
GVCKCFCR

SEQ ID NO: 83 - protein, 12 aa, Homo sapiens
  MOD_RES (5)..(5): Phosphorylated-Lys
KPEAKFFTTPSK

SEQ ID NO: 84 - protein, 12 aa, Homo sapiens
  MOD_RES (7)..(7): Phosphorylated-Lys
NKVAGGKMKKRK

SEQ ID NO: 85 - protein, 12 aa, Homo sapiens
  MOD_RES (10)..(10): Phosphorylated-Lys
NKVAGGKMKKRK

SEQ ID NO: 86 - protein, 12 aa, Homo sapiens
  MOD_RES (4)..(4): Phosphorylated-Lys
KFIKNKAWSKQK

SEQ ID NO: 87 - protein, 12 aa, Homo sapiens
  MOD_RES (6)..(6): Phosphorylated-Lys
KFIKNKAWSKQK

SEQ ID NO: 88 - protein, 28 aa, Homo sapiens
  MOD_RES (27)..(27): Phosphorylated-Lys
NTNTINNGTQQQAQSMTTTIKPGDDWKK

SEQ ID NO: 89 - protein, 9 aa, Homo sapiens
  MOD_RES (5)..(5): Phosphorylated-Lys
LLLFKTFSR

SEQ ID NO: 90 - protein, 14 aa, Homo sapiens
  MOD_RES (13)..(13): Phosphorylated-Lys
KVVVVVPNEEDWKK

SEQ ID NO: 91 - protein, 13 aa, Homo sapiens
  MOD_RES (6)..(6): Phosphorylated-Lys
YLEAIKIFSGPLK

SEQ ID NO: 92 - protein, 8 aa, Homo sapiens
  MOD_RES (8)..(8): Phosphorylated-Lys
KVKGLGPK

SEQ ID NO: 93 - protein, 14 aa, Homo sapiens
  MOD_RES (6)..(6): Phosphorylated-Lys
SGFNSKSGKRGSSK

SEQ ID NO: 94 - protein, 14 aa, Homo sapiens
  MOD_RES (9)..(9): Phosphorylated-Lys
SGFNSKSGKRGSSK

SEQ ID NO: 95 - protein, 14 aa, Homo sapiens
  MOD_RES (14)..(14): Phosphorylated-Lys
SGFNSKSGKRGSSK

SEQ ID NO: 96 - protein, 23 aa, Homo sapiens
  MOD_RES (22)..(22): Phosphorylated-Lys
IGGDAATTVNNSTPDFGFGGQKR

SEQ ID NO: 97 - protein, 13 aa, Homo sapiens
  MOD_RES (12)..(12): Phosphorylated-Lys
KLLEDTLFPSSKK

SEQ ID NO: 98 - protein, 11 aa, Homo sapiens
  MOD_RES (10)..(10): Phosphorylated-Lys
KVGMRHYHLKR

SEQ ID NO: 99 - protein, 15 aa, Homo sapiens
  MOD_RES (1)..(1): Phosphorylated-Lys
KPKAKKADVGGKVKK

SEQ ID NO: 100 - protein, 15 aa, Homo sapiens
  MOD_RES (3)..(3): Phosphorylated-Lys
KPKAKKADVGGKVKK

SEQ ID NO: 101 - protein, 15 aa, Homo sapiens
  MOD_RES (6)..(6): Phosphorylated-Lys
KPKAKKADVGGKVKK

SEQ ID NO: 102 - skipped

SEQ ID NO: 103 - protein, 19 aa, Homo sapiens
  MOD_RES (9)..(9): Phosphorylated-Lys
FSPTDFLAKMEEANIQPNR

SEQ ID NO: 104 - protein, 16 aa, Homo sapiens
  MOD_RES (16)..(16): Phosphorylated-Lys
SENQEFYQDTFGQQWK

SEQ ID NO: 105 - protein, 10 aa, Homo sapiens
  MOD_RES (9)..(9): Phosphorylated-Lys
IPTPIVGVKR

SEQ ID NO: 106 - protein, 15 aa, Homo sapiens
  MOD_RES (9)..(9): Phosphorylated-Lys
PLSQEPAKKVKAKKK

SEQ ID NO: 107 - protein, 15 aa, Homo sapiens
  MOD_RES (11)..(11): Phosphorylated-Lys
PLSQEPAKKVKAKKK

SEQ ID NO: 108 - protein, 15 aa, Homo sapiens
  MOD_RES (14)..(14): Phosphorylated-Lys
PLSQEPAKKVKAKKK

SEQ ID NO: 109 - protein, 15 aa, Homo sapiens
  MOD_RES (8)..(8): Phosphorylated-Lys
PLSQEPAKKVKAKKK

SEQ ID NO: 110 - protein, 13 aa, Homo sapiens
  MOD_RES (7)..(7): Phosphorylated-Lys
SPYSGPKFNSAIR

SEQ ID NO: 111-113 - skipped (3 entries)

SEQ ID NO: 114 - protein, 11 aa, Homo sapiens
  MOD_RES (9)..(9): Phosphorylated-Lys
VIEGSLSPKER

SEQ ID NO: 115 - protein, 12 aa, Homo sapiens
  MOD_RES (11)..(11): Phosphorylated-Lys
RKGKAQYVLAKR

SEQ ID NO: 116 - protein, 13 aa, Homo sapiens
  MOD_RES (8)..(8): Phosphorylated-Lys
DKLKDEPKIKLLK

SEQ ID NO: 117 - protein, 13 aa, Homo sapiens
  MOD_RES (5)..(5): Phosphorylated-Lys
DLMDKTQKVKVKK

SEQ ID NO: 118 - protein, 13 aa, Homo sapiens
  MOD_RES (8)..(8): Phosphorylated-Lys
DLMDKTQKVKVKK

SEQ ID NO: 119 - protein, 13 aa, Homo sapiens
  MOD_RES (10)..(10): Phosphorylated-Lys
DLMDKTQKVKVKK

SEQ ID NO: 120 - protein, 12 aa, Homo sapiens
  MOD_RES (11)..(11): Phosphorylated-Lys
KGPELPLVPVKR

SEQ ID NO: 121 - protein, 16 aa, Homo sapiens
  MOD_RES (15)..(15): Phosphorylated-Lys
QLVSKPLSSSVSNNKR

SEQ ID NO: 122 - protein, 17 aa, Homo sapiens
  MOD_RES (4)..(4): Phosphorylated-Lys
DIGKKLMSCPLAGLISK

SEQ ID NO: 123 - protein, 11 aa, Homo sapiens
  MOD_RES (7)..(7): Phosphorylated-Lys
ELNGLKKLKRK

SEQ ID NO: 124 - protein, 11 aa, Homo sapiens
  MOD_RES (11)..(11): Phosphorylated-Lys
ELNGLKKLKRK

SEQ ID NO: 125 - protein, 22 aa, Homo sapiens
  MOD_RES (16)..(16): Phosphorylated-Lys
KLTALDYHNPAGFNCKDETEFR

SEQ ID NO: 126 - protein, 20 aa, Homo sapiens
  MOD_RES (4)..(4): Phosphorylated-Lys
CYRKYKQLTWIALKFALYKK

SEQ ID NO: 127 - protein, 20 aa, Homo sapiens
  MOD_RES (6)..(6): Phosphorylated-Lys
CYRKYKQLTWIALKFALYKK

SEQ ID NO: 128 - protein, 20 aa, Homo sapiens
  MOD_RES (14)..(14): Phosphorylated-Lys
CYRKYKQLTWIALKFALYKK

SEQ ID NO: 129 - protein, 20 aa, Homo sapiens
  MOD_RES (19)..(19): Phosphorylated-Lys
CYRKYKQLTWIALKFALYKK

SEQ ID NO: 130 - protein, 11 aa, Homo sapiens
  MOD_RES (10)..(10): Phosphorylated-Lys
ATWETILDGKR

SEQ ID NO: 131 - skipped

SEQ ID NO: 132 - protein, 12 aa, Homo sapiens
  MOD_RES (11)..(11): Phosphorylated-Lys
AKFKFSLLNAKR

SEQ ID NO: 133 - protein, 14 aa, Homo sapiens
  MOD_RES (14)..(14): Phosphorylated-Lys
SGMACKTTATISSK

SEQ ID NO: 134 - protein, 13 aa, Homo sapiens
  MOD_RES (10)..(10): Phosphorylated-Lys
LVKDQEALMKSVK

SEQ ID NO: 135 - protein, 13 aa, Homo sapiens
  MOD_RES (6)..(6): Phosphorylated-Lys
SVVQVKNFFVNYR

SEQ ID NO: 136 - protein, 14 aa, Homo sapiens
  MOD_RES (1)..(1): Phosphorylated-Lys
KNQLVQKAILQKNK

SEQ ID NO: 137 - protein, 14 aa, Homo sapiens
  MOD_RES (7)..(7): Phosphorylated-Lys
KNQLVQKAILQKNK

SEQ ID NO: 138 - protein, 11 aa, Homo sapiens
  MOD_RES (8)..(8): Phosphorylated-Lys
LACDTCGKSFK

SEQ ID NO: 139 - protein, 13 aa, Homo sapiens
  MOD_RES (12)..(12): Phosphorylated-Lys
EQQSNIAVFQGKK

SEQ ID NO: 140 - protein, 18 aa, Homo sapiens
  MOD_RES (8)..(8): Phosphorylated-Lys
SHVATCSKYQNYIMEGVK

SEQ ID NO: 141 - protein, 12 aa, Homo sapiens
  MOD_RES (3)..(3): Phosphorylated-Lys
SGKAFNCSSLLR

SEQ ID NO: 142 - skipped

SEQ ID NO: 143 - protein, 14 aa, Homo sapiens
  MOD_RES (13)..(13): Phosphorylated-Lys
YEEIVKEVSTYIKK

SEQ ID NO: 144 - skipped

SEQ ID NO: 145 - protein, 12 aa, Homo sapiens
  MOD_RES (10)..(10): Phosphorylated-Lys
ILGLLDAYLKTR

SEQ ID NO: 146 - protein, 11 aa, Homo sapiens
  MOD_RES (4)..(4): Phosphorylated-Lys
LCEKMAQFDAK

SEQ ID NO: 147 - protein, 13 aa, Homo sapiens
  MOD_RES (11)..(11): Phosphorylated-Lys
VFSGLVSTGLKVR

SEQ ID NO: 148 - protein, 13 aa, Homo sapiens
  MOD_RES (9)..(9): Phosphorylated-Lys
QFAEMYVAKFAAK

SEQ ID NO: 149 - protein, 9 aa, Homo sapiens
  MOD_RES (4)..(4): Phosphorylated-Lys
KYSKIFEQK

SEQ ID NO: 150 - protein, 10 aa, Homo sapiens
  MOD_RES (4)..(4): Phosphorylated-Lys
QIHKFEELNR

SEQ ID NO: 151 - protein, 15 aa, Homo sapiens
  MOD_RES (9)..(9): Phosphorylated-Lys
KPEENPASKFSSASK

SEQ ID NO: 152 - protein, 13 aa, Homo sapiens
  MOD_RES (12)..(12): Phosphorylated-Lys
VHLVGIDIFTGKK

SEQ ID NO: 153 - protein, 29 aa, Homo sapiens
  MOD_RES (28)..(28): Phosphorylated-Lys
GPTQPPTLPAGSGSNDETCTGAGYPQGKR

SEQ ID NO: 154 - protein, 7 aa, Homo sapiens
  MOD_RES (5)..(5): Phosphorylated-Lys
GPLDKWR

SEQ ID NO: 155 - protein, 22 aa, Homo sapiens
  MOD_RES (6)..(6): Phosphorylated-Lys
STPRPKFSVCVLGDQQHCDEAK

SEQ ID NO: 156 - protein, 7 aa, Homo sapiens
  MOD_RES (5)..(5): Phosphorylated-Lys
GPLDKWR

SEQ ID NO: 157 - protein, 16 aa, Homo sapiens
  MOD_RES (14)..(14): Phosphorylated-Lys
VLEQLTGQTPVFSKAR

SEQ ID NO: 158 - protein, 10 aa, Homo sapiens
  MOD_RES (9)..(9): Phosphorylated-Lys
IGPLGLSPKK

SEQ ID NO: 159 - protein, 13 aa, Homo sapiens
  MOD_RES (7)..(7): Phosphorylated-Lys
LAAIVAKQVLLGR

SEQ ID NO: 160 - protein, 13 aa, Homo sapiens
  MOD_RES (5)..(5): Phosphorylated-Lys
CAQAKQWGWTQGR

SEQ ID NO: 161 - protein, 9 aa, Homo sapiens
  MOD_RES (2)..(2): Phosphorylated-Lys
VKNFGIWLR

SEQ ID NO: 162 - protein, 12 aa, Homo sapiens
  MOD_RES (4)..(4): Phosphorylated-Lys
TDGKVFQFLNAK

SEQ ID NO: 163 - protein, 11 aa, Homo sapiens
  MOD_RES (7)..(7): Phosphorylated-Lys
KPAPEKKPAEK

SEQ ID NO: 164 - protein, 12 aa, Homo sapiens
  MOD_RES (11)..(11): Phosphorylated-Lys
YLMEEDEDAYKK

SEQ ID NO: 165 - protein, 9 aa, Homo sapiens
  MOD_RES (3)..(3): Phosphorylated-Lys
EQKKGKGLR

SEQ ID NO: 166 - protein, 22 aa, Homo sapiens
  MOD_RES (19)..(19): Phosphorylated-Lys
YVASYLLAALGGNSSPSAKDIK

SEQ ID NO: 167 - protein, 9 aa, Homo sapiens
  MOD_RES (5)..(5): Phosphorylated-Lys
VIIEKYYTR

SEQ ID NO: 168 - protein, 9 aa, Homo sapiens
  MOD_RES (7)..(7): Phosphorylated-Lys
ADGIVSKNF

SEQ ID NO: 169 - protein, 31 aa, Homo sapiens
  MOD_RES (3)..(3): Phosphorylated-Lys
IVKHAFEIIHLLTGENPLQVLVNAIINSGPR

SEQ ID NO: 170 - protein, 30 aa, Homo sapiens
  MOD_RES (30)..(30): Phosphorylated-Lys
IVKPNGEKPDEFESGISQALLELEMNSDLK

SEQ ID NO: 171 - protein, 16 aa, Homo sapiens
  MOD_RES (12)..(12): Phosphorylated-Lys
AIIIFVPVPQLKSFQK

SEQ ID NO: 172 - protein, 18 aa, Homo sapiens
  MOD_RES (9)..(9): Phosphorylated-Lys
TTLTAAITKILAEGGGAK

SEQ ID NO: 173 - protein, 11 aa, Homo sapiens
  MOD_RES (5)..(5): Phosphorylated-Lys
QRKKKKTKNKK

SEQ ID NO: 174 - protein, 20 aa, Homo sapiens
  MOD_RES (16)..(16): Phosphorylated-Lys
SLLHSPGDYILLSADKYEIK

SEQ ID NO: 175 - protein, 15 aa, Homo sapiens
  MOD_RES (15)..(15): Phosphorylated-Lys
FRATAFQREELMKMK

SEQ ID NO: 176 - protein, 12 aa, Homo sapiens
  MOD_RES (8)..(8): Phosphorylated-Lys
STGYDPVKLFTK

SEQ ID NO: 177 - protein, 9 aa, Homo sapiens
  MOD_RES (9)..(9): Phosphorylated-Lys
KENQWCEEK

SEQ ID NO: 178 - protein, 17 aa, Homo sapiens
  MOD_RES (7)..(7): Phosphorylated-Lys
ICLDILKDKWSPALQIR

SEQ ID NO: 179 - protein, 13 aa, Homo sapiens
  MOD_RES (5)..(5): Phosphorylated-Lys
LLLGKDFPASPPK

SEQ ID NO: 180 - protein, 20 aa, Homo sapiens
  MOD_RES (4)..(4): Phosphorylated-Lys
TSAKEEDAFHFVSYVPVNGR

SEQ ID NO: 181 - skipped

SEQ ID NO: 182 - protein, 11 aa, Homo sapiens
  MOD_RES (9)..(9): Phosphorylated-Lys
DTDMNKKDKQK

SEQ ID NO: 183 - protein, 13 aa, Homo sapiens
  MOD_RES (6)..(6): Phosphorylated-Lys
NSSYGKVEGFWSK

SEQ ID NO: 184 - protein, 9 aa, Homo sapiens
  MOD_RES (1)..(1): Phosphorylated-Lys
KANLNVLDR

SEQ ID NO: 185 - protein, 18 aa, Homo sapiens
  MOD_RES (12)..(12): Phosphorylated-Lys
STVTLSWKPVQKVEIGQK

SEQ ID NO: 186 - protein, 15 aa, Homo sapiens
  MOD_RES (14)..(14): Phosphorylated-Lys
TANQASDTFSGIGKK

SEQ ID NO: 187 - protein, 14 aa, Homo sapiens
  MOD_RES (14)..(14): Phosphorylated-Lys
SFVGGLELIVNLLK

SEQ ID NO: 188 - protein, 13 aa, Homo sapiens
  MOD_RES (11)..(11): Phosphorylated-Lys
YATLSLFNTYKGK

SEQ ID NO: 189 - skipped

SEQ ID NO: 190 - protein, 16 aa, Homo sapiens
  MOD_RES (9)..(9): Phosphorylated-Lys
GHCPPAPAKPMHPENK

SEQ ID NO: 191 - protein, 17 aa, Homo sapiens
  MOD_RES (16)..(16): Phosphorylated-Lys
ENSQLDTSPPCLGGNKK

SEQ ID NO: 192 - protein, 12 aa, Homo sapiens
  MOD_RES (10)..(10): Phosphorylated-Lys
ENDEEMKAAKEK

SEQ ID NO: 193 - skipped

SEQ ID NO: 194 - protein, 12 aa, Homo sapiens
  MOD_RES (9)..(9): Phosphorylated-Lys
PKPGGAGGKGMR

SEQ ID NO: 195 - protein, 26 aa, Homo sapiens
  MOD_RES (16)..(16): Phosphorylated-Lys
NAMLIKGGNANATVTKVLKDVYALKK

SEQ ID NO: 196 - protein, 26 aa, Homo sapiens
  MOD_RES (25)..(25): Phosphorylated-Lys
NAMLIKGGNANATVTKVLKDVYALKK

SEQ ID NO: 197 - protein, 18 aa, Homo sapiens
  MOD_RES (14)..(14): Phosphorylated-Lys
LQHINPLLPACLNKEESK

SEQ ID NO: 198 - protein, 12 aa, Homo sapiens
  MOD_RES (11)..(11): Phosphorylated-Lys
DAIAQAEMDLKR

SEQ ID NO: 199 - protein, 20 aa, Homo sapiens
  MOD_RES (19)..(19): Phosphorylated-Lys
SCSQLLLYELQSAVSEENKK

SEQ ID NO: 200-201 - skipped (2 entries)

SEQ ID NO: 202 - protein, 12 aa, Homo sapiens
  MOD_RES (8)..(8): Phosphorylated-Lys
ANYESPGKIFLR

SEQ ID NO: 203 - protein, 11 aa, Homo sapiens
  MOD_RES (4)..(4): Phosphorylated-Lys
SGNKFFSMPLK

SEQ ID NO: 204 - protein, 13 aa, Homo sapiens
  MOD_RES (12)..(12): Phosphorylated-Lys
GGPGSTLSFVGKR

SEQ ID NO: 205 - protein, 16 aa, Homo sapiens
  MOD_RES (6)..(6): Phosphorylated-Lys
RKRMEKMAEQQAVRER

SEQ ID NO: 206 - protein, 11 aa, Homo sapiens
  MOD_RES (6)..(6): Phosphorylated-Lys
YLTNQKNSNSK

SEQ ID NO: 207 - protein, 14 aa, Homo sapiens
  MOD_RES (11)..(11): Phosphorylated-Lys
YLTNQKNSNSKNDR

SEQ ID NO: 208 - protein, 24 aa, Homo sapiens
  MOD_RES (3)..(3): Phosphorylated-Lys
VLKEFHQAGKPIGLCCIAPVLAAK

SEQ ID NO: 209 - protein, 11 aa, Homo sapiens
  MOD_RES (6)..(6): Phosphorylated-Lys
EQWLQKIEAIK

SEQ ID NO: 210 - protein, 9 aa, Homo sapiens
  MOD_RES (3)..(3): Phosphorylated-Lys
REKLLGWLR

SEQ ID NO: 211 - protein, 12 aa, Homo sapiens
  MOD_RES (9)..(9): Phosphorylated-Lys
NVFHIASQKNFK

SEQ ID NO: 212 - protein, 10 aa, Homo sapiens
  MOD_RES (1)..(1): Phosphorylated-Lys
KLNLDLAGLR

SEQ ID NO: 213 - protein, 25 aa, Homo sapiens
  MOD_RES (17)..(17): Phosphorylated-Lys
ASAQQENSSTCIGSAIKSESGNSAR

SEQ ID NO: 214 - skipped

SEQ ID NO: 215 - protein, 8 aa, Homo sapiens
  MOD_RES (1)..(1): Phosphorylated-Lys
KDFISVLR

SEQ ID NO: 216 - protein, 11 aa, Homo sapiens
  MOD_RES (8)..(8): Phosphorylated-Lys
EENLFSWKGAK

SEQ ID NO: 217 - protein, 8 aa, Homo sapiens
  MOD_RES (5)..(5): Phosphorylated-Lys
CVGQKYEK

SEQ ID NO: 218 - protein, 24 aa, Homo sapiens
  MOD_RES (1)..(1): Phosphorylated-Lys
KVMSSIVNDLANSIWEGSSQGQTK

SEQ ID NO: 219 - protein, 24 aa, Homo sapiens
  MOD_RES (24)..(24): Phosphorylated-Lys
KVMSSIVNDLANSIWEGSSQGQTK

SEQ ID NO: 220 - protein, 16 aa, Homo sapiens
  MOD_RES (15)..(15): Phosphorylated-Lys
CVGNFGNSSGEVDTKK

SEQ ID NO: 221 - protein, 10 aa, Homo sapiens
  MOD_RES (4)..(4): Phosphorylated-Lys
SILKVVINNK

SEQ ID NO: 222 - protein, 10 aa, Homo sapiens
  MOD_RES (10)..(10): Phosphorylated-Lys
SILKVVINNK

SEQ ID NO: 223 - protein, 10 aa, Homo sapiens
  MOD_RES (5)..(5): Phosphorylated-Lys
LTSGKLENLR

SEQ ID NO: 224 - protein, 12 aa, Homo sapiens
  MOD_RES (10)..(10): Phosphorylated-Lys
KFIKNKAWSKQK

SEQ ID NO: 225 - protein, 11 aa, Homo sapiens
  MOD_RES (10)..(10): Phosphorylated-Lys
AHGELHEQFKR

SEQ ID NO: 226 - protein, 11 aa, Homo sapiens
  MOD_RES (5)..(5): Phosphorylated-Lys
EMQDKVLDMEK

SEQ ID NO: 227 - protein, 14 aa, Homo sapiens
  MOD_RES (2)..(2): Phosphorylated-Lys
TKTVLSLFDEEEDK

SEQ ID NO: 228 - protein, 12 aa, Homo sapiens
  MOD_RES (5)..(5): Phosphorylated-Lys
GILGKFVPPIPK

SEQ ID NO: 229 - protein, 9 aa, Homo sapiens
  MOD_RES (8)..(8): Phosphorylated-Lys
TEQTEDLKR

SEQ ID NO: 230 - protein, 13 aa, Homo sapiens
  MOD_RES (8)..(8): Phosphorylated-Lys
LHIRSINKGGQLR

SEQ ID NO: 231 - protein, 10 aa, Homo sapiens
  MOD_RES (10)..(10): Phosphorylated-Lys
KNMNWKQLPK

SEQ ID NO: 232 - protein, 24 aa, Homo sapiens
  MOD_RES (22)..(22): Phosphorylated-Lys
GQHEPSKPPPAGETVTGGFGAKKR

SEQ ID NO: 233 - protein, 9 aa, Homo sapiens
  MOD_RES (9)..(9): Phosphorylated-Lys
GTLIDNQFK

SEQ ID NO: 234 - skipped

SEQ ID NO: 235 - protein, 19 aa, Homo sapiens
  MOD_RES (3)..(3): Phosphorylated-Lys
ITKKNPKKAEKLERKKVYR

SEQ ID NO: 236 - protein, 19 aa, Homo sapiens
  MOD_RES (7)..(7): Phosphorylated-Lys
ITKKNPKKAEKLERKKVYR

SEQ ID NO: 237 - protein, 19 aa, Homo sapiens
  MOD_RES (8)..(8): Phosphorylated-Lys
ITKKNPKKAEKLERKKVYR

SEQ ID NO: 238 - protein, 19 aa, Homo sapiens
  MOD_RES (11)..(11): Phosphorylated-Lys
ITKKNPKKAEKLERKKVYR

SEQ ID NO: 239 - protein, 19 aa, Homo sapiens
  MOD_RES (15)..(15): Phosphorylated-Lys
ITKKNPKKAEKLERKKVYR

SEQ ID NO: 240 - protein, 17 aa, Homo sapiens
  MOD_RES (6)..(6): Phosphorylated-Lys
LLLQTKEDYEALIKSLK

SEQ ID NO: 241 - protein, 17 aa, Homo sapiens
  MOD_RES (14)..(14): Phosphorylated-Lys
LLLQTKEDYEALIKSLK

SEQ ID NO: 242 - protein, 9 aa, Homo sapiens
  MOD_RES (4)..(4): Phosphorylated-Lys
AEEKLESYR

SEQ ID NO: 243 - protein, 11 aa, Homo sapiens
  MOD_RES (3)..(3): Phosphorylated-Lys
RKKGGTYKDRK

SEQ ID NO: 244 - protein, 11 aa, Homo sapiens
  MOD_RES (11)..(11): Phosphorylated-Lys
RKKGGTYKDRK

SEQ ID NO: 245 - protein, 14 aa, Homo sapiens
  MOD_RES (3)..(3): Phosphorylated-Lys
FTKKSNSGFLRGKR

SEQ ID NO: 246 - protein, 14 aa, Homo sapiens
  MOD_RES (4)..(4): Phosphorylated-Lys
FTKKSNSGFLRGKR

SEQ ID NO: 247 - protein, 16 aa, Homo sapiens
  MOD_RES (13)..(13): Phosphorylated-Lys
LVEEVTDDKTLPKLER

SEQ ID NO: 248 - skipped

SEQ ID NO: 249 - protein, 17 aa, Homo sapiens
  MOD_RES (6)..(6): Phosphorylated-Lys
ATVVGKPEKTFFLEALR

SEQ ID NO: 250 - protein, 12 aa, Homo sapiens
  MOD_RES (7)..(7): Phosphorylated-Lys
LLTWDVKDTLLR

SEQ ID NO: 251 - protein, 23 aa, Homo sapiens
  MOD_RES (4)..(4): Phosphorylated-Lys
LLCKNFGAENPDPFVPVLNTAVK

SEQ ID NO: 252 - protein, 15 aa, Homo sapiens
  MOD_RES (8)..(8): Phosphorylated-Lys
SINNYNPKDFDWNLK

SEQ ID NO: 253 - protein, 18 aa, Homo sapiens
  MOD_RES (15)..(15): Phosphorylated-Lys
GSASTPVQGSIPEGKPLR

SEQ ID NO: 254 - protein, 25 aa, Homo sapiens
  MOD_RES (1)..(1): Phosphorylated-Lys
KGPGPGGGVSGGKAEAEAASEVWCR

SEQ ID NO: 255 - protein, 10 aa, Homo sapiens
  MOD_RES (6)..(6): Phosphorylated-Lys
GWALLKFGGK

SEQ ID NO: 256 - protein, 10 aa, Homo sapiens
  MOD_RES (4)..(4): Phosphorylated-Lys
KKDKKGKKGK

SEQ ID NO: 257 - protein, 10 aa, Homo sapiens
  MOD_RES (5)..(5): Phosphorylated-Lys
KKDKKGKKGK

SEQ ID NO: 258 - protein, 10 aa, Homo sapiens
  MOD_RES (7)..(7): Phosphorylated-Lys
KKDKKGKKGK

SEQ ID NO: 259 - protein, 23 aa, Homo sapiens
  MOD_RES (3)..(3): Phosphorylated-Lys
LIKEPAPDSGLLGLFQGQNSLLH

SEQ ID NO: 260 - protein, 18 aa, Homo sapiens
  MOD_RES (16)..(16): Phosphorylated-Lys
VLPGSSVLFLCDMQEKFR

SEQ ID NO: 261 - protein, 11 aa, Homo sapiens
  MOD_RES (3)..(3): Phosphorylated-Lys
GFKAFPGKGER

SEQ ID NO: 262 - protein, 11 aa, Homo sapiens
  MOD_RES (8)..(8): Phosphorylated-Lys
GFKAFPGKGER

SEQ ID NO: 263 - protein, 21 aa, Homo sapiens
  MOD_RES (6)..(6): Phosphorylated-Lys
APIPQKQEILVEPEPLFGAPK

SEQ ID NO: 264-265 - skipped (2 entries)

SEQ ID NO: 266 - protein, 12 aa, Homo sapiens
  MOD_RES (2)..(2): Phosphorylated-Lys
EKKKNLAGGKKR

SEQ ID NO: 267 - protein, 12 aa, Homo sapiens
  MOD_RES (4)..(4): Phosphorylated-Lys
EKKKNLAGGKKR

SEQ ID NO: 268 - protein, 12 aa, Homo sapiens
  MOD_RES (11)..(11): Phosphorylated-Lys
EKKKNLAGGKKR

SEQ ID NO: 269 - protein, 13 aa, Homo sapiens
  MOD_RES (1)..(1): Phosphorylated-Lys
KENQDKKQQKDPR

SEQ ID NO: 270 - protein, 13 aa, Homo sapiens
  MOD_RES (6)..(6): Phosphorylated-Lys
KENQDKKQQKDPR

SEQ ID NO: 271 - protein, 9 aa, Homo sapiens
  MOD_RES (9)..(9): Phosphorylated-Lys
NPKHADGEK

SEQ ID NO: 272 - protein, 15 aa, Homo sapiens
  MOD_RES (13)..(13): Phosphorylated-Lys
MQPQASAIPSPGKFR

SEQ ID NO: 273-281 - skipped (9 entries)

SEQ ID NO: 282 - protein, 32 aa, Homo sapiens
  MOD_RES (31)..(31): Phosphorylated-Lys
EEIQDEEDDDDYVEEGEEEEEEEEGGLRGEKR

SEQ ID NO: 283 - protein, 14 aa, Homo sapiens
  MOD_RES (7)..(7): Phosphorylated-Lys
NLDIDPKLQEYLGK

SEQ ID NO: 284 - protein, 10 aa, Homo sapiens
  MOD_RES (6)..(6): Phosphorylated-Lys
GNSFTKFGNR

SEQ ID NO: 285 - protein, 10 aa, Homo sapiens
  MOD_RES (6)..(6): Phosphorylated-Lys
ELKKQKRAAR

SEQ ID NO: 286 - protein, 10 aa, Homo sapiens
  MOD_RES (5)..(5): Phosphorylated-Lys
IIEDKTFGLK

SEQ ID NO: 287 - protein, 21 aa, Homo sapiens
  MOD_RES (5)..(5): Phosphorylated-Lys
EELTKQVKSHQLVKSLSRVAK

SEQ ID NO: 288 - protein, 21 aa, Homo sapiens
  MOD_RES (21)..(21): Phosphorylated-Lys
EELTKQVKSHQLVKSLSRVAK

SEQ ID NO: 289 - protein, 17 aa, Homo sapiens
  MOD_RES (17)..(17): Phosphorylated-Lys
EEKRKRDSSTQPPKSAK

SEQ ID NO: 290 - protein, 12 aa, Homo sapiens
  MOD_RES (11)..(11): Phosphorylated-Lys
MKAPAKKPLIKR

SEQ ID NO: 291 - protein, 12 aa, Homo sapiens
  MOD_RES (4)..(4): Phosphorylated-Lys
LTMKTDSLMEEK

SEQ ID NO: 292-294 - skipped (3 entries)

SEQ ID NO: 295 - protein, 20 aa, Homo sapiens
  MOD_RES (16)..(16): Phosphorylated-Lys
RPVFPPLCGDGLLSGKEETR

SEQ ID NO: 296 - skipped

SEQ ID NO: 297 - protein, 10 aa, Homo sapiens
  MOD_RES (6)..(6): Phosphorylated-Lys
VTYLGKELLR

SEQ ID NO: 298 - protein, 12 aa, Homo sapiens
  MOD_RES (5)..(5): Phosphorylated-Lys
KVAQKLLNGQRK

SEQ ID NO: 299 - protein, 12 aa, Homo sapiens
  MOD_RES (12)..(12): Phosphorylated-Lys
KVAQKLLNGQRK

SEQ ID NO: 300 - protein, 18 aa, Homo sapiens
  MOD_RES (16)..(16): Phosphorylated-Lys
NQEDNKVKDLNQEIEKLK

SEQ ID NO: 301 - protein, 10 aa, Homo sapiens
  MOD_RES (2)..(2): Phosphorylated-Lys
EKKKVNCKPK

SEQ ID NO: 302 - protein, 25 aa, Homo sapiens
  MOD_RES (22)..(22): Phosphorylated-Lys
SQPNHTPAGPPGPSSNPVSNGKETR

SEQ ID NO: 303 - protein, 10 aa, Homo sapiens
  MOD_RES (3)..(3): Phosphorylated-Lys
LDKVLSDMKR

SEQ ID NO: 304 - protein, 10 aa, Homo sapiens
  MOD_RES (9)..(9): Phosphorylated-Lys
LDKVLSDMKR

SEQ ID NO: 305 - protein, 14 aa, Homo sapiens
  MOD_RES (6)..(6): Phosphorylated-Lys
SPDDLKKKKAKAGK

SEQ ID NO: 306 - protein, 14 aa, Homo sapiens
  MOD_RES (7)..(7): Phosphorylated-Lys
SPDDLKKKKAKAGK

SEQ ID NO: 307 - protein, 14 aa, Homo sapiens
  MOD_RES (9)..(9): Phosphorylated-Lys
SPDDLKKKKAKAGK

SEQ ID NO: 308 - protein, 14 aa, Homo sapiens
  MOD_RES (14)..(14): Phosphorylated-Lys
SPDDLKKKKAKAGK

SEQ ID NO: 309 - protein, 11 aa, Homo sapiens
  MOD_RES (10)..(10): Phosphorylated-Lys
LCFSVQNVFKK

SEQ ID NO: 310 - protein, 18 aa, Homo sapiens
  MOD_RES (10)..(10): Phosphorylated-Lys
FLPTLEQKLKQDQKVLKK

SEQ ID NO: 311 - protein, 12 aa, Homo sapiens
  MOD_RES (5)..(5): Phosphorylated-Lys
FSQAKLLAGAVK

SEQ ID NO: 312 - protein, 11 aa, Homo sapiens
  MOD_RES (5)..(5): Phosphorylated-Lys
EEMIKINKGKR

SEQ ID NO: 313 - protein, 11 aa, Homo sapiens
  MOD_RES (10)..(10): Phosphorylated-Lys
EEMIKINKGKR

SEQ ID NO: 314 - skipped

SEQ ID NO: 315 - protein, 20 aa, Homo sapiens
  MOD_RES (19)..(19): Phosphorylated-Lys
LQQQQRPEDAEDGAEGGGKR

SEQ ID NO: 316 - protein, 11 aa, Homo sapiens
  MOD_RES (7)..(7): Phosphorylated-Lys
VINTGIKVWCR

SEQ ID NO: 317 - protein, 21 aa, Homo sapiens
  MOD_RES (21)..(21): Phosphorylated-Lys
TAGYYPNPPLVLSSDETLISK

SEQ ID NO: 318 - protein, 11 aa, Homo sapiens
  MOD_RES (2)..(2): Phosphorylated-Lys
DKAGKDTARGK

SEQ ID NO: 319 - protein, 11 aa, Homo sapiens
  MOD_RES (5)..(5): Phosphorylated-Lys
DKAGKDTARGK

SEQ ID NO: 320 - protein, 10 aa, Homo sapiens
  MOD_RES (2)..(2): Phosphorylated-Lys
GKHGFSDNQK

SEQ ID NO: 321 - protein, 15 aa, Homo sapiens
  MOD_RES (12)..(12): Phosphorylated-Lys
SAEEEAADLPTKPTK

SEQ ID NO: 322 - protein, 14 aa, Homo sapiens
  MOD_RES (3)..(3): Phosphorylated-Lys
ISKFGFAIGSQTTK

SEQ ID NO: 323-325 - skipped (3 entries)

SEQ ID NO: 326 - protein, 14 aa, Homo sapiens
  MOD_RES (3)..(3): Phosphorylated-Lys
CSKLPSSTKSGWPR

SEQ ID NO: 327 - protein, 14 aa, Homo sapiens
  MOD_RES (9)..(9): Phosphorylated-Lys
CSKLPSSTKSGWPR

SEQ ID NO: 328-330 - skipped (3 entries)

SEQ ID NO: 331 - protein, 11 aa, Homo sapiens
  MOD_RES (9)..(9): Phosphorylated-Lys
DTDMNKKDKQK

SEQ ID NO: 332 - protein, 17 aa, Homo sapiens
  MOD_RES (16)..(16): Phosphorylated-Lys
LFVGNLPTDITEEDFKR

SEQ ID NO: 333 - protein, 10 aa, Homo sapiens
  MOD_RES (10)..(10): Phosphorylated-Lys
GGPDFSNLEK

SEQ ID NO: 334 - skipped

SEQ ID NO: 335 - protein, 8 aa, Homo sapiens
  MOD_RES (2)..(2): Phosphorylated-Lys
NKKRRKKR

SEQ ID NO: 336 - protein, 8 aa, Homo sapiens
  MOD_RES (3)..(3): Phosphorylated-Lys
NKKRRKKR

SEQ ID NO: 337 - protein, 8 aa, Homo sapiens
  MOD_RES (7)..(7): Phosphorylated-Lys
NKKRRKKR

SEQ ID NO: 338 - protein, 12 aa, Homo sapiens
  MOD_RES (6)..(6): Phosphorylated-Lys
QREARKRALARK

SEQ ID NO: 339 - protein, 12 aa, Homo sapiens
  MOD_RES (12)..(12): Phosphorylated-Lys
QREARKRALARK

SEQ ID NO: 340-342 - skipped (3 entries)

SEQ ID NO: 343 - protein, 15 aa, Homo sapiens
  MOD_RES (14)..(14): Phosphorylated-Lys
DCHHLGIQNNFDYKR

SEQ ID NO: 344 - protein, 13 aa, Homo sapiens
  MOD_RES (4)..(4): Phosphorylated-Lys
SIFKPFIFVDDVK

SEQ ID NO: 345 - protein, 18 aa, Homo sapiens
  MOD_RES (2)..(2): Phosphorylated-Lys
HKKSLQKLVSDWNTLKSR

SEQ ID NO: 346 - protein, 18 aa, Homo sapiens
  MOD_RES (3)..(3): Phosphorylated-Lys
HKKSLQKLVSDWNTLKSR

SEQ ID NO: 347 - protein, 18 aa, Homo sapiens
  MOD_RES (7)..(7): Phosphorylated-Lys
HKKSLQKLVSDWNTLKSR

SEQ ID NO: 348 - protein, 13 aa, Homo sapiens
  MOD_RES (7)..(7): Phosphorylated-Lys
QSVQPSKFNSYGR

SEQ ID NO: 349-351 - skipped (3 entries)

SEQ ID NO: 352 - protein, 11 aa, Homo sapiens
  MOD_RES (10)..(10): Phosphorylated-Lys
TYGCVPVANKR

SEQ ID NO: 353 - protein, 11 aa, Homo sapiens
  MOD_RES (6)..(6): Phosphorylated-Lys
TLILGKAVNAK

SEQ ID NO: 354-355 - skipped (2 entries)

SEQ ID NO: 356 - protein, 11 aa, Homo sapiens
  MOD_RES (6)..(6): Phosphorylated-Lys
NSATFKSFEDR

SEQ ID NO: 357 - protein, 15 aa, Homo sapiens
  MOD_RES (3)..(3): Phosphorylated-Lys
SEKESSRGRGNNNRK

SEQ ID NO: 358-359 - skipped (2 entries)

SEQ ID NO: 360 - protein, 13 aa, Homo sapiens
  MOD_RES (5)..(5): Phosphorylated-Lys
LEKGKFGMVLLKK

SEQ ID NO: 361 - protein, 13 aa, Homo sapiens
  MOD_RES (11)..(11): Phosphorylated-Lys
LLEAISSLDGKNR

SEQ ID NO: 362 - skipped

SEQ ID NO: 363 - protein, 13 aa, Homo sapiens
  MOD_RES (6)..(6): Phosphorylated-Lys
ISSIGKFISGTPR

SEQ ID NO: 364 - protein, 19 aa, Homo sapiens
  MOD_RES (18)..(18): Phosphorylated-Lys
RPDFAQQQAMQQLTFDGKR

SEQ ID NO: 365 - protein, 12 aa, Homo sapiens
  MOD_RES (1)..(1): Phosphorylated-Lys
KNELPDGLNKKR

SEQ ID NO: 366 - protein, 12 aa, Homo sapiens
  MOD_RES (11)..(11): Phosphorylated-Lys
KNELPDGLNKKR

SEQ ID NO: 367 - protein, 16 aa, Homo sapiens
  MOD_RES (11)..(11): Phosphorylated-Lys
SEQPTLVTASKDGYFK

SEQ ID NO: 368 - protein, 19 aa, Homo sapiens
  MOD_RES (7)..(7): Phosphorylated-Lys
MEASVPKYKKHLPQLGMQK

SEQ ID NO: 369 - protein, 19 aa, Homo sapiens
  MOD_RES (9)..(9): Phosphorylated-Lys
MEASVPKYKKHLPQLGMQK

SEQ ID NO: 370 - protein, 13 aa, Homo sapiens
  MOD_RES (10)..(10): Phosphorylated-Lys
KFPTAIFESKGFR

SEQ ID NO: 371 - skipped

SEQ ID NO: 372 - protein, 24 aa, Homo sapiens
  MOD_RES (23)..(23): Phosphorylated-Lys
VLSLDDYFITEVEKEEKDPDSGKK

SEQ ID NO: 373-376 - skipped (4 entries)

SEQ ID NO: 377 - protein, 17 aa, Homo sapiens
  MOD_RES (6)..(6): Phosphorylated-Lys
QLSMEKFLSLTEEDLNK

SEQ ID NO: 378 - protein, 20 aa, Homo sapiens
  MOD_RES (19)..(19): Phosphorylated-Lys
YQVSSLCGTDNEDKITTGKR

SEQ ID NO: 379 - protein, 9 aa, Homo sapiens
  MOD_RES (3)..(3): Phosphorylated-Lys
KKKKSKILK

SEQ ID NO: 380 - protein, 9 aa, Homo sapiens
  MOD_RES (4)..(4): Phosphorylated-Lys
KKKKSKILK

SEQ ID NO: 381 - protein, 17 aa, Homo sapiens
  MOD_RES (14)..(14): Phosphorylated-Lys
LGAVPATSGPTTFKQQR

SEQ ID NO: 382 - protein, 8 aa, Homo sapiens
  MOD_RES (1)..(1): Phosphorylated-Lys
KVTAAEKK

SEQ ID NO: 383 - protein, 8 aa, Homo sapiens
  MOD_RES (7)..(7): Phosphorylated-Lys
KVTAAEKK

SEQ ID NO: 384-385 - skipped (2 entries)

SEQ ID NO: 386 - protein, 27 aa, Homo sapiens
  MOD_RES (15)..(15): Phosphorylated-Lys
LFTAESLIGLKNPEKSFPVNSDVGVLK

SEQ ID NO: 387 - protein, 17 aa, Homo sapiens
  MOD_RES (14)..(14): Phosphorylated-Lys
CAIGVTHFQLVQMKDFR

SEQ ID NO: 388 - protein, 12 aa, Homo sapiens
  MOD_RES (4)..(4): Phosphorylated-Lys
GAFKAFTGEGQK

SEQ ID NO: 389 - protein, 17 aa, Homo sapiens
  MOD_RES (16)..(16): Phosphorylated-Lys
VKVGKEDSSSAEFLEKR

SEQ ID NO: 390 - protein, 11 aa, Homo sapiens
  MOD_RES (5)..(5): Phosphorylated-Lys
LQTEKQELLQK

SEQ ID NO: 391 - protein, 7 aa, Homo sapiens
  MOD_RES (1)..(1): Phosphorylated-Lys
KAIQDMF